SEQUENCE LISTING (2 sequences)

SEQ ID NO: 1 - DNA, 21 bp, Homo sapiens
  source 1..21: mol_type = genomic DNA
catcagccctaatccatctga

SEQ ID NO: 2 - DNA, 21 bp, Homo sapiens
  source 1..21: mol_type = genomic DNA
cgcgactaacaatcaaagtga

(19) United States
(12) Patent Application Publication
(10) Pub. No.: US 2025/0034127 A1
(43) Pub. Date: Jan. 30, 2025

(54) AUTOPHAGY INDUCING COMPOUNDS AND USES THEREOF, IN PARTICULAR FOR DISEASES OF THE CNS

(71) Applicants: Samsara Therapeutics Inc., Lewes, DE (US); Institut National de la Santé et de la Recherche Médicale (INSERM), Paris (FR); Université Paris Cité, Paris (FR); Sorbonne Université, Paris (FR); Assistance Publique-Hôpitaux De Paris (APHP), Paris (FR)

(72) Inventors: Peter Hamley, Oxford (GB); Warren Galloway, Kronberg im Taunus (DE); Oliver Kepp, Paris (FR); Guido Kroemer, Paris (FR)

(21) Appl. No.: 18/711,519

(22) PCT Filed: Nov. 17, 2022
(86) PCT No.: PCT/EP2022/082315

§ 371 Date: May 17, 2024

(30) Foreign Application Priority Data
Nov. 17, 2021 (EP) 21208821.5

(51) Int. Cl. C07D 417/14; A61K 31/427; A61K 31/454; A61K 31/496; A61P 25/16; A61P 25/28

(52) U.S. Cl. CPC C07D 417/14; A61K 31/427; A61K 31/454; A61K 31/496; A61P 25/16; A61P 25/28

(57) ABSTRACT

The invention relates to pharmaceutical compositions and methods of treating autophagy related diseases and disorders, and in particular neurological and/or CNS-related diseases. The present invention relates to compounds according to Formula (I) or salts, solvates and/or hydrates thereof, wherein said compounds induce and/or stimulate the process of autophagy, as well as uses of the compounds in the treatment and prevention of autophagy related diseases and disorders. Examples are cancer, age-related diseases, and viral infection, in particular of the CNS.

The invention relates to pharmaceutical compositions and methods of treating autophagy related diseases and disorders, and in particular neurological and/or CNS-related diseases. The present invention relates to compounds according to Formula (I) or salts, solvates and/or hydrates thereof, wherein said compounds induce and/or stimulate the process of autophagy, as well as uses of the compounds in the treatment and prevention of autophagy related diseases and disorders. Examples are cancer, age-related diseases, and viral infection, in particular of the CNS.

### BACKGROUND OF THE INVENTION

Autophagy is the process of removing unnecessary organelles and proteins from cells that are lost or lost function, it helps maintain cell homeostasis and is a cell survival mechanism.

The cell-autonomous antimicrobial defense functions of autophagy, demonstrated initially in the case of streptococci and Mycobacterium tuberculosis have been extended to a wide variety of microbes with a caveat that most highly adapted pathogens have evolved specific protective mechanisms against autophagic elimination of microbes. Other studies have uncovered orderly intersections between autophagy and innate and adaptive immunity, T cell development, differentiation and homeostasis, and inflammatory responses. Thus, autophagy plays a large role in various diseases such as cancer, inflammatory disease, degenerative neurological disease, and immune disease.

Autophagy is a cell survival mechanism that is induced in stressed cells

Towers and Thorburn (in: Therapeutic Targeting of Autophagy. EBioMedicine. 2016; 14:15-23. doi: 10.1016/j.ebiom.2016.10.034) disclose that autophagy is widely accepted as cytoprotective against neurodegenerative diseases and a variety of clinical interventions are moving forward to increase autophagy as a therapeutic intervention. Autophagy has both positive and negative roles in cancer and this has led to controversy over whether or how autophagy manipulation should be attempted in cancer therapy. Nevertheless, cancer is the disease where most current activity in trying to manipulate autophagy for therapy is taking place and dozens of clinical trials are using autophagy inhibition with Chloroquine or Hydroxychloroquine in combination with other drugs for the treatment of multiple neoplasms. They review recent literature implicating autophagy in neurodegenerative diseases and cancer and highlight some of the opportunities, controversies and potential pitfalls of therapeutically targeting autophagy.

Mulcahy Levy and Thorburn (in: Autophagy in cancer: moving from understanding mechanism to improving therapy responses in patients. Cell Death Differ 27, 843-857 (2020). https://doi.org/10.1038/s41418-019-0474-7) disclose that autophagy allows for cellular material to be delivered to lysosomes for degradation resulting in basal or stress-induced turnover of cell components that provide energy and macromolecular precursors. These activities are thought to be particularly important in cancer where both tumor-promoting and tumor-inhibiting functions of autophagy have been described. Autophagy has also been intricately linked to apoptosis and programmed cell death, and understanding these interactions is becoming increasingly important in improving cancer therapy and patient outcomes. In the review, they consider how recent discoveries about how autophagy manipulation elicits its effects on cancer cell behavior can be leveraged to improve therapeutic responses.

Grainger et al. (1995, Nature Medicine 1:1067-1073) and Reckless et al. (1997, Circulation 95:1542-1548) have demonstrated that tamoxifen, a potent inducer of autophagy, inhibited atherosclerosis in mice models by suppressing the diet-induced formation of lipid lesions in the aorta by lowering of low-density lipoprotein (LDL) cholesterol.

CN 102516239A discloses aromatic thiazole micro-molecular organic compounds with the structural formula represented by Formula (I), or hydrates thereof or pharmaceutically acceptable salts thereof. The compounds of the invention or compositions containing the compounds can be used for sun protection, anti-ultraviolet and skin damage protection as a cosmetic additive and for inhibition of the cell apoptosis caused by excess ultraviolet irradiation and the expression of cyclooxygenase COX2.

US 2004-0116425A1 discloses related compounds useful in the treatment of diseases associated with prenylation of proteins and pharmaceutically acceptable salts thereof, to pharmaceutical compositions comprising same, and to methods for inhibiting protein prenylation in an organism using the same.

WO 2009-103432A2 extremely broadly relates to molecular probes of the formula (I) L1-R1-L-A-X as defined herein that allow for the observation of the catalytic activity of a selected caspase, cathepsin, MMP and carboxypeptidase in in vitro assays, in cells or in multicellular organisms.

In WO 2010-147653A1 compounds are extremely broadly disclosed for treating ophthalmic conditions related to mislocalization of opsin proteins, the misfolding of mutant opsin proteins and the production of toxic visual cycle products that accumulate in the eye.

WO 2017-216579A1 relates to a heterocyclic compound 1,1′-(((propane-2,2-diylbis(4,1-phenylene))bis(oxy))bis(ethane-2,1-diyl))dipyrrolidine and its medical uses, for example as an autophagy inducer.

The development of more selective autophagy inducers is needed if they are to become medicinally useful in the treatment and/or prevention of diseases where autophagy plays a role, in particular in the central nervous system (CNS). It is an object of the present invention to provide effective agents that can be used for the prevention and treatment of conditions and diseases that can be treated/prevented by inducing and/or stimulating of autophagy, in particular cancer, age-related diseases, and infections. Other objects and advantages will become apparent to the person of skill when studying the present description of the present invention.

In a first aspect of the present invention, the above object is solved by a compound according to Formula (I),

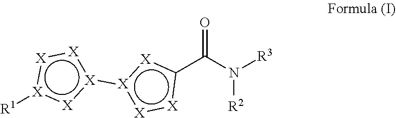

wherein
X is independently selected from chemically possible combinations of C, N, O, and S, and is optionally substituted with —CH₃, —CH₂—CH₃ or COOH,
R¹ is selected from cyclic C₅ or C₆ alkyl, optionally including chemically possible N, O, and/or S, and optionally mono- or bi-substituted with —CH₃, —NH₂, —COOH, C₁ to C₄ alkoxy, halo, trifluoromethyl, trifluoromethoxy;
R² is selected from H, CH₃, straight or branched C₂ to C₆ alkyl, optionally including chemically possible N, O, and/or S, and optionally substituted with —CH₃, —NH₂, —OH, —COOH,
cyclopentyl, cyclohexyl, bicyclo[2.2.1]heptane, bicyclo[3.1.1]heptane, bicyclo[2.2.2]octane, optionally including chemically possible N, O, and/or S, and optionally substituted with —CH₃, —CH₂—CH₃, —OH, -isopropyl, —COOH, —COOCH₃, —CH₂-cyclohexyl, —NH₂,
R³ is selected from H, straight or branched C₁ to C₆ alkyl, optionally substituted with —CH₃, —OH, —CH₂—CH₃, —NH₂, —CH₂—NH₂, —CH₂—NH—CH₃, —COOH, optionally including chemically possible one or more N, O, and/or S;
C₁ to C₄ alkoxy, optionally including chemically possible one or more N, O, and/or S, and optionally mono- or bi substituted with, —NH₂, —OH, —COOH;
cyclopropyl, cyclobutyl, optionally including chemically possible one or more N, O, and/or S, and optionally mono- or bi substituted with —CH₃, —CH₂—CH₃, —NH₂, —CH₂—NH₂, —CH₂—NH—CH₃, —OH, —COOH;
with the provisio that R² and R³ are not both H or CH₃,
R² and R³ may be connected to form a 4-membered, 5-membered, 6-membered, 7-membered ring, a bicyclic ring structure of fused cyclopentyl and/or cyclohexyl, bicyclo[3.1.1]heptane, optionally including chemically possible one or more N, O, and/or S, and optionally being mono- or bi-substituted with straight or branched C₁ to C₆ alkyl, optionally substituted with —CH₃, —OH, —CH₂—CH₃, —NH₂, —NH—CH₃, —CH₂—NH₂, —CH₂—NH—CH₃, —OH, —COOH, —COOCH₃, ═O, isopropyl, sulfonamide, and optionally including chemically possible one or more N, O, and/or S;
with the provisio that either R² or R³ or the combination thereof include at least one saturated/fully substituted nitrogen,
a physiologically acceptable salt, a solvate, a hydrate, an enantiomer or a polymorph thereof.

The compounds described herein were shown surprisingly to be highly effective autophagy inducers/stimulators. The present inventors thus invented compounds for the induction and/or stimulation of autophagy in disease—and undesired conditions-related cells of a patient or subject. In the case of a compound represented by the general Formula according to the present invention, the autophagy is effectively induced and/or stimulated. It was surprisingly found that the compounds according to Formula I, as an autophagy inducer, were effective and exhibit many advantages compared to other autophagy-related treatment options. In comparison with prior art compounds as tested (including data not shown), an improvement of the present invention lies in the unexpected observation that the compounds described herein are highly effective autophagy inducers (see Examples below). Furthermore, the compounds exhibit higher concentrations inside compared to outside of the CNS or brain (peripheral), preferably a minimum of a 2:1 brain/CNS to peripheral ratio, after an administration thereof, which makes them ideal candidates for the induction and/or stimulation of autophagy in disease—and undesired conditions-related cells of a patient or subject involving the CNS and brain.

Based on these results this is evidence that these compounds are active in autophagy-related diseases as disclosed herein. Furthermore, examples for this activity are shown in the Examples below which also relate to specific activities in models of viral infections and diseases involving protein misfolding. By way of an exemplar medical application, the compounds according to Formula 157

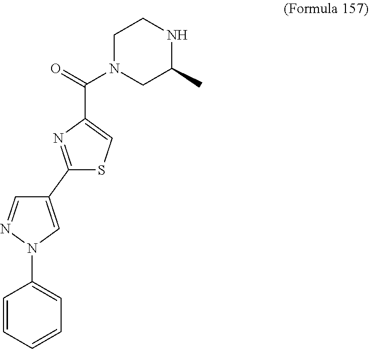 [SMILES: C[C@H]1CN(C(=O)c2csc(-c3cnn(-c4ccccc4)c3)n2)CCN1]

as well as compounds 185 and 196 were surprisingly efficacious (see Examples below).

Preferred is a compound according to the present invention according to the following formula II,

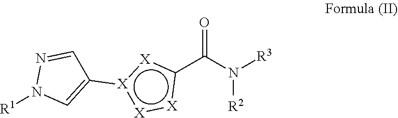

wherein X, R¹, R² and R³ are as above, a physiologically acceptable salt, a solvate, a hydrate, an enantiomer or a polymorph thereof.

Preferred is a compound according to the present invention according to the following formula II,

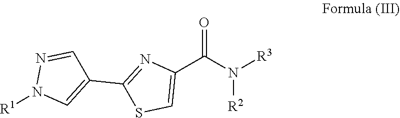

wherein R¹, R² and R³ are as above, a physiologically acceptable salt, a solvate, a hydrate, an enantiomer or a polymorph thereof.

Preferred is a compound according to the present invention, wherein R¹ is selected from

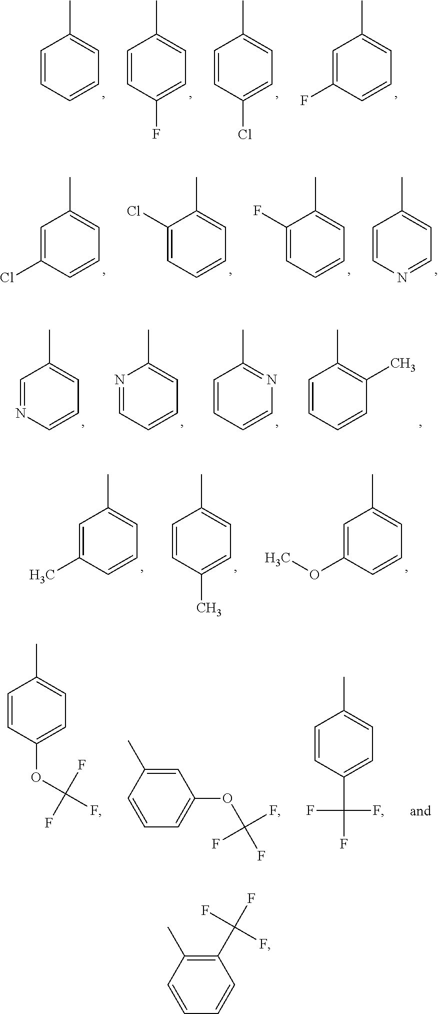

a physiologically acceptable salt, a solvate, a hydrate, an enantiomer or a polymorph thereof.

Preferred is a compound according to the present invention, wherein R′ and X are as above. R² is selected from H, —CH₃ . . . cyclopentyl, cyclohexyl,

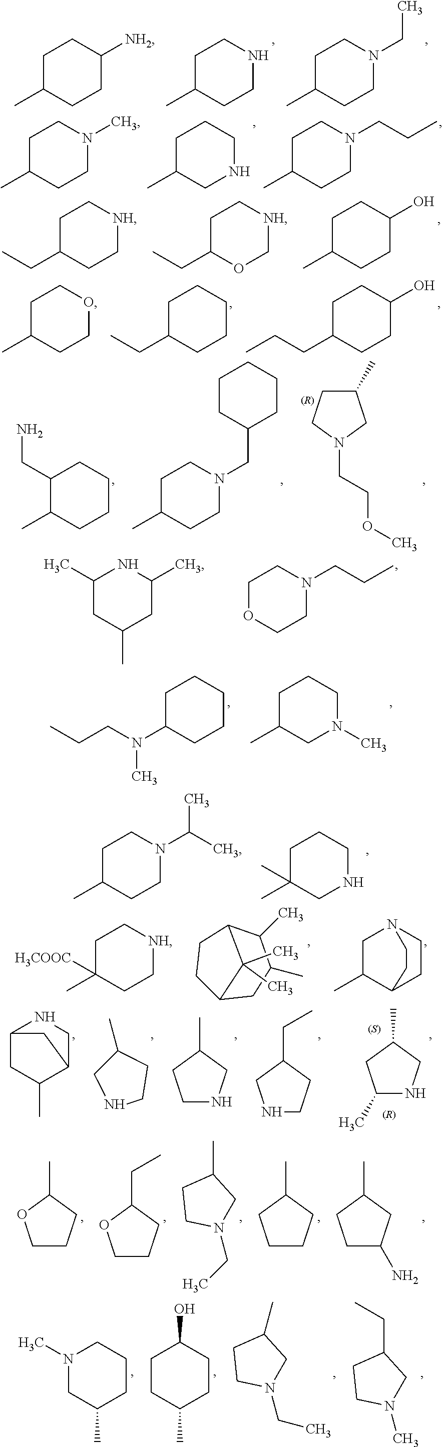

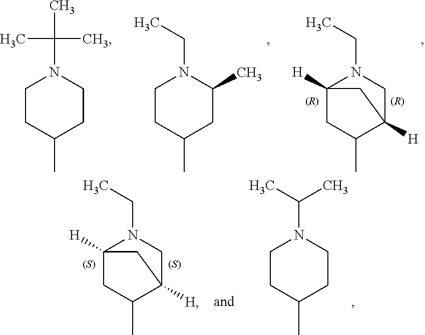

R³ is selected from H, —CH₃, —CH—CH₃, —CH—CH—CH₃; propyl, isopropyl, —CH—CH₂—OH, —CH—CH₂—NH₂, —CH₂—CH—CH₂—NH₂, —CH—CH—CH—CH—CH₂—NH₂, —CH—CH₂—O—CH—CH₂—NH₂, —CH—CH(CH₃)₂, —CH—CH(NH₂)—CH₃, —CH(—CH₃)—CH₂—NH₂, —CH—CH₂—N(CH₃)₂,

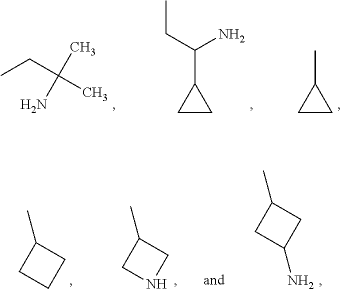

and R² and R³ may be connected to form a ring selected from

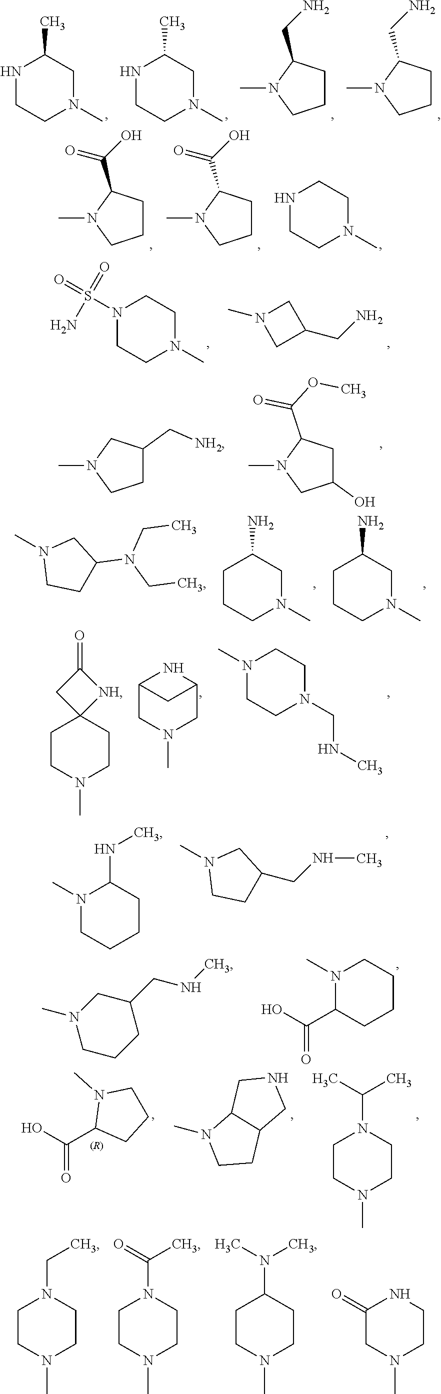

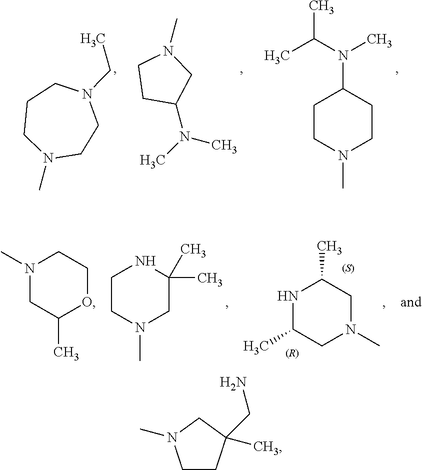

a physiologically acceptable salt, a solvate, a hydrate, an enantiomer or a polymorph thereof.

Preferred is a compound according to the present invention according to the following formula IV,

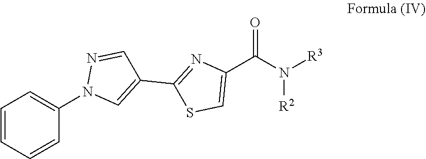

wherein R² is selected from

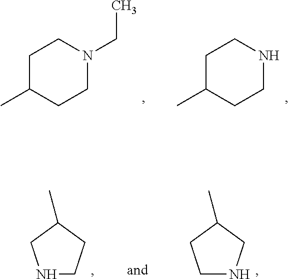

R³ is selected from H, CH₃, —CH₂—CH₂—CH₃, and isopropyl, or
R² and R³ form a ring selected from

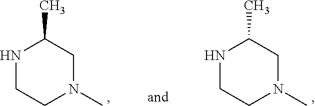

a physiologically acceptable salt, a solvate, a hydrate, an enantiomer or a polymorph thereof.

Preferred is a compound according to the present invention, wherein R² and R³ or the combination thereof together include at least two nitrogens selected from the group of saturated nitrogens and at least partially unsaturated nitrogens that are protected by at least one chemical group in an ortho-position thereof.

Preferred is a compound according to the present invention selected from the Formulae 1 to 304, in particular
N-(1-ethylpiperidin-4-yl)-N-methyl-5-(1-phenyl-1H-pyrazol-4-yl)-1H-pyrrole-2-carboxamide (Formula 1);
(3S)-3-methyl-1-[4-(5-phenyl-1,3,4-thiadiazol-2-yl)thiophene-2-carbonyl]piperazine (Formula 2);
(3S)-3-methyl-1-[4-(1-phenyl-1H-pyrazol-4-yl)-1H-pyrrole-2-carbonyl]piperazine (Formula 3);
(3S)-3-methyl-1-[5-(1-phenyl-1H-pyrazol-4-yl)thiophene-3-carbonyl]piperazine (Formula 4);
(3S)-3-methyl-1-[2-(5-phenylthiophen-2-yl)-1,3-thiazole-4-carbonyl]piperazine (Formula 7);
(3S)-3-methyl-1-[2-(2-phenyl-1,3-oxazol-5-yl)-1,3-thiazole-4-carbonyl]piperazine (Formula 13);
(3S)-3-methyl-1-(2-{1-[3-(trifluoromethoxy)phenyl]-1H-pyrazol-4-yl}-1,3-thiazole-4-carbonyl) piperazine (Formula 24);
(3S)-3-methyl-1-{2-[1-(3-methylphenyl)-1H-pyrazol-4-yl]-1,3-thiazole-4-carbonyl}piperazine (Formula 27);
(3S)-1-{2-[1-(3-chlorophenyl)-1H-pyrazol-4-yl]-1,3-thiazole-4-carbonyl}-3-methylpiperazine (Formula 30);
(3S)-3-methyl-1-{2-[1-(pyridin-2-yl)-1H-pyrazol-4-yl]-1,3-thiazole-4-carbonyl}piperazine (Formula 33);
(3S)-1-{2-[1-(4-fluorophenyl)-1H-pyrazol-4 yl]-1,3-thiazole-4-carbonyl}-3-methylpiperazine (Formula 34);
(3S)-3-methyl-1-(2-{1-[4-(trifluoromethoxy)phenyl]-1H-pyrazol-4-yl}-1,3-thiazole-4-carbonyl) piperazine (Formula 37);
(3S)-3-methyl-1-(2-{1-[3-(trifluoromethyl)phenyl]-1H-pyrazol-4-yl}-1,3-thiazole-4-carbonyl) piperazine (Formula 38);
(3S)-1-{2-[1-(4-chlorophenyl)-1H-pyrazol-4-yl]-1,3-thiazole-4-carbonyl}-3-methylpiperazine (Formula 39);
(3S)-1-{2-[1-(3-methoxyphenyl)-1H-pyrazol-4-yl]-1,3-thiazole-4 carbonyl}-3-methylpiperazine (Formula 62);
(3S)-3-methyl-1-{2-[1-(4-methylphenyl)-1H-pyrazol-4-yl]-1,3-thiazole-4-carbonyl}piperazine (Formula 63);
(3S)-3-methyl-1-[5-(1-phenyl-1H-pyrazol-4-yl)-1H-pyrrole-2-carbonyl]piperazine (Formula 67);
(3S)-3-methyl-1-(2-{1-[2-(trifluoromethyl)phenyl]-1H-pyrazol-4-yl}-1,3-thiazole-4-carbonyl) piperazine (Formula 72);
(3S)-3-methyl-1-(2-{1-[4-(trifluoromethyl)phenyl]-1H-pyrazol-4-yl}-1,3-thiazole-4-carbonyl) piperazine (Formula 75);
(3S)-1-{2-[1-(2-chlorophenyl)-1H-pyrazol-4-yl]-1,3-thiazole-4-carbonyl}-3-methylpiperazine (Formula 79);
(3S)-3-methyl-1-{2-[1-(2-methylphenyl)-1H-pyrazol-4-yl]-1,3-thiazole-4-carbonyl}piperazine (Formula 85);
(3S)-1-{2-[1-(2-fluorophenyl)-1H-pyrazol-4-yl]-1,3-thiazole-4-carbonyl}-3-methylpiperazine (Formula 96);
(3S)-3-methyl-1-[5-(1-phenyl-1H-pyrazol-4-yl) furan-2-carbonyl]piperazine (Formula 102);
N-(1-ethylpiperidin-4-yl)-4-(1-phenyl-1H-pyrazol-4-yl)thiophene-2-carboxamide (Formula 103);
N-(1-ethylpiperidin-4-yl)-5-(1-phenyl-1H-pyrazol-4-yl) furan-2-carboxamide (Formula 109);
(3S)-3-methyl-1-[4-(1-phenyl-1H-pyrazol-4-yl)thiophene-2-carbonyl]piperazine (Formula 113);
(3R)-3-methyl-1-[2-(1-phenyl-1H-pyrazol-4-yl)-1,3-thiazole-4-carbonyl]piperazine (Formula 118);
N,N-diethyl-1-[2-(1-phenyl-1H-pyrazol-4-yl)-1,3-thiazole-4-carbonyl]pyrrolidin-3-amine (Formula 134);
N-(1-methylpiperidin-4-yl)-2-(1-phenyl-1H-pyrazol-4-yl)-1,3-thiazole-4-carboxamide (Formula 138);
(3S)-3-methyl-1-[2-(1-phenyl-1H-pyrazol-4-yl)-1,3-thiazole-4-carbonyl]piperazine (Formula 157);
N-(1-ethylpiperidin-4-yl)-2-(1-phenyl-1H-pyrazol-4-yl)-1,3-thiazole-4-carboxamide (Formula 185);
1-ethyl-4-[2-(1-phenyl-1H-pyrazol-4-yl)-1,3-thiazole-4 carbonyl]-1,4-diazepane (Formula 193);
N-methyl-2-(1-phenyl-1H-pyrazol-4-yl)-N-[1-(propan-2-yl) piperidin-4-yl]-1,3-thiazole-4-carboxamide (Formula 195);
N-(1-ethylpiperidin-4-yl)-N-methyl-2-(1-phenyl-1H-pyrazol-4-yl)-1,3-thiazole-4 carboxamide (Formula 196);
N,N-dimethyl-1-[2-(1-phenyl-1H-pyrazol-4-yl)-1,3-thiazole-4-carbonyl]piperidin-4-amine (Formula 197);
N-[(3S)-1-methylpiperidin-3-yl]-2-(1-phenyl-1H-pyrazol-4-yl)-1,3-thiazole-4 carboxamide (Formula 208);
1-[2-(1-phenyl-1H-pyrazol-4-yl)-1,3-thiazole-4-carbonyl]-4-(propan-2-yl) piperazine (Formula 209);
   a physiologically acceptable salt, a solvate, a hydrate, an enantiomer or a polymorph thereof.

Further preferred is a compound according to the present invention selected from the following:
N-(1-ethylpiperidin-4-yl)-N-methyl-5-(1-phenyl-1H-pyrazol-4-yl)-1H-pyrrole-2-carboxamide (Formula 1);
(3S)-3-methyl-1-[2-(1-phenyl-1H-pyrazol-4-yl)-1,3-thiazole-4-carbonyl]piperazine (Formula 157);
N-(1-ethylpiperidin-4-yl)-2-(1-phenyl-1H-pyrazol-4-yl)-1,3-thiazole-4-carboxamide (Formula 185);
N-(1-ethylpiperidin-4-yl)-N-methyl-2-(1-phenyl-1H-pyrazol-4-yl)-1,3-thiazole-4 carboxamide (Formula 196);
N-(1-ethylpiperidin-4-yl)-N-methyl-2-[1-(pyridin-3-yl)-1H-pyrazol-4-yl]-1,3-thiazole-4-carboxamide (Formula 224);
2-(4-{4-[(3S)-3-methylpiperazine-1-carbonyl]-1,3-thiazol-2-yl}-1H-pyrazol-1-yl) pyrazine (Formula 232);
N-(1-ethylpiperidin-4-yl)-N-methyl-2-[1-(pyrazin-2-yl)-1H-pyrazol-4-yl]-1,3-thiazole-4-carboxamide (Formula 233);
   a physiologically acceptable salt, a solvate, a hydrate, an enantiomer or a polymorph thereof.

The term “pharmaceutically acceptable salt” refers to a pharmaceutically acceptable organic or inorganic salt of the compound of the invention. This may include addition salts of inorganic acids such as hydrochloride, hydrobromide, hydroiodide, sulphate, phosphate, diphosphate and nitrate or of organic acids such as acetate, maleate, fumarate, tartrate, succinate, citrate, lactate, methanesulphonate, p-toluenesulphonate, palmoate and stearate. Exemplary salts also include oxalate, chloride, bromide, iodide, bisulphate, acid phosphate, isonicotinate, salicylate, acid citrate, oleate, tannate, pantothenate, bitartrate, ascorbate, gentisinate, gluconate, glucuronate, saccharate, formate, benzoate, glutamate, ethanesulfonate, and benzenesulfonate salts. For other examples of pharmaceutically acceptable salts, reference can be made to Gould (1986, Int J Pharm 33:201-217).

According to a further aspect of the invention, there is a provided a method for producing a compound according to the present invention, comprising the steps according to any one of general experimental procedures, in particular 1 to 5, as disclosed herein and below.

According to a further aspect of the invention, there is a provided a pharmaceutical composition comprising a pharmaceutically effective amount of the compound according to the present invention, and a pharmaceutically or therapeutically acceptable excipient or carrier.

The term “pharmaceutically or therapeutically acceptable excipient or carrier” refers to a solid or liquid filler, diluent or encapsulating substance which does not interfere with the effectiveness or the biological activity of the active ingredients and which is not toxic to the host, which may be either humans or animals, to which it is administered. Depending upon the particular route of administration, a variety of pharmaceutically-acceptable carriers such as those well known in the art may be used. Non-limiting examples include sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water. Pharmaceutically acceptable carriers or excipients also include diluents (fillers, bulking agents, e.g. lactose, microcrystalline cellulose), disintegrants (e.g. sodium starch glycolate, croscarmellose sodium), binders (e.g. PVP, HPMC), lubricants (e.g. magnesium stearate), glidants (e.g. colloidal SiO₂), solvents/co-solvents (e.g. aqueous vehicle, Propylene glycol, glycerol), buffering agents (e.g. citrate, gluconates, lactates), preservatives (e.g. Na benzoate, parabens (Me, Pr and Bu), BKC), anti-oxidants (e.g. BHT, BHA, Ascorbic acid), wetting agents (e.g. polysorbates, sorbitan esters), thickening agents (e.g. methylcellulose or hydroxyethylcellulose), sweetening agents (e.g. sorbitol, saccharin, aspartame, acesulfame), flavoring agents (e.g. peppermint, lemon oils, butterscotch, etc.), humectants (e.g. propylene, glycol, glycerol, sorbitol). Other suitable pharmaceutically acceptable excipients are inter alia described in Remington's Pharmaceutical Sciences, 15^{th} Ed., Mack Publishing Co., New Jersey (1991) and Bauer et al., Pharmazeutische Technologic, 5^{th} Ed., Govi-Verlag Frankfurt (1997). The person skilled in the art knows suitable formulations for the compounds according to the present invention, and will readily be able to choose suitable pharmaceutically acceptable carriers or excipients, depending, e.g., on the formulation and administration route of the pharmaceutical composition.

All suitable modes of administration are contemplated according to the invention. For example, administration of the medicament may be via oral, subcutaneous, direct intravenous, slow intravenous infusion, continuous intravenous infusion, intravenous or epidural patient controlled analgesia (PCA and PCEA), intramuscular, intrathecal, epidural, intracistemal, intraperitoneal, transdermal, topical, buccal, sublingual, transmucosal, inhalation, intra-atricular, intranasal, rectal or ocular routes, abuse deterrent and abuse resistant formulations, sterile solutions suspensions and depots for parenteral use, and the like, administered as immediate release, sustained release, delayed release, controlled release, extended release and the like. The medicament may be formulated in discrete dosage units and can be prepared by any of the methods well known in the art of pharmacy. The pharmaceutical composition of the present invention can be formulated using methods known in the art to provide rapid, sustained or delayed release of the active ingredient after administration to a mammal. In general, at least one compound according to the present invention is admixed with at least one pharmaceutically acceptable carrier and/or excipient.

In addition to the aforementioned compounds of the invention, the pharmaceutical composition can contain two or more compounds according to the present invention and also other therapeutically active substances.

The dosage of the pharmaceutical composition according to the present invention can be appropriately selected according to the route of administration, the subject to be administered, the target disease and its severity, age, sex weight, individual differences and disease state. Dosage may be repeated several times a day.

According to the present invention, a mammalian subject can be preferably selected from a mouse, rat, cat, dog, rabbit, goat, sheep, horse, camel, lama, cow, monkey, a farm animal, a sport animal, and a pet, and a human.

Further provided is a compound of the invention as defined herein for use in the prevention and/or treatment of conditions and/or diseases in a mammalian subject, such as a human. A condition and/or disease suitable for treatment according to the relevant aspects of the invention is one which is characterized by defective or insufficient autophagy or which would benefit from modulation such as induction of autophagy.

The invention further encompasses the use of a compound of the invention as an autophagy inducer. The use may be a cosmetic use and/or in vitro, for example in an in vitro assay.

U.S. Pat. No. 9,138,400, for example, relates to a cosmetic process for detoxifying the skin and/or for combating cutaneous aging, comprising the topical application on the skin of a composition that comprises at least one activator of the autophagy of cells of the skin. Eckhart L, Tschachler E, and Gruber F (in: Autophagic Control of Skin Aging. Front Cell Dev Biol. 2019; 7:143. Published 2019 Jul. 30. doi: 10.3389/fcell.2019.00143) review the evidence for cell type-specific roles of autophagy in the skin and their differential contributions to aging.

Modified or altered autophagy has been shown to be relevant in neurodegenerative disease, as demonstrated by the accumulation of protein aggregates, for example in Alzheimer disease, Parkinson's disease, polyglutamine diseases, muscle diseases, and amyotrophic lateral sclerosis. Modified autophagy has also been implicated in other neurological diseases including epilepsies, neurometabolic and neurodevelopmental disorders such as schizophrenia.

A crucial role for therapy-induced autophagy in cancer cells has recently emerged, in modulating the interface of cancer cells and the immune system; primarily, by affecting the nature of danger signaling (i.e., the signaling cascade that facilitates the exposure and/or release of danger signals) associated with immunogenic cell death (ICD).

Therefore, compounds according to the present invention are for use in the prevention and/or treatment of an autophagy-related disease or condition in a mammalian subject, such as a human.

By “treatment” or “treating” is meant any treatment of a disease or disorder, in a mammal, including: preventing or protecting against the disease or disorder, that is, causing, the clinical symptoms of the disease not to develop; inhibiting the disease, that is, arresting or suppressing the development of clinical symptoms; and/or relieving the disease, that is, causing the regression of clinical symptoms. By “amelioration” is meant the prevention, reduction or palliation of a state, or improvement of the state of a subject; the amelioration of a stress is the counteracting of the negative aspects of a stress. Amelioration includes, but does not require complete recovery or complete prevention of a stress.

Preferred is a compound for use according to the present invention, wherein said autophagy-related disease or condition is selected from the group consisting of neurodegenerative diseases, Huntington's disease, Alzheimer's disease, Parkinson's disease, epilepsy, brain or other neuronal cancer, Wilson Disease, viral infection and related neuronal diseases, Niemann-Pick type C (NPC) disease, autoimmune diseases, multiple sclerosis, stroke, Wegener's granulomatosis, neuropathic pain, post-operative phantom limb pain or postherpetic neuralgia, ALS, spinal cord injury, and diseases and conditions of the CNS and/or neurons involving misfolded and/or nonfolded proteins as well as age-related forms of the diseases and conditions (such as neurodegenerative disease, cancer, and metabolic syndrome). Particularly preferred are ALS, Alzheimer's disease, and Parkinson's disease.

Cheon S Y, Kim H, Rubinsztein D C, and Lee J E. (in: Autophagy, Cellular Aging and Age-related Human Diseases. Exp Neurobiol 2019; 28:643-657. https://doi.org/10.5607/en.2019.28.6.643) disclose that during aging, cellular factors suggested as the cause of aging have been reported to be associated with progressively compromised autophagy. Dysfunctional autophagy may contribute to age-related diseases, such as neurodegenerative disease, cancer, and metabolic syndrome, in the elderly. Therefore, restoration of impaired autophagy to normal may help to prevent age-related disease and extend lifespan and longevity. They provide an overview of the mechanisms of autophagy underlying cellular aging and the consequent disease.

Similarly, Leidal, A. M., Levine, B. & Debnath, J. (in: Autophagy and the cell biology of age-related disease. Nat Cell Biol 20, 1338-1348 (2018). https://doi.org/10.1038/s41556-018-0235-8) review that autophagy declines with age and that impaired autophagy predisposes individuals to age-related diseases, whereas interventions that stimulate autophagy often promote longevity.

Also, Vaccaro Maria Ines, De Tata Vincenzo, and Gonzalez Claudio Daniel (in: Editorial: Autophagy in Endocrine-Metabolic Diseases Associated With Aging; Frontiers in Endocrinology, 11, 2020, pp 572, doi=10.3389/fendo.2020.00572) present a special issue containing a collection of 12 articles covering a broad range of key topics on the interplay of the different types of autophagy alterations with aging, endocrine-metabolic, and degenerative diseases.

Further preferred is the compound for use according to the present invention, wherein said prevention and/or treatment comprises a combination of at least two compounds for use according to the present invention, and/or a combination with at least one additional pharmaceutically active substance for said autophagy-related disease or condition.

It is to be understood that the present compound and/or a pharmaceutical composition comprising the present compound is for use to be administered to a human patient. The term “administering” means administration of a sole therapeutic agent or in combination with another therapeutic agent. It is thus envisaged that the pharmaceutical composition of the present invention are employed in co-therapy approaches, i.e. in co-administration with other medicaments or drugs and/or any other therapeutic agent which might be beneficial in the context of the methods of the present invention. Nevertheless, the other medicaments or drugs and/or any other therapeutic agent can be administered separately from the compound for use, if required, as long as they act in combination (i.e. directly and/or indirectly, preferably synergistically) with the present compound(s) (for use).

In another aspect of the compound for use according to the present invention the prevention and/or treatment further comprises detecting and/or monitoring in said subject a response of at least one autophagy-biomarker. The biomarker is preferably selected from the group consisting of BECN1, ATG8/LC3 family, including LC3A, LC3B, LC3C), LC3-II, ULK1, p62, NBR1, ATG5 and ATG7.

This monitoring usually is performed on a biologically sample taken from the mammalian subject, and comprises commonly known tests, for example antibody based, PCR based, and the like. The tests are repeated over time, and can be compared to control samples and/or samples taken earlier from the mammalian subject. The results help the attending physician to maintain or modify the course of a treatment, usually based on the severity of the clinical symptoms of the autophagy-related disease and/or condition as treated.

In another aspect thereof, the present invention provides methods for preventing and/or treating an autophagy-related disease and/or condition in a mammalian subject, such as a human, comprising administering to said mammal an effective amount of a compound or a pharmaceutical composition according to the present invention.

Preferred is the method according to the present invention, wherein said autophagy-related disease or condition is selected from the group consisting of neurodegenerative diseases, Huntington's disease, Alzheimer's disease, Parkinson's disease, epilepsy, brain or other neuronal cancer, Wilson Disease, viral infection and related neuronal diseases, Niemann-Pick type C (NPC) disease, autoimmune diseases, multiple sclerosis, stroke, Wegener's granulomatosis, neuropathic pain, post-operative phantom limb pain or postherpetic neuralgia, ALS, spinal cord injury, and diseases and conditions of the CNS and/or neurons involving misfolded and/or nonfolded proteins. Particularly preferred are ALS, Alzheimer's disease, and Parkinson's disease.

The dosage of the pharmaceutical composition to be administered according to the present invention can be appropriately selected according to the route of administration, the subject to be administered, the target disease and its severity, age, sex weight, individual differences and disease state. Dosage may be repeated several times a day.

According to the present invention, a mammalian subject can be preferably selected from a mouse, rat, cat, dog, rabbit, goat, sheep, horse, camel, lama, cow, monkey, a farm animal, a sport animal, and a pet, and a human.

In addition to the aforementioned compounds of the invention, the pharmaceutical composition as administered can contain two or more compounds according to the present invention and also other therapeutically active substances. In the method, the compound for use can be provided and/or is administered as a suitable pharmaceutical composition as discussed above. The compounds can be administered alone or in combination with other active compounds—for example with medicaments already known for the treatment of the aforementioned conditions and/or diseases, whereby in the latter case a favorable additive, amplifying or preferably synergistically effect is noticed.

In another aspect of the method according to the present invention the prevention and/or treatment further comprises detecting and/or monitoring in said subject a response of at least one autophagy-biomarker. The biomarker is preferably selected from the group of BECN1, and the ATG8/LC3 family, including LC3A, LC3B, LC3C), LC3-II, ULK1, p62, NBR1, ATG5 and ATG7.

This monitoring usually is performed on a biologically sample taken from the mammalian subject, and comprises commonly known tests, for example antibody based, PCR based, and the like. The tests are repeated over time, and can be compared to control samples and/or samples taken earlier from the mammalian subject. The results help the attending physician to maintain or modify the course of a treatment, usually based on the severity of the clinical symptoms of the autophagy-related disease and/or condition as treated.

It was surprisingly found that the compounds according to Formula I, as an autophagy inducer, were effective and exhibits many advantages compared to other autophagy-related treatment options, particularly as a combination treatment. In comparison with prior art compounds as tested (including data not shown), an improvement of the present invention lies in the unexpected observation that the compounds described herein are highly effective autophagy inducers (see Examples below).

The present invention will now be described further in the following examples, nevertheless, without being limited thereto. For the purposes of the present invention, all references as cited herein are incorporated by reference in their entireties.

### EXAMPLES

The following examples have been performed in part using preferred compounds as disclosed. Nevertheless, it should be understood that the present invention is nor in any way limited thereto, and the person of skill is readily able to adjust the conditions as described to other compounds according to the present invention.

Compounds of the present invention were synthesized from commercially available starting materials, in preferably by following the relevant general experimental procedures as given below (general experimental procedures 1-4).

### Abbreviations Used

Boc tertiary-butoxycarbonyl
IBu tertiary butyl
DCM dichloromethane
DIPEA N,N-Diisopropylethylamine
DMF dimethylformamide
DMSO dimethylsulfoxide
dppf 1, 1′-bis(diphenylphosphino) ferrocene
eq equivalents
ESI electrospray ionisation
Hz hertz
H hours
HATU 1-[Bis(dimethylamino)methylene]-1H-1,2,3-triazolo[4,5-b]pyridinium 3-oxide hexafluorophosphate
HPLC high performance liquid chromatography
J coupling constant in Hz
LCMS liquid chromatography-mass spectrometry min minutes
NMR nuclear magnetic resonance
N normal (equivalents per litre)
Ph phenyl
RT retention time
rt room temperature
SFC supercritical fluid chromatography
THF tetrahydrofuran
TLC thin-layer chromatography
vol volumes
X-Phos 2-Dicyclohexylphosphin-2′,4′,6′-triisopropylbiphenyl
X-Phos Pd G2 Second generation XPhos Precatalyst (X-Phos aminobiphenyl palladium chloride precatalyst); Chloro(2-dicyclohexylphosphino-2′,4′,6′-triisopropyl-1,1′-biphenyl) [2-(2′-amino-1,1′-biphenyl)]palladium (II)

### General Experimental Procedure 1

### Schematic Overview of Synthesis:

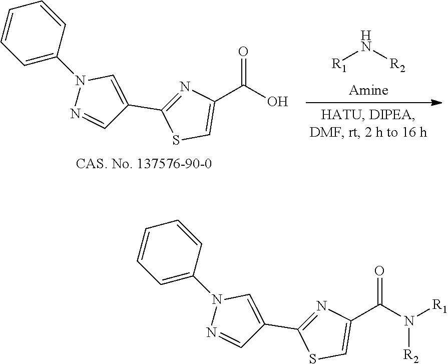

### Typical General Conditions

To a solution of 2-(1-phenyl-1H-pyrazol-4-yl) thiazole-4-carboxylic acid (CAS.No.137576-90-0, 1.0 eq) in DMF (10 vol) at room temperature, HATU (1.5 eq) and DIPEA (3.0 eq) were added. The reaction mixture was stirred at rt for 15-30 min, then the amine derivative (1.0 eq) was added to reaction mixture at room temperature. The reaction mixture was stirred at rt for 2 h to 16 h (the progress of the reaction was monitored by TLC and LCMS analysis). After completion of the reaction, the reaction mixture was poured into cold water (10 vol) and extracted with ethyl acetate (3×5 vol). The combined organic fractions were washed with cold water 3-4 times, dried over sodium sulfate and concentrated under reduced pressure. The crude material was purified by flash column chromatography (CombiFlash® system) to provide the target compound.

### General Experimental Procedure 2

### Schematic Overview of Synthesis:

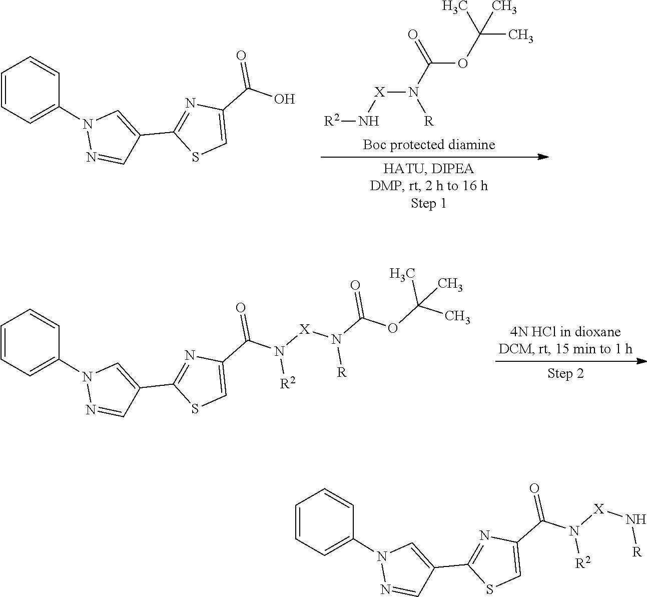

### Typical General Conditions

### Step 1—Synthesis of Boc-Protected Intermediate

To a solution of 2-(1-phenyl-1H-pyrazol-4-yl) thiazole-4-carboxylic acid (CAS. No. 137576-90-0, 1.0 eq) in DMF (10 vol) at rt, HATU (1.5 eq) and DIPEA (3.0 eq) were added. The reaction mixture was stirred for 15-20 min, then the Boc-protected amine derivative (1.0 eq) was added to reaction mixture at rt. The reaction mixture was stirred at rt for 2 h to 16 h (the progress of the reaction was monitored by TLC and LCMS analysis). After completion of the reaction, the reaction mixture was poured into cold water (10 vol) and extracted with ethyl acetate (3×5 vol). The combined organic fractions were washed with cold water 3-4 times, dried over sodium sulfate and concentrated under reduced pressure. The crude material was purified by flash column chromatography (CombiFlash® system) to provide the Boc-protected intermediate.

### Step 2—Deprotection

To a stirred solution of the Boc-protected intermediate (1.0 eq) in DCM (10 vol) at 0° C. or rt, 4 N HCl in dioxane was added. The resulting reaction mixture was stirred at rt for 15 min to 1 h (the progress of the reaction was monitored by TLC and LCMS analysis). After completion of reaction, the reaction mixture was concentrated under reduced pressure and triturated (typically with diethyl ether or DCM) to provide the target compound.

### General Experimental Procedure 3

### Schematic Overview of Synthesis (X=Unspecified Heteroatom; R Various):

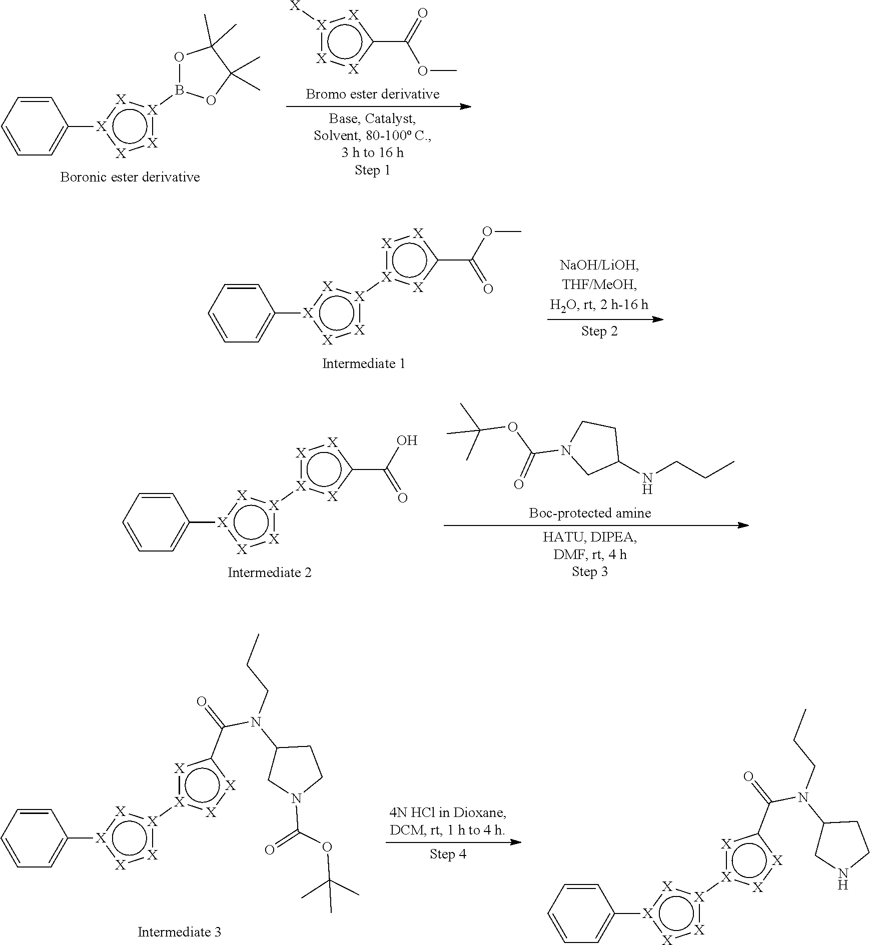

### Typical General Conditions

### Step 1—Cross-Coupling

To a stirred solution of the Boronic ester derivative (1.0 eq) and the Bromo ester derivative (1.2 eq) in a suitable solvent (DMF or 1,4-dioxane:water (9:1, 10 vol) at rt, a suitable base (potassium phosphate or potassium carbonate or sodium carbonate; 2.0 to 3.0 eq) was added. The reaction mixture was degassed with nitrogen gas for 10 min. A suitable palladium catalyst (Pd(PPh₃)₄ or PdCl₂(dppf).DCM or X-Phos Pd G2 or (t-Bu₃P)₂Pd; 0.1 eq) was added. The reaction mixture was again degassed with nitrogen gas for 10 min and then heated at 80° C.-100° C. for 3 h to 16 h (the progress of the reaction was monitored by TLC and LCMS analysis). After completion of the reaction, the reaction mixture was diluted with water (10 vol) and extracted with ethyl acetate (3×5 vol). The combined organic fractions were dried over anhydrous sodium sulfate and concentrated under reduced pressure. The crude material was purified by flash column chromatography (CombiFlash® system) to provide Intermediate 1.

### Step 2—Ester Hydrolysis

To a stirred solution of Intermediate 1 (1.0 eq) in a suitable solvent (typically THF (10 vol) or MeOH (10 vol) or EtOH (10 vol)) at rt, a suitable base (typically LiOH (8.0 eq) in water (10 vol) or NaOH (5.0 eq) in water (10 vol)) was added. The reaction mixture was stirred at rt for 2 h to 16 h (the progress of the reaction was monitored by TLC and LCMS analysis). After completion of reaction, the reaction mixture was distilled out and extracted with ethyl acetate (2×5 vol). The aqueous layer was acidified (typically with IN HCl or a saturated solution of citric acid) and extracted with ethyl acetate (3×10 vol). The combined organic fractions were dried over sodium sulfate and concentrated under reduced pressure to provide Acid Intermediate 2.

### General Experimental Procedure 4

### Schematic Overview of Synthesis (R=Aryl or Heteroaryl [Unsubstituted and Substituted] or Alkyl):

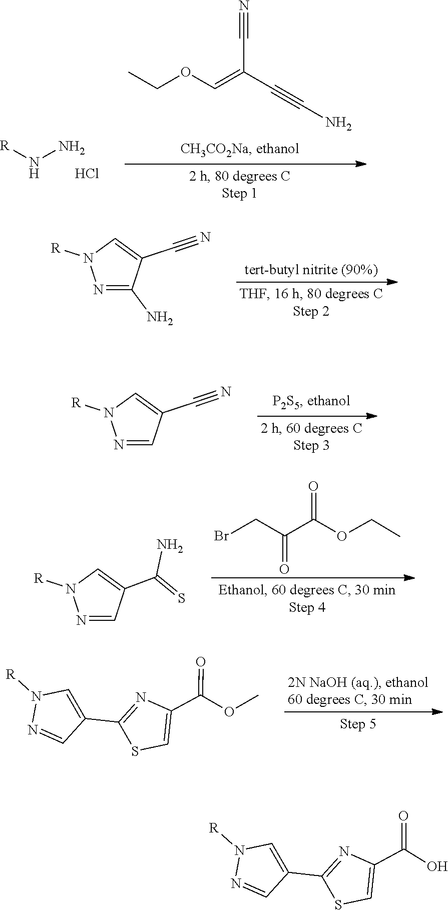

### Typical General Conditions

### Step-1: Synthesis of 5-amino-1H-pyrazole-4-carbonitrile Intermediate

To a stirred solution of the appropriate hydrazine hydrochloride (20.69 mmol) in ethanol (50 mL) at rt, sodium acetate (3.39 g, 41.39 mmol) and 2-(ethoxymethylene) malononitrile (2.52 g, 20.69 mmol) were added. The reaction mixture was refluxed at 80° C. for 2 h (the progress of reaction was monitored by TLC and LCMS analysis). After completion of the reaction, the reaction mixture was concentrated under reduced pressure. The residual material was washed with cold water (50 mL) and extracted with ethyl acetate (3×100 mL). The combined organic fractions were dried over anhydrous sodium sulfate and concentrated under reduced pressure to provide the crude 5-amino-1-aryl-1H-pyrazole-4-carbonitrile.

### Step-2: Synthesis of 1H-pyrazole-4-carbonitrile Intermediate

To a stirred solution of the crude 5-amino-1-aryl-1H-pyrazole-4-carbonitrile (18.24 mmol) in THF (50 mL) at rt, tert-butyl nitrite (90%) (4.13 g, 36.49 mmol) was added. The reaction mixture was refluxed at 80° C. for 16 h (the progress of the reaction was monitored by TLC and LCMS analysis). After completion of reaction, the reaction mixture was concentrated under reduced pressure. The residual material was purified by flash column chromatography to provide the 1H-pyrazole-4-carbonitrile.

### Step-3: Synthesis of 1H-pyrazole-4-carbothioamide Intermediate

To a stirred solution of the 1H-pyrazole-4-carbonitrile (15.5 mmol) in ethanol (37 mL) at rt, phosphorus pentasulfide (6.93 g, 31.1 mmol) was added. The reaction mixture was refluxed at 60° C. for 2 h (the progress of the reaction was monitored by TLC and LCMS analysis). After completion of the reaction, the reaction mixture was distilled under vacuum. The crude reaction mixture was washed with sodium bicarbonate solution (100 mL) until neutralization of pH and extracted with ethyl acetate (3×150 mL). The combined organic fractions were dried over sodium sulfate, concentrated under reduced pressure and triturated with diethyl ether to afford the crude 1H-pyrazole-4-carbothioamide.

### Step-4: Synthesis of ethyl 2-aryl-1H-pyrazol-4-yl) thiazole-4-carboxylate

To a stirred solution of the 1H-pyrazole-4-carbothioamide (14.70 mmol) in ethanol (50 mL) at rt, ethyl 3-bromo-2-oxopropanoate (3.14 g, 16.17 mmol) was added. The reaction mixture was refluxed at 60° C. for 30 min (the progress of the reaction was monitored by TLC and LCMS analysis). After completion of the reaction, the reaction mixture was distilled under vacuum to provide the ethyl 2-aryl-1H-pyrazol-4-yl) thiazole-4-carboxylate.

### Step-5: Synthesis of 2-aryl-1H-pyrazol-4-yl) thiazole-4-carboxylic acid

To a stirred solution of the ethyl 2-aryl-1H-pyrazol-4-yl) thiazole-4-carboxylate (14.4 mmol) in ethanol (60 mL) at rt, 2 N NaOH solution (21.62 mL) was added. The reaction mixture was heated at 60° C. for 30 minutes (the progress of the reaction was monitored by TLC and LCMS analysis). After completion of the reaction, the reaction mixture was poured into water (50 mL) and extracted with ethyl acetate (3×50 mL). The aqueous layer was separated out and acidified with 2 M HCl solution to generated a precipitate, which was isolated by filtration and dried under reduced pressure to provide the 2-aryl-1H-pyrazol-4-yl) thiazole-4 carboxylic acid.

### General Experimental Procedure 5

### 5A: Schematic Overview of Synthesis (X=Unspecified Heteroatom)

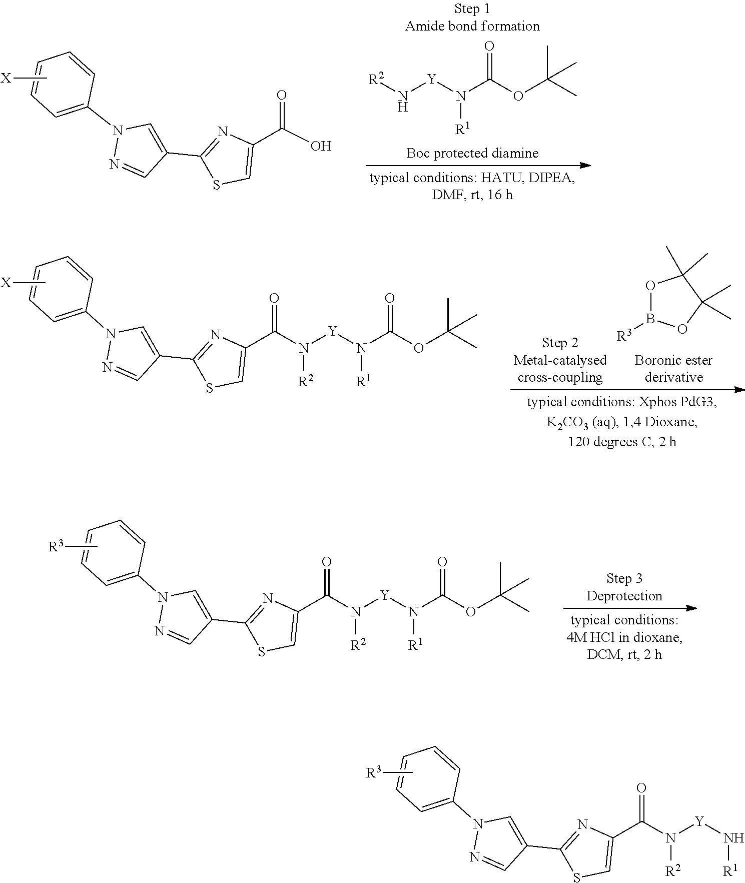

### 5B: Schematic Overview of Synthesis (X=Unspecified Heteroatom)

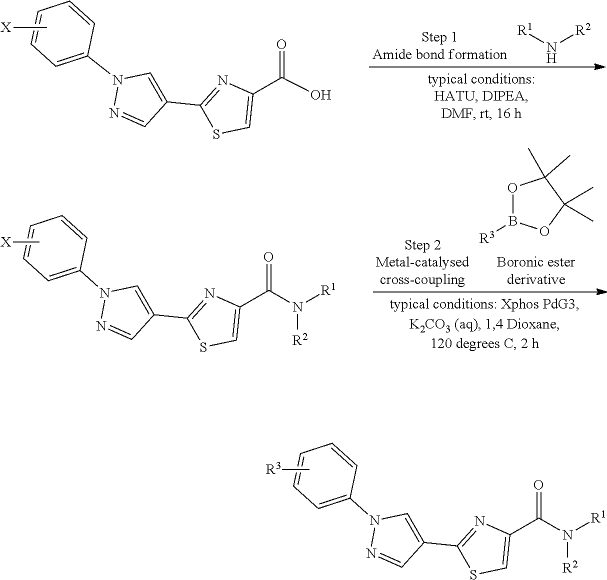

### Overview of Synthetic Route Towards Compound 302

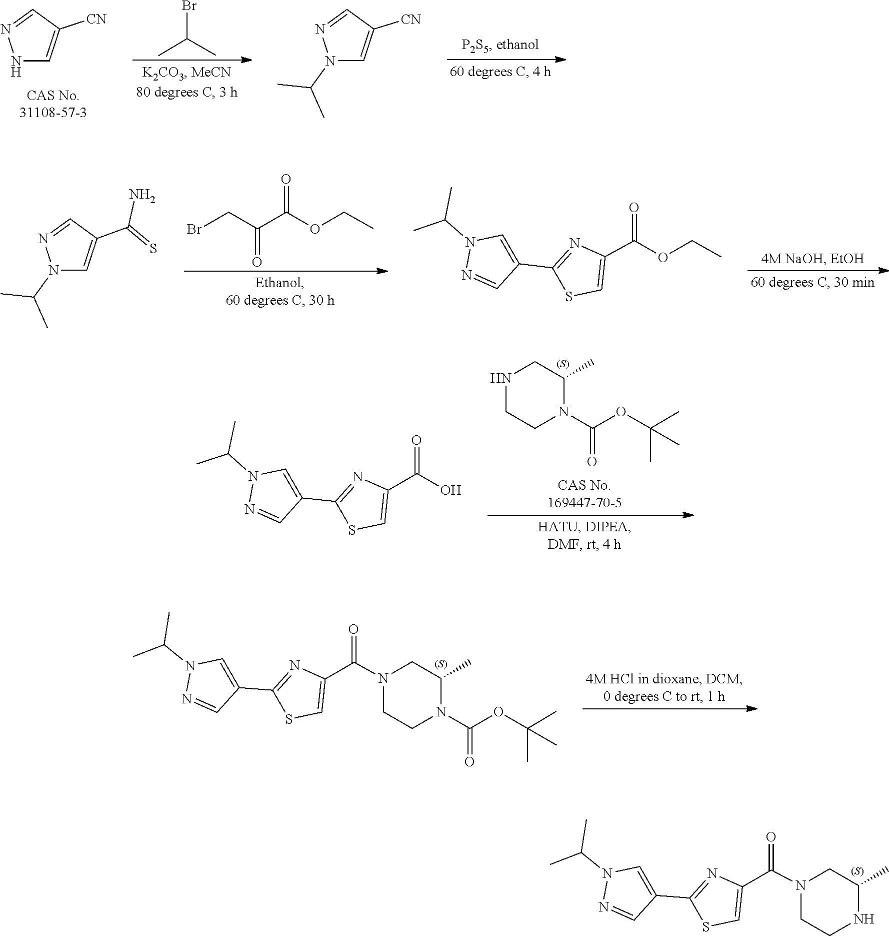

### Full Synthetic Details for a Representative Set of Compounds

### 1. Synthesis of N-(1-ethylpiperidin-4-yl)-N-methyl-2-(1-phenyl-1H-pyrazol-4-yl) thiazole-4-carboxamide (Compound 196)

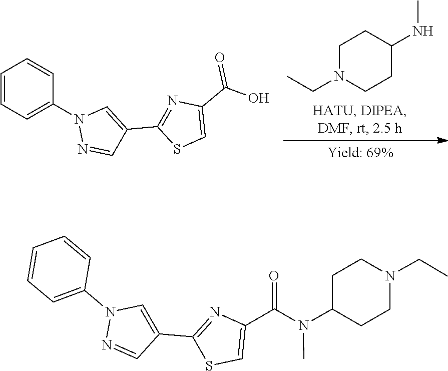

Prepared according to General experimental procedure 1 using 2-(1-phenyl-1H-pyrazol-4-yl) thiazole-4-carboxylic acid (0.050 g, 0.18 mmol), HATU (0.105 g, 0.27 mmol) and DIPEA (0.1 mL, 0.55 mmol) in DMF (1.8 mL), stirred for 30 min prior to the addition of 1-ethyl-N-methylpiperidin-4-amine (0.026 g, 0.18 mmol). The reaction mixture was stirred at rt for 2.5 h. The crude material was purified by flash column chromatography (CombiFlash® system; 4-5% MeOH/DCM) to provide N-(1-ethylpiperidin-4-yl)-N-methyl-2-(1-phenyl-1H-pyrazol-4-yl) thiazole-4-carboxamide (Compound 196:0.043 g, 69% yield) as an pale yellow solid.

LCMS [ESI, M+1]: 396.12 (RT: 1.280 min, Purity: 100%),

HPLC: RT: 6.816 min, Purity: 100%

¹H NMR (400 MHZ, d₆-DMSO) δ 9.12 (s, 1H), 8.17 (d, J=22.9 Hz, 1H), 7.91-7.84 (m, 3H), 7.47 (t, J=7.8 Hz, 2H), 7.30 (t, J=7.2 Hz, 1H), 4.26 (m, 1H), 3.73 (m, 1H), 2.97 (m, 2H), 2.88 (s, 3H), 1.72 (m, 7H), 0.89 (t, J=18 Hz, 3H).

### 2. Synthesis of 4-(2-(1-phenyl-1H-pyrazol-4-yl) thiazole-4-carbonyl) piperazine-1-sulfonamide (Compound 105)

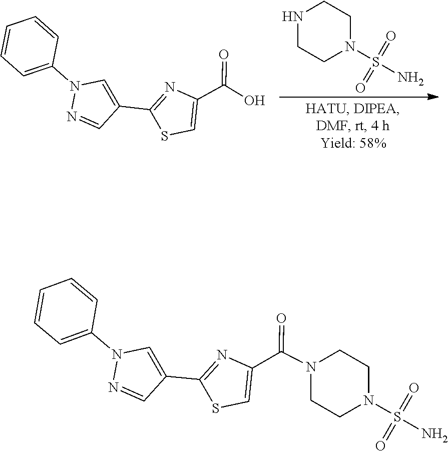 [SMILES: O=C(O)c1csc(-c2cnn(-c3ccccc3)c2)n1 ; NS(=O)(=O)N1CCNCC1 ; NS(=O)(=O)N1CCN(C(=O)c2csc(-c3cnn(-c4ccccc4)c3)n2)CC1]

Prepared according to general procedure 1 using 2-(1-phenyl-1H-pyrazol-4-yl) thiazole-4-carboxylic acid (0.100 g, 0.36 mmol), HATU (0.210 g, 0.55 mmol) and DIPEA (0.143 g, 7.74 mmol) in DMF (1 mL), stirred for 15 min prior to the addition of piperazine-1-sulfonamide (0.480 g, 1.10 mmol. The reaction mixture was stirred at rt for 4 h. The following modification to the general procedure were used: after completion of the reaction, the reaction mixture was poured into ice cold water to obtain a solid precipitate. The solid material was isolated by filtration and washed with ice cold water (20 mL) and hexane (30 mL). The solid material was triturated with diethyl ether to provide 4-(2-(1-phenyl-1H-pyrazol-4-yl) thiazole-4-carbonyl) piperazine-1-sulfonamide (Compound 105: 0.090 g, 58% yield) as a light brown solid.

LCMS [ESI, M+1]: 418.9 (RT: 1.576 min, Purity: 89.38%),

HPLC: RT: 6.139 min, Purity: 95.57%,

¹H NMR (400 MHZ, d₆-DMSO): δ 9.21 (s, 1H), 8.30 (s, 1H), 8.10 (s, 1H), 7.94 (d, J=7.8 Hz, 2H), 7.55 (t, J=7.9 Hz, 2H), 7.38 (t, J=7.4 Hz, 1H), 6.89 (s, 2H), 3.81 (m, 4H), 3.04 (m, 4H).

### 3. Synthesis of N-(1-ethylpiperidin-4-yl)-2-(1-phenyl-1H-pyrazol-4-yl) thiazole-4-carboxamide (Compound 185)

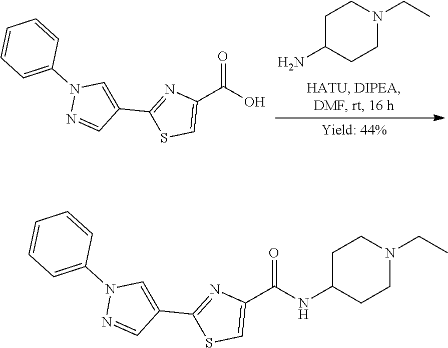

Prepared by general procedure 1 using 2-(1-phenyl-1H-pyrazol-4-yl) thiazole-4-carboxylic acid (0.050 g, 0.184 mmol) HATU (0.105 g, 0.276 mmol) and DIPEA (0.1 mL, 0.552 mmol) in DMF (1.8 mL, 36V), stirred for 20 min prior to the addition of 1-ethylpiperidin-4-amine (0.023 g, 0.184 mmol). The reaction mixture was stirred at rt for 16 h. The crude material was purified by flash column chromatography (CombiFlash® system; 80% ethyl acetate/hexane) to provide N-(1-ethylpiperidin-4-yl)-2-(1-phenyl-1H-pyrazol-4-yl) thiazole-4-carboxamide (Compound 185:0.031 g, 44. % yield) as a white solid.

LCMS [ESI, M+1]: 382.1 (RT: 1.29 min, Purity: 99.52%),

HPLC: RT: 7.137 min, Purity: 99.29%

¹H NMR (400 MHZ, d₆-DMSO): δ 9.14 (s, 1H), 8.89 (s, 1H), 8.21 (d, J=35.6 Hz, 2H), 7.86 (d, J=7.9 Hz, 2H), 7.47 (t, J=7.6 Hz, 2H), 7.31 (d, J=7.2 Hz, 1H), 4.06 (m, 1H), 3.51 (d, J=46.9 Hz, 2H), 3.01 (m, 4H), 1.95 (m, 2H), 1.80 (m, 2H), 1.14 (t, J=6.4 Hz, 3H).

### 4. Synthesis of(S)-(3-methylpiperazin-1-yl) (2-(1-phenyl-1H-pyrazol-4-yl) thiazol-4-yl) methanone hydrochloride (Compound 157)

### Overall Synthetic Scheme

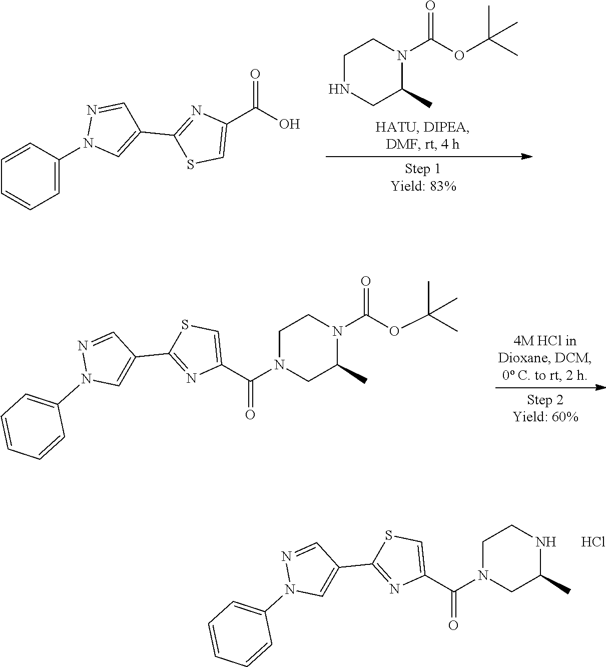 [SMILES: O=C(O)c1csc(-c2cnn(-c3ccccc3)c2)n1 ; C[C@H]1CNCCN1C(=O)OC(C)(C)C ; C[C@H]1CN(C(=O)c2csc(-c3cnn(-c4ccccc4)c3)n2)CCN1C(=O)OC(C)(C)C ; C[C@H]1CN(C(=O)c2csc(-c3cnn(-c4ccccc4)c3)n2)CCN1 ; Cl]

### Synthetic Procedure

In step 1 2—(1-phenyl-1H-pyrazol-4-yl) thiazole-4-carboxylic acid (0.050 g, 0.18 mmol) and(S)-2-methylpiperazine-1-carboxylate (0.039 g, 0.18 mmol) were used. The solution of -(1-phenyl-1H-pyrazol-4-yl) thiazole-4-carboxylic acid (0.050 g, 0.18 mmol), HATU (0.105 g, 0.27 mmol) and DIPEA (0.071 g, 0.55 mmol) in in DMF (0.5 mL) was stirred at rt for 15 min prior to the addition of(S)-2-methylpiperazine-1-carboxylate (0.039 g, 0.18 mmol). The reaction mixture was stirred at rt for 4 h. The crude material was purified by flash column chromatography (CombiFlash® system; 0-45% ethyl acetate/hexane) to provide tert-butyl(S)-2-methyl-4-(2-(1-phenyl-1H-pyrazol-4-yl) thiazole-4-carbonyl) piperazine-1-carboxylate (0.069 g, 83% yield) as an off-white solid.

LCMS [ESI, M+1]: 398.1 [M-56] (RT: 2.232 min, Purity: 97.00%),

¹H NMR (400 MHZ, d₆-DMSO): 9.23 (s, 1H), 8.32 (s, 1H), 8.08 (d, J=40.6 Hz, 1H), 7.98 (d, J=7.9 Hz, 2H), 7.58 (t, J=7.8 Hz, 2H), 7.41 (t, J=7.3 Hz, 1H), 4.45-4.15 (m, 4H), 3.82 (s, 1H), 3.14 (d, J=28.7 Hz, 2H), 1.44 (s, 9H), 1.12 (s, 3H).

In step 2 tert-butyl(S)-2-methyl-4-(2-(1-phenyl-1H-pyrazol-4-yl) thiazole-4-carbonyl) piperazine-1-carboxylate (0.07 g, 0.20 mmol) in DCM (0.7 mL) was used. The solution was cooled to 0° C. prior to the addition of 4 M HCl in Dioxane (0.7 mL, 5V). The reaction mixture was stirred at rt for 2 h. The crude material was triturated with diethyl ether to provide(S)-(3-methylpiperazin-1-yl) (2-(1-phenyl-1H-pyrazol-4-yl) thiazol-4-yl) methanone hydrochloride (Compound 157:0.032 g, 60% yield) as an off-white solid.

LCMS [ESI, M+1]: 354.1 (RT: 1.497 min, Purity: 98.55%),

HPLC Purity: RT: 4.373 min, Purity: 99.24%,

¹H NMR (400 MHZ, d₆-DMSO): δ 9.22 (s, 1H), 8.30 (s, 1H), 8.17 (s, 1H), 7.94 (d, J=8.0 Hz, 2H), 7.52 (dd, J=37.0, 29.2 Hz, 2H), 7.38 (t, J=7.6 Hz, 1H), 4.47 (d, J=11.6 Hz, 2H), 3.38 (d, J=6.9 Hz, 3H), 3.14 (d, J=21.2 Hz, 2H), 1.28 (d, J=16.8 Hz, 3H).

### 5. Synthesis of (2S)-1-[2-(1-phenyl-1H-pyrazol-4-yl)-1,3-thiazole-4-carbonyl]pyrrolidine-2-carboxylic acid (Compound 60)

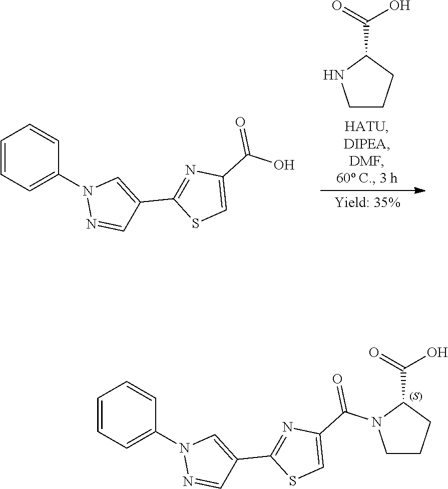

Prepared according to general experimental procedure 1 using L-proline (0.169 g, 1.47 mmol). The reaction mixture was stirred at 60° C. for 3 h. A modified purification procedure was used: the crude material was purified by preparative HPLC (0.05% HCl in water/ACN as the mobile phase) to provide (2S)-1-[2-(1-phenyl-1H-pyrazol-4-yl)-1,3-thiazole-4-carbonyl]pyrrolidine-2-carboxylic acid (Compound 60:0.190 g, 35% yield) as a white solid.

LCMS [ESI, M+1]: 369.0 (RT: 1.617 min, Purity: 99.36%),

HPLC: RT: 6.319 min, Purity: 98.81%

¹H NMR (400 MHZ, CD₃OD): δ 8.83 (s, 1H), 8.25 (s, 1H), 8.20 (s, 1H), 7.84 (d, J=8.8 Hz, 2H), 7.53 (t, J=8.0 Hz, 2H), 7.40 (dd, J=10.7, 4.2 Hz, 1H), 5.36 (dd, J=8.8, 2.7 Hz, 1H), 3.86-3.76 (m, 2H), 2.36-2.26 (m, 2H), 2.14-1.92 (m, 2H).

TABLE 1
Compounds 1-304 with chemical name, LCMS data and
experimental procedure

| | | | Observed | | |
|---|---|---|---|---|---|
| Comp. | | | MW in | Salt | Exp. Proc. |
| No. | Chemical Name | MW | LCMS | Form | No. |
| 1 | N-(1-ethylpiperidin-4-yl)-N- | 377 | 378 | Free base | 3 |
| | methyl-5-(1-phenyl-1H-pyrazol-4- | | | | |
| | yl)-1H-pyrrole-2-carboxamide | | | | |
| 2 | (3S)-3-methyl-1-[4-(5-phenyl- | 370 | 371 | HCl salt | 3 |
| | 1,3,4-thiadiazol-2-yl)thiophene- | | | | |
| | 2-carbonyl]piperazine | | | | |
| 3 | (3S)-3-methyl-1-[4-(1-phenyl- | 335 | 336 | HCl salt | 3 |
| | 1H-pyrazol-4-yl)-1H-pyrrole-2- | | | | |
| | carbonyl]piperazine | | | | |
| 4 | (3S)-3-methyl-1-[5-(1-phenyl- | 352 | 353 | HCl salt | 3 |
| | 1H-pyrazol-4-yl)thiophene-3- | | | | |
| | carbonyl]piperazine | | | | |
| 7 | (3S)-3-methyl-1-[2-(5- | 369 | 370 | HCl salt | 3 |
| | phenylthiophen-2-yl)-1,3- | | | | |
| | thiazole-4-carbonyl]piperazine | | | | |
| 10 | N-methyl-N-(oxan-4-yl)-5-(1- | 351 | 352 | Free base | 3 |
| | phenyl-1H-pyrazol-4-yl)furan-2- | | | | |
| | carboxamide | | | | |
| 13 | (3S)-3-methyl-1-[2-(2-phenyl- | 354 | 355 | HCl salt | 3 |
| | 1,3-oxazol-5-yl)-1,3-thiazole-4- | | | | |
| | carbonyl]piperazine | | | | |
| 15 | N-methyl-N-(oxan-4-yl)-4-(1- | 367 | 368 | Free base | 3 |
| | phenyl-1H-pyrazol-4- | | | | |
| | yl)thiophene-2-carboxamide | | | | |
| 19 | N-methyl-N-(oxan-4-yl)-5-(1- | 350 | 351 | Free base | 3 |
| | phenyl-1H-pyrazol-4-yl)-1H- | | | | |
| | pyrrole-2-carboxamide | | | | |
| 24 | (3S)-3-methyl-1-(2-{1-[3- | 437 | 438 | HCl salt | 4 |
| | (trifluoromethoxy)phenyl]-1H- | | | | |
| | pyrazol-4-yl}-1,3-thiazole-4- | | | | |
| | carbonyl)piperazine | | | | |
| 27 | (3S)-3-methyl-1-{2-[1-(3- | 367 | 368 | HCl salt | 4 |
| | methylphenyl)-1H-pyrazol-4-yl]- | | | | |
| | 1,3-thiazole-4-carbonyl}piperazine | | | | |
| 30 | (3S)-1-{2-[1-(3-chlorophenyl)- | 387 | 388 | HCl salt | 4 |
| | 1H-pyrazol-4-yl]-1,3-thiazole-4- | | | | |
| | carbonyl}-3-methylpiperazine | | | | |
| 33 | (3S)-3-methyl-1-{2-[1-(pyridin- | 354 | 355 | HCl salt | 4 |
| | 2-yl)-1H-pyrazol-4-yl]-1,3- | | | | |
| | thiazole-4-carbonyl}piperazine | | | | |
| 34 | (3S)-1-{2-[1-(4-fluorophenyl)- | 371 | 372 | HCl salt | 4 |
| | 1H-pyrazol-4-yl]-1,3-thiazole-4- | | | | |
| | carbonyl}-3-methylpiperazine | | | | |
| 37 | (3S)-3-methyl-1-(2-{1-[4- | 437 | 438 | HCl salt | 4 |
| | (trifluoromethoxy)phenyl]-1H- | | | | |
| | pyrazol-4-yl}-1,3-thiazole-4- | | | | |
| | carbonyl)piperazine | | | | |
| 38 | (3S)-3-methyl-1-(2-{1-[3- | 421 | 422 | HCl salt | 4 |
| | (trifluoromethyl)phenyl]-1H- | | | | |
| | pyrazol-4-yl}-1,3-thiazole-4- | | | | |
| | carbonyl)piperazine | | | | |
| 39 | (3S)-1-{2-[1-(4-chlorophenyl)-1H- | 388 | 388 | HCl salt | 4 |
| | pyrazol-4-yl]-1,3-thiazole-4- | | | | |
| | carbonyl}-3-methylpiperazine | | | | |
| 55 | (2R)-1-[2-(1-phenyl-1H-pyrazol- | 367 | 369 | Free base | see full |
| | 4-yl)-1,3-thiazole-4- | | | | experimental |
| | carbonyl]pyrrolidine-2- | | | | |
| | carboxylic acid | | | | |
| 60 | (2S)-1-[2-(1-phenyl-1H-pyrazol-4- | 368 | 369 | Free base | see full |
| | yl)-1,3-thiazole-4- | | | | experimental |
| | carbonyl]pyrrolidine-2- | | | | |
| | carboxylic acid | | | | |
| 62 | (3S)-1-{2-[1-(3-methoxyphenyl)- | 383 | 384 | HCl salt | 4 |
| | 1H-pyrazol-4-yl]-1,3-thiazole-4- | | | | |
| | carbonyl}-3-methylpiperazine | | | | |
| 63 | (3S)-3-methyl-1-{2-[1-(4- | 367 | 368 | HCl salt | 4 |
| | methylphenyl)-1H-pyrazol-4-yl]- | | | | |
| | 1,3-thiazole-4- | | | | |
| | carbonyl}piperazine | | | | |
| 67 | (3S)-3-methyl-1-[5-(1-phenyl- | 335 | 336 | HCl salt | 3 |
| | 1H-pyrazol-4-yl)-1H-pyrrole-2- | | | | |
| | carbonyl]piperazine | | | | |
| 72 | (3S)-3-methyl-1-(2-{1-[2- | 421 | 422 | HCl salt | 4 |
| | (trifluoromethyl)phenyl]-1H- | | | | |
| | pyrazol-4-yl}-1,3-thiazole-4- | | | | |
| | carbonyl)piperazine | | | | |
| 75 | (3S)-3-methyl-1-(2-{1-[4- | 421 | 422 | HCl salt | 4 |
| | (trifluoromethyl)phenyl]-1H- | | | | |
| | pyrazol-4-yl}-1,3-thiazole-4- | | | | |
| | carbonyl)piperazine | | | | |
| 79 | (3S)-1-{2-[1-(2-chlorophenyl)-1H- | 388 | 388 | HCl salt | 4 |
| | pyrazol-4-yl]-1,3-thiazole-4- | | | | |
| | carbonyl}-3-methylpiperazine | | | | |
| 85 | (3S)-3-methyl-1-{2-[1-(2- | 367 | 368 | HCl salt | 4 |
| | methylphenyl)-1H-pyrazol-4-yl]- | | | | |
| | 1,3-thiazole-4-carbonyl}piperazine | | | | |
| 96 | (3S)-1-{2-[1-(2-fluorophenyl)- | 371 | 372 | HCl salt | 4 |
| | 1H-pyrazol-4-yl]-1,3-thiazole-4- | | | | |
| | carbonyl}-3-methylpiperazine | | | | |
| 102 | (3S)-3-methyl-1-[5-(1-phenyl-1H- | 336 | 337 | HCl salt | 3 |
| | pyrazol-4-yl)furan-2- | | | | |
| | carbonyl]piperazine | | | | |
| 103 | N-(1-ethylpiperidin-4-yl)-4-(1- | 381 | 381 | HCl salt | 3 |
| | phenyl-1H-pyrazol-4- | | | | |
| | yl)thiophene-2-carboxamide | | | | |
| 105 | 4-[2-(1-phenyl-1H-pyrazol-4-yl)- | 418 | 419 | Free base | see full |
| | 1,3-thiazole-4- | | | | experimental |
| | carbonyl]piperazine-1- | | | | |
| | sulfonamide | | | | |
| 109 | N-(1-ethylpiperidin-4-yl)-5-(1- | 364 | 365 | HCl salt | 3 |
| | phenyl-1H-pyrazol-4-yl)furan-2- | | | | |
| | carboxamide | | | | |
| 113 | (3S)-3-methyl-1-[4-(1-phenyl-1H- | 352 | 353 | HCl salt | 3 |
| | pyrazol-4-yl)thiophene-2- | | | | |
| | carbonyl]piperazine | | | | |
| 118 | (3R)-3-methyl-1-[2-(1-phenyl-1H- | 353 | 354 | HCl salt | 2 |
| | pyrazol-4-yl)-1,3-thiazole-4- | | | | |
| | carbonyl]piperazine | | | | |
| 125 | methyl 4-hydroxy-1-[2-(1-phenyl- | 398 | 399 | Free base | 1 |
| | 1H-pyrazol-4-yl)-1,3-thiazole-4- | | | | |
| | carbonyl]pyrrolidine- | | | | |
| | 2-carboxylate | | | | |
| 134 | N,N-diethyl-1-[2-(1-phenyl-1H- | 396 | 396 | Free base | 1 |
| | pyrazol-4-yl)-1,3-thiazole-4- | | | | |
| | carbonyl]pyrrolidin-3-amine | | | | |
| 138 | N-(1-methylpiperidin-4-yl)-2-(1- | 367 | 368 | Free base | 1 |
| | phenyl-1H-pyrazol-4-yl)-1,3- | | | | |
| | thiazole-4-carboxamide | | | | |
| 144 | 1-[2-(1-phenyl-1H-pyrazol-4-yl)- | 367 | 369 | Free base | 1 |
| | 1,3-thiazole-4- | | | | |
| | carbonyl]pyrrolidine-2- | | | | |
| | carboxylic acid | | | | |
| 147 | 2-(1-phenyl-1H-pyrazol-4-yl)-N- | 368 | 369 | Free base | 1 |
| | [(1r,4r)-4-hydroxycyclohexyl]-1,3- | | | | |
| | thiazole-4-carboxamide | | | | |
| 148 | 2-(1-phenyl-1H-pyrazol-4-yl)-N- | 407 | 407 | Free base | 1 |
| | {2,6,6- | | | | |
| | trimethylbicyclo[3.1.1]heptan-3- | | | | |
| | yl}-1,3-thiazole-4-carboxamide | | | | |
| 151 | N-cyclohexyl-2-(1-phenyl-1H- | 395 | 395 | Free base | 1 |
| | pyrazol-4-yl)-N-propyl-1,3- | | | | |
| | thiazole-4-carboxamide | | | | |
| 155 | N-cyclohexyl-N-[2- | 424 | 424 | Free base | 1 |
| | (dimethylamino)ethyl]-2-(1- | | | | |
| | phenyl-1H-pyrazol-4-yl)-1,3- | | | | |
| | thiazole-4-carboxamide | | | | |
| 157 | (3S)-3-methyl-1-[2-(1-phenyl- | 353 | 354 | HCl salt | 2 |
| | 1H-pyrazol-4-yl)-1,3-thiazole-4- | | | | |
| | carbonyl]piperazine | | | | |
| 164 | N-methyl-1-[2-(1-phenyl-1H- | 410 | 410 | Free base | 1 |
| | pyrazol-4-yl)-1,3-thiazole-4- | | | | |
| | carbonyl]-N-(propan-2- | | | | |
| | yl)piperidin-4-amine | | | | |
| 166 | 7-[2-(1-phenyl-1H-pyrazol-4-yl)- | 393 | 394 | Free base | 1 |
| | 1,3-thiazole-4-carbonyl]-1,7- | | | | |
| | diazaspiro[3.5]nonan-2-one | | | | |
| 168 | 2-methyl-4-[2-(1-phenyl-1H- | 354 | 355 | Free base | 1 |
| | pyrazol-4-yl)-1,3-thiazole-4- | | | | |
| | carbonyl]morpholine | | | | |
| 169 | N-methyl-N-[(oxolan-2- | 368 | 369 | Free base | 1 |
| | yl)methyl]-2-(1-phenyl-1H- | | | | |
| | pyrazol-4-yl)-1,3-thiazole-4- | | | | |
| | carboxamide | | | | |
| 172 | N-methyl-N-(oxan-4-yl)-2-(1- | 368 | 369 | Free base | 1 |
| | phenyl-1H-pyrazol-4-yl)-1,3- | | | | |
| | thiazole-4-carboxamide | | | | |
| 173 | N-ethyl-N-methyl-2-(1-phenyl- | 312 | 313 | Free base | 1 |
| | 1H-pyrazol-4-yl)-1,3-thiazole-4- | | | | |
| | carboxamide | | | | |
| 174 | N-(1-benzylpyrrolidin-3-yl)-2-(1- | 430 | 430 | Free base | 1 |
| | phenyl-1H-pyrazol-4-yl)-1,3- | | | | |
| | thiazole-4-carboxamide | | | | |
| 176 | N-[2-(4-hydroxyphenyl)ethyl]-2- | 390 | 391 | Free base | 1 |
| | (1-phenyl-1H-pyrazol-4-yl)-1,3- | | | | |
| | thiazole-4-carboxamide | | | | |
| 182 | N-(1-benzylpiperidin-4-yl)-2-(1- | 444 | 444 | Free base | 1 |
| | phenyl-1H-pyrazol-4-yl)-1,3- | | | | |
| | thiazole-4-carboxamide | | | | |
| 185 | N-(1-ethylpiperidin-4-yl)-2-(1- | 382 | 382 | Free base | 1 |
| | phenyl-1H-pyrazol-4-yl)-1,3- | | | | |
| | thiazole-4-carboxamide | | | | |
| 186 | N-{1-azabicyclo[2.2.2]octan-3- | 394 | 394 | Free base | 1 |
| | yl}-N-methyl-2-(1-phenyl-1H- | | | | |
| | pyrazol-4-yl)-1,3-thiazole-4- | | | | |
| | carboxamide | | | | |
| 187 | N,N-dimethyl-1-[2-(1-phenyl-1H- | 367 | 368 | Free base | 1 |
| | pyrazol-4-yl)-1,3-thiazole-4- | | | | |
| | carbonyl]pyrrolidin-3-amine | | | | |
| 188 | N-(1-ethylpyrrolidin-3-yl)-N- | 382 | 382 | Free base | 1 |
| | methyl-2-(1-phenyl-1H-pyrazol-4- | | | | |
| | yl)-1,3-thiazole-4-carboxamide | | | | |
| 189 | N-methyl-N-[(1-methylpyrrolidin- | 382 | 382 | Free base | 1 |
| | 3-yl)methyl]-2-(1-phenyl-1H- | | | | |
| | pyrazol-4-yl)-1,3-thiazole-4- | | | | |
| | carboxamide | | | | |
| 190 | N-{2- | 424 | 424 | Free base | 1 |
| | [cyclohexyl(methyl)amino]ethyl}- | | | | |
| | N-methyl-2-(1-phenyl-1H- | | | | |
| | pyrazol-4-yl)-1,3-thiazole-4- | | | | |
| | carboxamide | | | | |
| 191 | N-cyclopentyl-N-(2- | 395 | 395 | Free base | 1 |
| | methylpropyl)-2-(1-phenyl-1H- | | | | |
| | pyrazol-4-yl)-1,3-thiazole-4- | | | | |
| | carboxamide | | | | |
| 192 | N-cyclopentyl-N-ethyl-2-(1- | 366 | 367 | Free base | 1 |
| | phenyl-1H-pyrazol-4-yl)-1,3- | | | | |
| | thiazole-4-carboxamide | | | | |
| 193 | 1-ethyl-4-[2-(1-phenyl-1H- | 382 | 382 | Free base | 1 |
| | pyrazol-4-yl)-1,3-thiazole-4- | | | | |
| | carbonyl]-1,4-diazepane | | | | |
| 194 | N-cyclopentyl-N-cyclopropyl-2- | 378 | 379 | Free base | 1 |
| | (1-phenyl-1H-pyrazol-4-yl)-1,3- | | | | |
| | thiazole-4-carboxamide | | | | |
| 195 | N-methyl-2-(1-phenyl-1H- | 410 | 410 | Free base | 1 |
| | pyrazol-4-yl)-N-[1-(propan-2- | | | | |
| | yl)piperidin-4-yl]-1,3-thiazole-4- | | | | |
| | carboxamide | | | | |
| 196 | N-(1-ethylpiperidin-4-yl)-N- | 396 | 396 | Free base | see full |
| | methyl-2-(1-phenyl-1H-pyrazol-4- | | | | experimental |
| | yl)-1,3-thiazole-4-carboxamide | | | | |
| 197 | N,N-dimethyl-1-[2-(1-phenyl-1H- | 382 | 382 | Free base | 1 |
| | pyrazol-4-yl)-1,3-thiazole-4- | | | | |
| | carbonyl]piperidin-4-amine | | | | |
| 198 | 1-{4-[2-(1-phenyl-1H-pyrazol-4- | 381 | 382 | Free base | 1 |
| | yl)-1,3-thiazole-4- | | | | |
| | carbonyl]piperazin-1-yl}ethan-1- | | | | |
| | one | | | | |
| 199 | 1-ethyl-4-[2-(1-phenyl-1H- | 367 | 368 | Free base | 1 |
| | pyrazol-4-yl)-1,3-thiazole-4- | | | | |
| | carbonyl]piperazine | | | | |
| 200 | 4-[2-(1-phenyl-1H-pyrazol-4-yl)- | 353 | 354 | Free base | 1 |
| | 1,3-thiazole-4-carbonyl]piperazin- | | | | |
| | 2-one | | | | |
| 201 | N-cyclohexyl-N-methyl-2-(1- | 366 | 367 | Free base | 1 |
| | phenyl-1H-pyrazol-4-yl)-1,3- | | | | |
| | thiazole-4-carboxamide | | | | |
| 202 | 2-(1-phenyl-1H-pyrazol-4-yl)-N- | 382 | 382 | Free base | 1 |
| | [2-(piperidin-1-yl)ethyl]-1,3- | | | | |
| | thiazole-4-carboxamide | | | | |
| 203 | N-(cyclohexylmethyl)-2-(1- | 366 | 367 | Free base | 1 |
| | phenyl-1H-pyrazol-4-yl)-1,3- | | | | |
| | thiazole-4-carboxamide | | | | |
| 204 | N-[2-(morpholin-4-yl)ethyl]-2- | 383 | 384 | Free base | 1 |
| | (1-phenyl-1H-pyrazol-4-yl)-1,3- | | | | |
| | thiazole-4-carboxamide | | | | |
| 208 | N-[(3S)-1-methylpiperidin-3-yl]- | 367 | 368 | Free base | 1 |
| | 2-(1-phenyl-1H-pyrazol-4-yl)-1,3- | | | | |
| | thiazole-4-carboxamide | | | | |
| 209 | 1-[2-(1-phenyl-1H-pyrazol-4-yl)- | 382 | 382 | Free base | 1 |
| | 1,3-thiazole-4-carbonyl]-4- | | | | |
| | (propan-2-yl)piperazine | | | | |
| 210 | 2-{4-[2-(1-phenyl-1H-pyrazol-4- | 439 | 439 | Free base | 1 |
| | yl)-1,3-thiazole-4- | | | | |
| | carbonyl]piperazin-1-yl}-N- | | | | |
| | (propan-2-yl)acetamide | | | | |
| 211 | (3S)-1-{2-[1-(3-fluorophenyl)-1H- | 371.12 | 372 | HCl salt | 4 + 2 |
| | pyrazol-4-yl]-1,3-thiazole-4- | | | | |
| | carbonyl}-3-methylpiperazine | | | | |
| 212 | (3S)-1-{2-[1-(4-fluorophenyl)-1H- | 371.12 | 372.1 | HCl salt | 4 + 2 |
| | pyrazol-4-yl]-1,3-thiazole-4- | | | | |
| | carbonyl}-3-methylpiperazine | | | | |
| 213 | N-(1-ethylpiperidin-4-yl)-2-[1-(4- | 399.15 | 400.4 | Free base | 4 + 1 |
| | fluorophenyl)-1H-pyrazol-4-yl]-1,3- | | | | |
| | thiazole-4-carboxamide | | | | |
| 214 | N-(1-ethylpiperidin-4-yl)-5-(1-phenyl- | 363.21 | 364.1 | Free base | 3 + 1 |
| | 1H-pyrazol-4-yl)-1H-pyrrole-2- | | | | |
| | carboxamide | | | | |
| 215 | 2-(1-phenyl-1H-pyrazol-4-yl)-N-[1- | 395.18 | 396.3 | Free base | 1 |
| | (propan-2-yl)piperidin-4-yl]-1,3- | | | | |
| | thiazole-4-carboxamide | | | | |
| 216 | 4-(5-{1-[2-(trifluoromethyl)phenyl]- | 468.46 | 468.8 | HCl salt | 3 + 1 |
| | 1H-pyrazol-4-yl}-1H-pyrrole-2- | | | | |
| | carbonyl)piperazine-1-sulfonamide | | | | |
| 217 | N-(1-ethylpiperidin-4-yl)-N-methyl-2- | 463.17 | 464.1 | Free base | 4 + 1 |
| | {1-[2-(trifluoromethyl)phenyl]-1H- | | | | |
| | pyrazol-4-yl}-1,3-thiazole-4- | | | | |
| | carboxamide | | | | |
| 218 | N-(1-ethylpiperidin-4-yl)-2-(1-phenyl- | 423.58 | 424.1 | Free base | 2 (with |
| | 1H-pyrazol-4-yl)-N-(propan-2-yl)-1,3- | | | | CAS No. |
| | thiazole-4-carboxamide | | | | 534595-51-2) |
| | | | | | then |
| | | | | | alkylation of |
| | | | | | piperidine |
| | | | | | NH |
| 219 | N-ethyl-N-(1-ethylpiperidin-4-yl)-2- | 409.55 | 410.19 | Free base | 1 |
| | (1-phenyl-1H-pyrazol-4-yl)-1,3- | | | | |
| | thiazole-4-carboxamide | | | | |
| 220 | (3S)-3-methyl-1-{2-[1-(pyridin-2-yl)- | 353.14 | 354.9 | HCl salt | 4 + 2 |
| | 1H-pyrazol-4-yl]-1,3-thiazole-4- | | | | |
| | carbonyl}piperazine | | | | |
| 221 | N-(1-ethylpiperidin-4-yl)-N-methyl-2- | 396.51 | 397 | Free base | 4 + 1 |
| | [1-(pyridin-2-yl)-1H-pyrazol-4-yl]- | | | | |
| | 1,3-thiazole-4-carboxamide | | | | |
| 222 | N-(1-ethylpiperidin-4-yl)-2-[1- | 382.16 | 383 | Free base | 4 + 1 |
| | (pyridin-2-yl)-1H-pyrazol-4-yl]-1,3- | | | | |
| | thiazole-4-carboxamide | | | | |
| 223 | (3S)-3-methyl-1-{2-[1-(pyridin-3-yl)- | 354.43 | 354.9 | Free base | 4 + 2 |
| | 1H-pyrazol-4-yl]-1,3-thiazole-4- | | | | |
| | carbonyl}piperazine | | | | |
| 224 | N-(1-ethylpiperidin-4-yl)-N-methyl-2- | 396.17 | 397 | Free base | 4 + 1 |
| | [1-(pyridin-3-yl)-1H-pyrazol-4-yl]- | | | | |
| | 1,3-thiazole-4-carboxamide | | | | |
| 225 | N-(1-ethylpiperidin-4-yl)-2-[1- | 382.49 | 382.9 | Free base | 4 + 1 |
| | (pyridin-3-yl)-1H-pyrazol-4-yl]-1,3- | | | | |
| | thiazole-4-carboxamide | | | | |
| 226 | (3S)-3-methyl-1-{2-[1-(pyridin-4-yl)- | 354.43 | 354.9 | HCl salt | 4 + 2 |
| | 1H-pyrazol-4-yl]-1,3-thiazole-4- | | | | |
| | carbonyl}piperazine | | | | |
| 227 | N-(1-ethylpiperidin-4-yl)-N-methyl-2- | 396.51 | 397 | Free base | 4 + 1 |
| | [1-(pyridin-4-yl)-1H-pyrazol-4-yl]- | | | | |
| | 1,3-thiazole-4-carboxamide | | | | |
| 228 | N-(1-ethylpiperidin-4-yl)-2-[1- | 382.49 | 383 | Free base | 4 + 1 |
| | (pyridin-4-yl)-1H-pyrazol-4-yl]-1,3- | | | | |
| | thiazole-4-carboxamide | | | | |
| 229 | 3-(4-{4-[(3S)-3-methylpiperazine-1- | 355.42 | 356 | HCl salt | 4 + 2 |
| | carbonyl]-1,3-thiazol-2-yl}-1H- | | | | |
| | pyrazol-1-yl)pyridazine | | | | |
| 230 | N-(1-ethylpiperidin-4-yl)-N-methyl-2- | 397.5 | 398 | Free base | 4 + 1 |
| | [1-(pyridazin-3-yl)-1H-pyrazol-4-yl]- | | | | |
| | 1,3-thiazole-4-carboxamide | | | | |
| 231 | N-(1-ethylpiperidin-4-yl)-2-[1- | 383.47 | 383.9 | Free base | 4 + 1 |
| | (pyridazin-3-yl)-1H-pyrazol-4-yl]-1,3- | | | | |
| | thiazole-4-carboxamide | | | | |
| 232 | 2-(4-{4-[(3S)-3-methylpiperazine-1- | 355.42 | 355.9 | HCl salt | 4 + 2 |
| | carbonyl]-1,3-thiazol-2-yl}-1H- | | | | |
| | pyrazol-1-yl)pyrazine | | | | |
| 233 | N-(1-ethylpiperidin-4-yl)-N-methyl-2- | 397.5 | 398.1 | Free base | 4 + 1 |
| | [1-(pyrazin-2-yl)-1H-pyrazol-4-yl]- | | | | |
| | 1,3-thiazole-4-carboxamide | | | | |
| 234 | N-(1-ethylpiperidin-4-yl)-2-[1- | 383.47 | 384 | Free base | 4 + 1 |
| | (pyrazin-2-yl)-1H-pyrazol-4-yl]-1,3- | | | | |
| | thiazole-4-carboxamide | | | | |
| 235 | 3,3-dimethyl-1-[2-(1-phenyl-1H- | 367.47 | 367.9 | HCl salt | 2 |
| | pyrazol-4-yl)-1,3-thiazole-4- | | | | |
| | carbonyl]piperazine | | | | |
| 236 | 1-ethyl-4-[2-(1-phenyl-1H-pyrazol-4- | 367.15 | 367.9 | HCl salt | 1 |
| | yl)-1,3-thiazole-4-carbonyl]piperazine | | | | |
| 237 | (3R,5S)-3,5-dimethyl-1-[2-(1-phenyl- | 367.15 | 368.2 | HCl salt | 2 |
| | 1H-pyrazol-4-yl)-1,3-thiazole-4- | | | | |
| | carbonyl]piperazine | | | | |
| 238 | (3S)-3-methyl-1-[2-(2-phenyl-2H- | 354.43 | 355 | HCl salt | 2 (using |
| | 1,2,3-triazol-4-yl)-1,3-thiazole-4- | | | | CAS No. |
| | carbonyl]piperazine | | | | 1094456-13-9) |
| 239 | N-(1-tert-butylpiperidin-4-yl)-N- | 423.58 | 424.3 | Free base | 1 |
| | methyl-2-(1-phenyl-1H-pyrazol-4-yl)- | | | | |
| | 1,3-thiazole-4-carboxamide | | | | |
| 240 | N-methyl-2-(1-phenyl-1H-pyrazol-4- | 409.55 | 410.1 | Free base | 1 |
| | yl)-N-[1-(propan-2-yl)piperidin-4-yl]- | | | | |
| | 1,3-thiazole-4-carboxamide | | | | |
| 241 | N-methyl-N-[(2S)-2-methylpiperidin- | 381.16 | 382.1 | HCl Salt | 2 |
| | 4-yl]-2-(1-phenyl-1H-pyrazol-4-yl)- | | | | |
| | 1,3-thiazole-4-carboxamide | | | | |
| 242 | N-[(2S)-1-ethyl-2-methylpiperidin-4- | 409.55 | 410 | Free base | from |
| | yl]-N-methyl-2-(1-phenyl-1H-pyrazol- | | | | Compound |
| | 4-yl)-1,3-thiazole-4-carboxamide | | | | 41 |
| | | | | | (alkylation of |
| | | | | | piperidine |
| | | | | | NH with EtI) |
| 243 | N-[(2S)-1-ethyl-2-methylpiperidin-4- | 395.53 | 396.1 | Free base | 2 then |
| | yl]-2-(1-phenyl-1H-pyrazol-4-yl)-1,3- | | | | alkylation |
| | thiazole-4-carboxamide | | | | of piperidine |
| | | | | | NH |
| | | | | | with EtI |
| 244 | N-[(1R,4R)-2-ethyl-2- | 393.51 | 394.3 | Free base | 2 then |
| | azabicyclo[2.2.1]heptan-5-yl]-2-(1- | | | | alkylation |
| | phenyl-1H-pyrazol-4-yl)-1,3-thiazole- | | | | of piperidine |
| | 4-carboxamide | | | | NH |
| | | | | | with EtI |
| 245 | N-[(1S,4S)-2-ethyl-2- | 407.54 | 408.2 | HCl salt | 2 then |
| | azabicyclo[2.2.1]heptan-5-yl]-N- | | | | alkylation |
| | methyl-2-(1-phenyl-1H-pyrazol-4-yl)- | | | | of piperidine |
| | 1,3-thiazole-4-carboxamide | | | | NH |
| | | | | | with EtI |
| 246 | N-[(1S,4S)-2-ethyl-2- | 393.51 | 394.3 | Free base | 2 then |
| | azabicyclo[2.2.1]heptan-5-yl]-2-(1- | | | | alkylation |
| | phenyl-1H-pyrazol-4-yl)-1,3-thiazole- | | | | of piperidine |
| | 4-carboxamide | | | | NH |
| | | | | | with EtBr |
| 247 | N-(1-ethyl-1H-pyrrol-3-yl)-N-methyl- | 377.47 | 378 | Free base | 2 then |
| | 2-(1-phenyl-1H-pyrazol-4-yl)-1,3- | | | | alkylation |
| | thiazole-4-carboxamide | | | | of amide-NH |
| | | | | | with MeI |
| 248 | N-(1-tert-butylpiperidin-4-yl)-2-(1- | 409.55 | 410 | Free base | 1 |
| | phenyl-1H-pyrazol-4-yl)-1,3-thiazole- | | | | |
| | 4-carboxamide | | | | |
| 249 | 2-(4-{4-[(3S)-3-methylpiperazine-1- | 355.42 | 355.9 | HCl salt | 4 + 2 |
| | carbonyl]-1,3-thiazol-2-yl}-1H- | | | | |
| | pyrazol-1-yl)pyrimidine | | | | |
| 250 | N-(1-ethylpiperidin-4-yl)-N-methyl-2- | 397.5 | 398 | Free base | 4 + 1 |
| | [1-(pyrimidin-2-yl)-1H-pyrazol-4-yl]- | | | | |
| | 1,3-thiazole-4-carboxamide | | | | |
| 251 | N-(1-ethylpiperidin-4-yl)-2-[1- | 383.47 | 383.9 | Free base | 4 + 1 |
| | (pyrimidin-2-yl)-1H-pyrazol-4-yl]-1,3- | | | | |
| | thiazole-4-carboxamide | | | | |
| 252 | 5-(4-{4-[(3S)-3-methylpiperazine-1- | 355.42 | 356 | HCl Salt | 4 + 2 |
| | carbonyl]-1,3-thiazol-2-yl}-1H- | | | | |
| | pyrazol-1-yl)pyrimidine | | | | |
| 253 | N-(1-ethylpiperidin-4-yl)-N-methyl-2- | 397.5 | 398 | HCl Salt | 4 + 1 |
| | [1-(pyrimidin-5-yl)-1H-pyrazol-4-yl]- | | | | |
| | 1,3-thiazole-4-carboxamide | | | | |
| 254 | N-(1-ethylpiperidin-4-yl)-2-[1- | 383.47 | 384 | HCl Salt | 4 + 1 |
| | (pyrimidin-5-yl)-1H-pyrazol-4-yl]-1,3- | | | | |
| | thiazole-4-carboxamide | | | | |
| 255 | (3S)-3-methyl-1-[4-(1-phenyl-1H- | 335.41 | 335.9 | HCl salt | 3 + 2 |
| | pyrazol-4-yl)-1H-pyrrole-2- | | | | |
| | carbonyl]piperazine | | | | |
| 256 | N-(1-ethylpiperidin-4-yl)-N-methyl-4- | 377.49 | 378.2 | Free base | 3 + 1 |
| | (1-phenyl-1H-pyrazol-4-yl)-1H- | | | | |
| | pyrrole-2-carboxamide | | | | |
| 257 | N-(1-ethylpiperidin-4-yl)-4-(1-phenyl- | 363.47 | 364.2 | Free base | 3 + 1 |
| | 1H-pyrazol-4-yl)-1H-pyrrole-2- | | | | |
| | carboxamide | | | | |
| 258 | N-(1-ethylpiperidin-4-yl)-N-methyl-4- | 412.57 | 413 | Free base | 3 + 1 |
| | (5-phenyl-1,3,4-thiadiazol-2- | | | | |
| | yl)thiophene-2-carboxamide | | | | |
| 259 | N-(1-ethylpiperidin-4-yl)-4-(5-phenyl- | 398.54 | 399 | Free base | 3 + 1 |
| | 1,3,4-thiadiazol-2-yl)thiophene-2- | | | | |
| | carboxamide | | | | |
| 260 | (3S)-3-methyl-1-[2-(1-phenyl-1H- | 354.43 | 355.1 | HCl salt | 3 + 2 |
| | 1,2,4-triazol-3-yl)-1,3-thiazole-4- | | | | |
| | carbonyl]piperazine | | | | |
| 261 | N-(1-ethylpiperidin-4-yl)-N-methyl-2- | 396.51 | 397.2 | Free base | 3 + 1 |
| | (1-phenyl-1H-1,2,4-triazol-3-yl)-1,3- | | | | |
| | thiazole-4-carboxamide | | | | |
| 262 | N-(1-ethylpiperidin-4-yl)-2-(1-phenyl- | 382.49 | 383.1 | Free base | 3 + 1 |
| | 1H-1,2,4-triazol-3-yl)-1,3-thiazole-4- | | | | |
| | carboxamide | | | | |
| 263 | (3S)-3-methyl-1-[2-(1-phenyl-1H- | 337.38 | 338.1 | HCl salt | 3 + 2 |
| | pyrazol-4-yl)-1,3-oxazole-4- | | | | |
| | carbonyl]piperazine | | | | |
| 264 | N-(1-ethylpiperidin-4-yl)-N-methyl-2- | 379.46 | 380.2 | Free base | 3 + 1 |
| | (1-phenyl-1H-pyrazol-4-yl)-1,3- | | | | |
| | oxazole-4-carboxamide | | | | |
| 265 | N-(1-ethylpiperidin-4-yl)-2-(1-phenyl- | 365.44 | 366.2 | Free base | 3 + 1 |
| | 1H-pyrazol-4-yl)-1,3-oxazole-4- | | | | |
| | carboxamide | | | | |
| 266 | (3S)-1-{2-[1-(4-cyclopropylphenyl)- | 393.51 | 394.1 | HCl salt | 4 + 5A |
| | 1H-pyrazol-4-yl]-1,3-thiazole-4- | | | | |
| | carbonyl}-3-methylpiperazine | | | | |
| 267 | (3S)-1-{2-[1-(4-cyclopropylphenyl)- | 393.51 | 394.1 | HCl salt | 4 + 5A |
| | 1H-pyrazol-4-yl]-1,3-thiazole-4- | | | | |
| | carbonyl}-3-methylpiperazine | | | | |
| 268 | 2-[1-(4-cyclopropylphenyl)-1H- | 421.56 | 422.1 | Free base | 4 + 5B |
| | pyrazol-4-yl]-N-(1-ethylpiperidin-4- | | | | |
| | yl)-1,3-thiazole-4-carboxamide | | | | |
| 269 | 2-[1-(4-cyclopropylphenyl)-1H- | 435.59 | 436 | Free base | 4 + 5B |
| | pyrazol-4-yl]-N-(1-ethylpiperidin-4- | | | | |
| | yl)-N-methyl-1,3-thiazole-4- | | | | |
| | carboxamide | | | | |
| 270 | (3S)-1-{2-[1-(4-tert-butylphenyl)-1H- | 409.55 | 410.1 | HCl salt | 4 + 5A |
| | pyrazol-4-yl]-1,3-thiazole-4- | | | | |
| | carbonyl}-3-methylpiperazine | | | | |
| 271 | 2-[1-(4-tert-butylphenyl)-1H-pyrazol- | 437.61 | 438.1 | Free base | 4 + 5B |
| | 4-yl]-N-(1-ethylpiperidin-4-yl)-1,3- | | | | |
| | thiazole-4-carboxamide | | | | |
| 272 | 2-[1-(4-tert-butylphenyl)-1H-pyrazol- | 451.63 | 452.2 | Free base | 4 + 5B |
| | 4-yl]-N-(1-ethylpiperidin-4-yl)-N- | | | | |
| | methyl-1,3-thiazole-4-carboxamide | | | | |
| 273 | (3S)-3-methyl-1-(2-{1-[4-(propan-2- | 395.53 | 396.1 | HCl salt | 4 + 5A |
| | yl)phenyl]-1H-pyrazol-4-yl}-1,3- | | | | |
| | thiazole-4-carbonyl)piperazine | | | | |
| 274 | N-(1-ethylpiperidin-4-yl)-2-{1-[4- | 423.58 | 424.1 | Free base | 4 + 5B |
| | (propan-2-yl)phenyl]-1H-pyrazol-4- | | | | |
| | yl}-1,3-thiazole-4-carboxamide | | | | |
| 275 | N-(1-ethylpiperidin-4-yl)-N-methyl-2- | 437.61 | 438 | Free base | 4 + 5B |
| | {1-[4-(propan-2-yl)phenyl]-1H- | | | | |
| | pyrazol-4-yl}-1,3-thiazole-4- | | | | |
| | carboxamide | | | | |
| 276 | (3S)-3-methyl-1-[2-(1-phenyl-1H- | 336.4 | 336.9 | HCl salt | 3 + 2 |
| | pyrazol-4-yl)-1H-imidazole-4- | | | | |
| | carbonyl]piperazine | | | | |
| 277 | N-(1-ethylpiperidin-4-yl)-N-methyl-2- | 378.48 | 379 | Free base | 3 + 1 |
| | (1-phenyl-1H-pyrazol-4-yl)-1H- | | | | |
| | imidazole-4-carboxamide | | | | |
| 278 | N-(1-ethylpiperidin-4-yl)-2-(1-phenyl- | 364.45 | 365 | Free base | 3 + 1 |
| | 1H-pyrazol-4-yl)-1H-imidazole-4- | | | | |
| | carboxamide | | | | |
| 279 | (3S)-3-methyl-1-[5-(1-phenyl-1H- | 336.4 | 337 | HCl salt | 3 + 2 |
| | pyrazol-4-yl)-1H-imidazole-2- | | | | |
| | carbonyl]piperazine | | | | |
| 280 | N-(1-ethylpiperidin-4-yl)-N-methyl-5- | 378.48 | 379 | Free base | 3 + 1 |
| | (1-phenyl-1H-pyrazol-4-yl)-1H- | | | | |
| | imidazole-2-carboxamide | | | | |
| 281 | N-(1-ethylpiperidin-4-yl)-5-(1-phenyl- | 364.45 | 365.1 | Free base | 3 + 1 |
| | 1H-pyrazol-4-yl)-1H-imidazole-2- | | | | |
| | carboxamide | | | | |
| 282 | N-(1-ethylpiperidin-4-yl)-N-(propan- | 424.57 | 425.3 | Free base | 4 + 1 |
| | 2-yl)-2-[1-(pyridin-2-yl)-1H-pyrazol- | | | | |
| | 4-yl]-1,3-thiazole-4-carboxamide | | | | |
| 283 | N-ethyl-N-(1-ethylpiperidin-4-yl)-2- | 410.54 | 411.3 | Free base | 4 + 1 |
| | [1-(pyridin-2-yl)-1H-pyrazol-4-yl]- | | | | |
| | 1,3-thiazole-4-carboxamide | | | | |
| 284 | N-(1-ethylpiperidin-4-yl)-N-methyl-2- | 379.47 | 380 | Free base | 3 + 2 |
| | [1-(pyridin-2-yl)-1H-pyrazol-4-yl]- | | | | |
| | 1H-imidazole-4-carboxamide | | | | |
| 285 | N-ethyl-N-(1-ethylpiperidin-4-yl)-2- | 393.5 | 394 | Free base | 3 + 1 |
| | [1-(pyridin-2-yl)-1H-pyrazol-4-yl]- | | | | |
| | 1H-imidazole-4-carboxamide | | | | |
| 286 | N-(1-ethylpiperidin-4-yl)-N-methyl-2- | 380.2 | 381.1 | Free base | 3 + 2 |
| | [1-(pyridin-2-yl)-1H-pyrazol-4-yl]- | | | | |
| | 1,3-oxazole-4-carboxamide | | | | |
| 287 | N-ethyl-N-(1-ethylpiperidin-4-yl)-2- | 394.48 | 395.1 | Free base | 3 + 1 |
| | [1-(pyridin-2-yl)-1H-pyrazol-4-yl]- | | | | |
| | 1,3-oxazole-4-carboxamide | | | | |
| 288 | N-(1-ethylpiperidin-4-yl)-N-methyl-2- | 479.52 | 480.1 | Free base | 4 + 1 |
| | {1-[2-(trifluoromethoxy)phenyl]-1H- | | | | |
| | pyrazol-4-yl}-1,3-thiazole-4- | | | | |
| | carboxamide | | | | |
| 289 | (3S)-3-methyl-1-(2-{1-[2- | 437.44 | 438 | HCl salt | 4 + 2 |
| | (trifluoromethoxy)phenyl]-1H- | | | | |
| | pyrazol-4-yl}-1,3-thiazole-4- | | | | |
| | carbonyl)piperazine | | | | |
| 290 | N-(1-ethylpiperidin-4-yl)-2-{1-[2- | 465.5 | 466.3 | Free base | 4 + 1 |
| | (trifluoromethoxy)phenyl]-1H- | | | | |
| | pyrazol-4-yl}-1,3-thiazole-4- | | | | |
| | carboxamide | | | | |
| 291 | N-(1-ethylpiperidin-4-yl)-N-methyl-2- | 463.46 | 464.1 | Free base | 3 + 1 |
| | {1-[2-(trifluoromethoxy)phenyl]-1H- | | | | |
| | pyrazol-4-yl}-1,3-oxazole-4- | | | | |
| | carboxamide | | | | |
| 292 | (3S)-3-methyl-1-(2-{1-[2- | 421.38 | 422.1 | HCl salt | 3 + 2 |
| | (trifluoromethoxy)phenyl]-1H- | | | | |
| | pyrazol-4-yl}-1,3-oxazole-4- | | | | |
| | carbonyl)piperazine | | | | |
| 293 | N-(1-ethylpiperidin-4-yl)-2-{1-[2- | 449.43 | 450.1 | Free base | 3 + 1 |
| | (trifluoromethoxy)phenyl]-1H- | | | | |
| | pyrazol-4-yl}-1,3-oxazole-4- | | | | |
| | carboxamide | | | | |
| 294 | N-(1-methylpiperidin-4-yl)-2-(1- | 409.55 | 410 | Free base | 2 (with |
| | phenyl-1H-pyrazol-4-yl)-N-(propan-2- | | | | CAS No. |
| | yl)-1,3-thiazole-4-carboxamide | | | | 534595-51-2) |
| | | | | | then |
| | | | | | alkylation of |
| | | | | | piperidine |
| | | | | | NH |
| | | | | | with MeI |
| 295 | 2-(1-phenyl-1H-pyrazol-4-yl)-N- | 437.61 | 438 | Free base | 2 (with |
| | (propan-2-yl)-N-[1-(propan-2- | | | | CAS No. |
| | yl)piperidin-4-yl]-1,3-thiazole-4- | | | | 534595-51-2) |
| | carboxamide | | | | then |
| | | | | | alkylation of |
| | | | | | piperidine |
| | | | | | NH with |
| | | | | | isopropyl- |
| | | | | | bromide |
| 296 | N-(1-tert-butylpiperidin-4-yl)-2-(1- | 451.63 | 452 | Free base | 1 |
| | phenyl-1H-pyrazol-4-yl)-N-(propan-2- | | | | |
| | yl)-1,3-thiazole-4-carboxamide | | | | |
| 297 | N-(2-methylpiperidin-4-yl)-2-(1- | 409.55 | 410. | HCl salt | 2 |
| | phenyl-1H-pyrazol-4-yl)-N-(propan-2- | | 820 | | |
| | yl)-1,3-thiazole-4-carboxamide | | (M + 1 × | | |
| | | | 2) | | |
| 298 | N-(2,6-dimethylpiperidin-4-yl)-2-(1- | 423.58 | 424.1 | HCl salt | 2 |
| | phenyl-1H-pyrazol-4-yl)-N-(propan-2- | | | | |
| | yl)-1,3-thiazole-4-carboxamide | | | | |
| 299 | N-[(3S,5R)-5-methylpyrrolidin-3-yl]- | 395.53 | 396.1 | HCl salt | 2 |
| | 2-(1-phenyl-1H-pyrazol-4-yl)-N- | | | | |
| | (propan-2-yl)-1,3-thiazole-4- | | | | |
| | carboxamide | | | | |
| 300 | N-[(3R)-1-(3- | 449.56 | 450.1 | HCl salt | 3 + 2, then |
| | methoxypropanoyl)pyrrolidin-3-yl]-5- | | | | acylation |
| | (1-phenyl-1H-pyrazol-4-yl)-N-propyl- | | | | of amine |
| | 1H-pyrrole-2-carboxamide | | | | with CAS |
| | | | | | No. 4244-59-1 |
| 301 | (3S)-3-methyl-1-[2-(1-methyl-1H- | 291.37 | 291.9 | HCl salt | 4 + 2 |
| | pyrazol-4-yl)-1,3-thiazole-4- | | | | |
| | carbonyl]piperazine | | | | |
| 302 | (3S)-3-methyl-1-{2-[1-(propan-2-yl)- | 319.43 | 319.9 | HCl salt | See |
| | 1H-pyrazol-4-yl]-1,3-thiazole-4- | | | | Experimental |
| | carbonyl}piperazine | | | | details |
| 303 | (3S)-1-[2-(1-tert-butyl-1H-pyrazol-4- | 333.45 | 333.9 | HCl salt | 4 + 2 |
| | yl)-1,3-thiazole-4-carbonyl]-3- | | | | |
| | methylpiperazine | | | | |
| 304 | N-[(3R)-1-(2- | 421.55 | 422.2 | Free base | 3 + 2, then |
| | methoxyethyl)pyrrolidin-3-yl]-5-(1- | | | | acylation |
| | phenyl-1H-pyrazol-4-yl)-N-propyl- | | | | of alkylation |
| | 1H-pyrrole-2-carboxamide | | | | with CAS |
| | | | | | No. 6482-24-2 |

TABLE 2
Compounds according to the present invention, chemical structures, and names

| Compound/ | | |
|---|---|---|
| Formula | | |
| Number | Structure | Chemical Name |
| 1 | 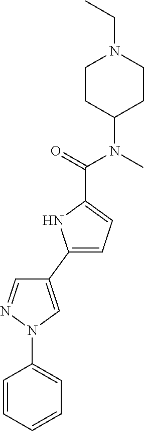 [SMILES: CCN1CCC(N(C)C(=O)c2ccc(-c3cnn(-c4ccccc4)c3)[nH]2)CC1] | N-(1-ethylpiperidin-4-yl)-N- methyl-5-(1-phenyl-1H- pyrazol-4-yl)-1H-pyrrole-2- carboxamide |
| 2 | 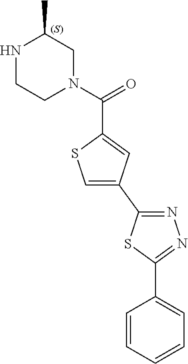 | (3S)-3-methyl-1-[4-(5- phenyl-1,3,4-thiadiazol-2- yl)thiophene-2- carbonyl]piperazine |
| 3 | 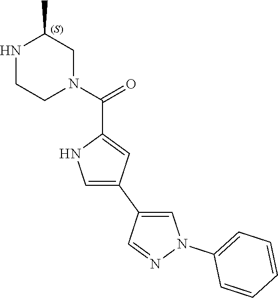 | (3S)-3-methyl-1-[4-(1- phenyl-1H-pyrazol-4-yl)- 1H-pyrrole-2- carbonyl]piperazine |
| 4 | 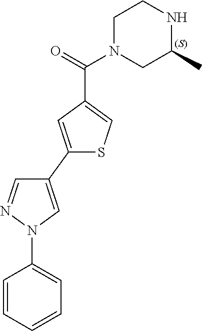 | (3S)-3-methyl-1-[5-(1- phenyl-1H-pyrazol-4- yl)thiophene-3- carbonyl]piperazine |
| 7 | 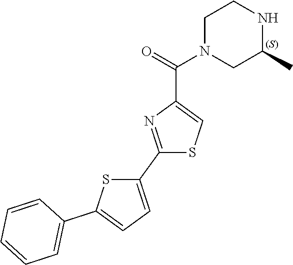 | (3S)-3-methyl-1-[2-(5- phenylthiophen-2-yl)-1,3- thiazole-4- carbonyl]piperazine |
| 10 | 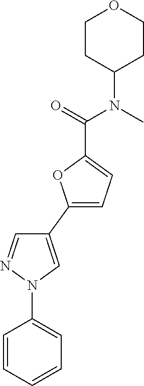 [SMILES: CN(C(=O)c1ccc(-c2cnn(-c3ccccc3)c2)o1)C1CCOCC1] | N-methyl-N-(oxan-4-yl)-5- (1-phenyl-1H-pyrazol-4- yl)furan-2-carboxamide |
| 13 | 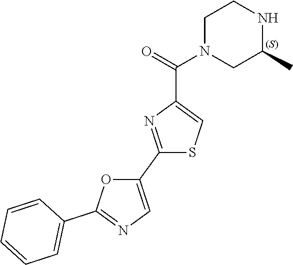 | (3S)-3-methyl-1-[2-(2- phenyl-1,3-oxazol-5-yl)-1,3- thiazole-4- carbonyl]piperazine |
| 15 | 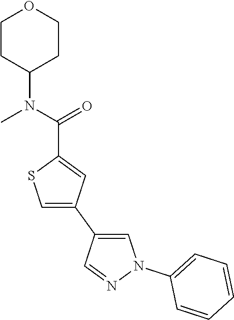 [SMILES: CN(C(=O)c1cc(-c2cnn(-c3ccccc3)c2)cs1)C1CCOCC1] | N-methyl-N-(oxan-4-yl)-4- (1-phenyl-1H-pyrazol-4- yl)thiophene-2-carboxamide |
| 19 | 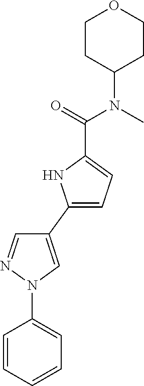 [SMILES: CN(C(=O)c1ccc(-c2cnn(-c3ccccc3)c2)[nH]1)C1CCOCC1] | N-methyl-N-(oxan-4-yl)-5-(1- phenyl-1H-pyrazol-4-yl)-1H- pyrrole-2-carboxamide |
| 24 | 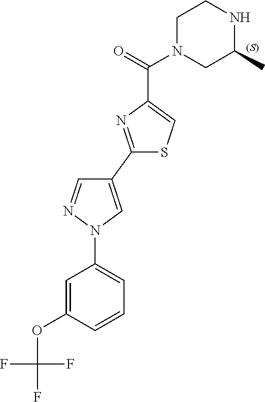 | (3S)-3-methyl-1-(2-{1-[3- (trifluoromethoxy)phenyl]- 1H-pyrazol-4-yl}-1,3- thiazole-4- carbonyl)piperazine |
| 27 | 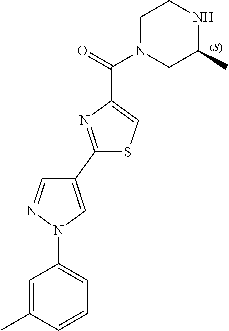 | (3S)-3-methyl-1-{2-[1-(3- methylphenyl)-1H-pyrazol- 4-yl]-1,3-thiazole-4- carbonyl}piperazine |
| 30 | 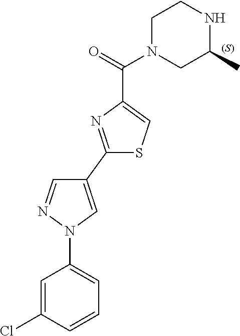 | (3S)-1-{2-[1-(3- chlorophenyl)-1H-pyrazol-4- yl]-1,3-thiazole-4- carbonyl}-3- methylpiperazine |
| 33 | 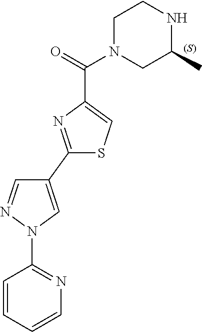 | (3S)-3-methyl-1-{2-[1- (pyridin-2-yl)-1H-pyrazol-4- yl]-1,3-thiazole-4- carbonyl}piperazine |
| 34 | 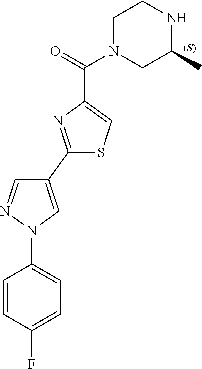 | (3S)-1-{2-[1-(4- fluorophenyl)-1H-pyrazol-4 yl]-1,3-thiazole-4-carbonyl}- 3-methylpiperazine |
| 37 | 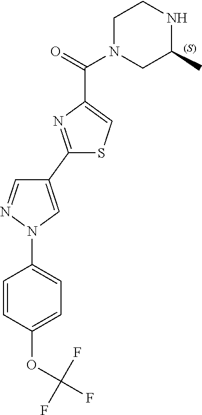 | (3S)-3-methyl-1-(2-{1-[4- (trifluoromethoxy)phenyl]- 1H-pyrazol-4-yl}-1,3- thiazole-4- carbonyl)piperazine |
| 38 | 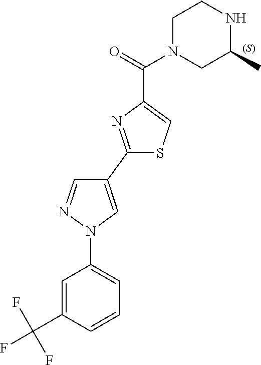 | (3S)-3-methyl-1-(2-{1-[3- (trifluoromethyl)phenyl]- 1H-pyrazol-4-yl}-1,3- thiazole-4- carbonyl)piperazine |
| 39 | 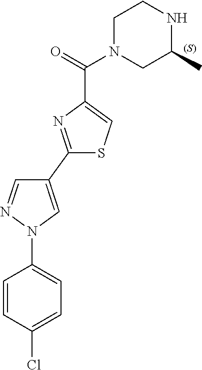 | (3S)-1-{2-[1-(4- chlorophenyl)-1H-pyrazol-4- yl]-1,3-thiazole-4- carbonyl}-3- methylpiperazine |
| 55 | 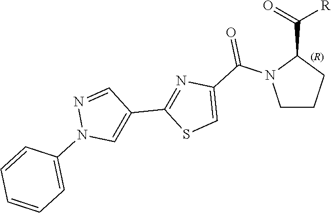 | (2R)-1-[2-(1-phenyl-1H- pyrazol-4-yl)-1,3-thiazole-4 carbonyl]pyrrolidine-2- carboxylic acid |
| 60 | 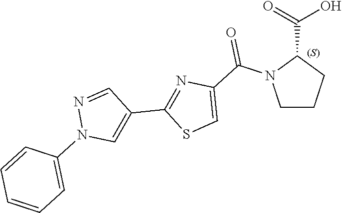 | (2S)-1-[2-(1-phenyl-1H- pyrazol-4-yl)-1,3-thiazole-4 carbonyl]pyrrolidine-2- carboxylic acid |
| 62 | 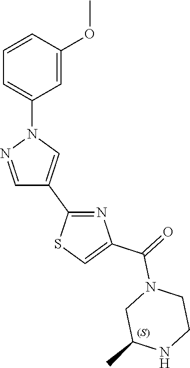 | (3S)-1-{2-[1-(3- methoxyphenyl)-1H-pyrazol- 4-yl]-1,3-thiazole-4 carbonyl}-3- methylpiperazine |
| 63 | 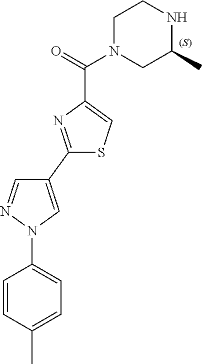 | (3S)-3-methyl-1-{2-[1-(4- methylphenyl)-1H-pyrazol- 4-yl]-1,3-thiazole-4- carbonyl}piperazine |
| 67 | 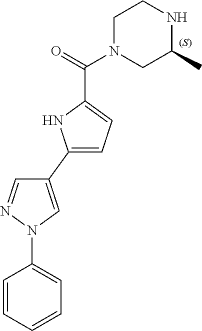 | (3S)-3-methyl-1-[5-(1- phenyl-1H-pyrazol-4-yl)- 1H-pyrrole-2- carbonyl]piperazine |
| 72 | 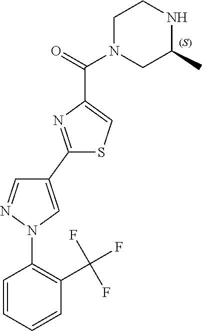 | (3S)-3-methyl-1-(2-{1-[2- (trifluoromethyl)phenyl]- 1H-pyrazol-4-yl}-1,3- thiazole-4- carbonyl)piperazine |
| 75 | 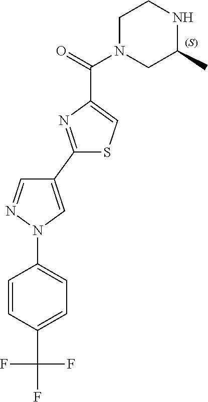 | (3S)-3-methyl-1-(2-{1-[4- (trifluoromethyl)phenyl]- 1H-pyrazol-4-yl}-1,3- thiazole-4- carbonyl)piperazine |
| 79 | 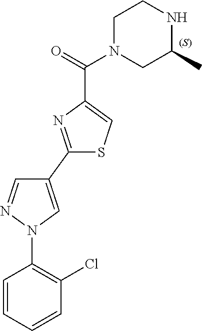 | (3S)-1-{2-[1-(2- chlorophenyl)-1H-pyrazol-4-yl]-1,3- thiazole-4-carbonyl}-3- methylpiperazine |
| 85 | 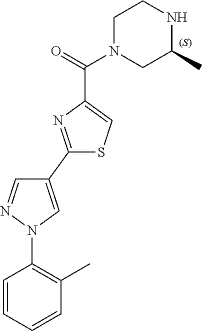 | (3S)-3-methyl-1-{2-[1-(2- methylphenyl)-1H-pyrazol-4-yl]-1,3- thiazole-4-carbonyl}piperazine |
| 96 | 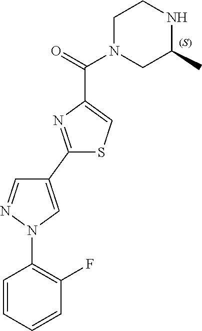 | (3S)-1-{2-[1-(2- fluorophenyl)-1H-pyrazol-4 yl]-1,3- thiazole-4-carbonyl}-3-methylpiperazine |
| 102 | 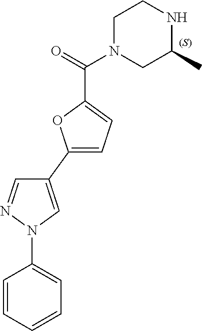 | (3S)-3-methyl-1-[5-(1- phenyl-1H-pyrazol-4-yl)furan-2- carbonyl]piperazine |
| 103 | 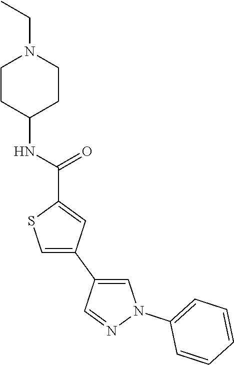 [SMILES: CCN1CCC(NC(=O)c2cc(-c3cnn(-c4ccccc4)c3)cs2)CC1] | N-(1-ethylpiperidin-4-yl)-4-(1-phenyl- 1H-pyrazol-4-yl)thiophene-2- carboxamide |
| 105 | 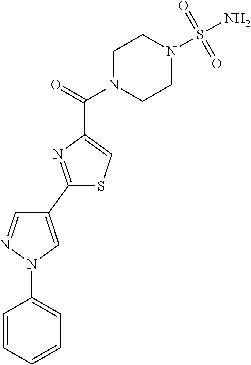 [SMILES: NS(=O)(=O)N1CCN(C(=O)c2csc(-c3cnn(-c4ccccc4)c3)n2)CC1] | 4-[2-(1-phenyl-1H-pyrazol- 4-yl)-1,3-thiazole-4- carbonyl]piperazine-1-sulfonamide |
| 109 | 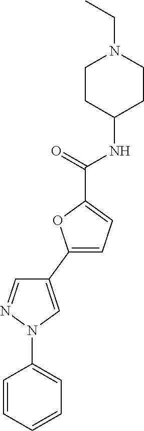 [SMILES: CCN1CCC(NC(=O)c2ccc(-c3cnn(-c4ccccc4)c3)o2)CC1] | N-(1-ethylpiperidin-4-yl)-5-(1-phenyl- 1H-pyrazol-4-yl)furan-2-carboxamide |
| 113 | 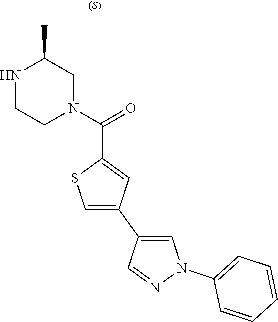 [SMILES: "(S)" ; C[C@H]1CN(C(=O)c2cc(-c3cnn(-c4ccccc4)c3)cs2)CCN1] | (3S)-3-methyl-1-[4-(1- phenyl-1H-pyrazol-4-yl)thiophene- 2-carbonyl]piperazine |
| 118 | 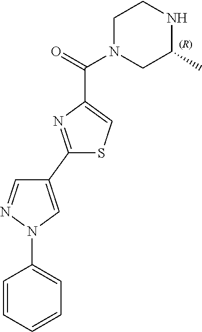 | (3R)-3-methyl-1-[2-(1- phenyl-1H-pyrazol-4-yl)-1,3-thiazole- 4-carbonyl]piperazine |
| 125 | 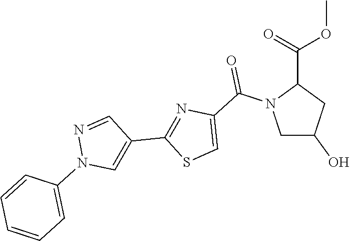 [SMILES: COC(=O)C1CC(O)CN1C(=O)c1csc(-c2cnn(-c3ccccc3)c2)n1] | methyl 4-hydroxy-1-[2-(1-phenyl-1H- pyrazol-4-yl)-1,3-thiazole-4- carbonyl]pyrrolidine-2-carboxylate |
| 134 | 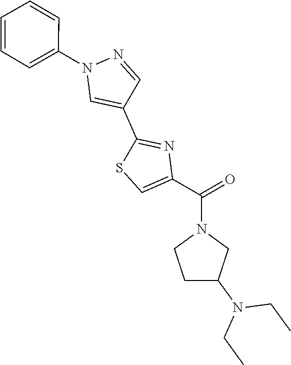 [SMILES: CCN(CC)C1CCN(C(=O)c2csc(-c3cnn(-c4ccccc4)c3)n2)C1] | N,N-diethyl-1-[2-(1-phenyl- 1H-pyrazol-4-yl)-1,3-thiazole-4- carbonyl]pyrrolidin-3-amine |
| 138 | 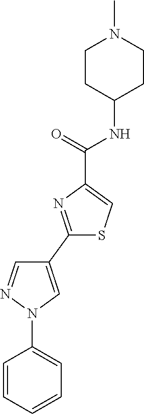 [SMILES: CN1CCC(NC(=O)c2csc(-c3cnn(-c4ccccc4)c3)n2)CC1] | N-(1-methylpiperidin-4-yl)-2-(1-phenyl- 1H-pyrazol-4-yl)-1,3-thiazole-4- carboxamide |
| 144 | 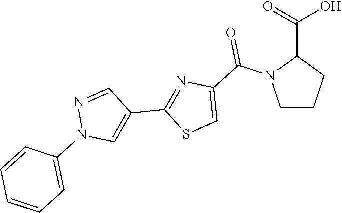 [SMILES: O=C(O)C1CCCN1C(=O)c1csc(-c2cnn(-c3ccccc3)c2)n1] | 1-[2-(1-phenyl-1H-pyrazol- 4-yl)-1,3-thiazole-4- carbonyl]pyrrolidine-2-carboxylic acid |
| 147 | 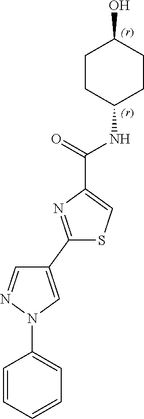 [SMILES: O=C(N[C@H]1CC[C@H](O)CC1)c1csc(-c2cnn(-c3ccccc3)c2)n1] | 2-(1-phenyl-1H-pyrazol-4-yl)-N- [(1r,4r)-4- hydroxycyclohexyl]-1,3-thiazole-4- carboxamide |
| 148 | 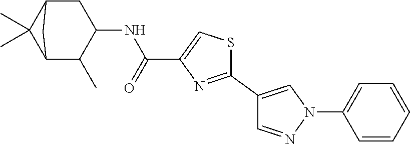 [SMILES: CC1C(NC(=O)c2csc(-c3cnn(-c4ccccc4)c3)n2)CC2CC1C2(C)C] | 2-(1-phenyl-1H-pyrazol-4-yl)-N-{2,6,6- trimethylbicyclo[3.1.1]heptan-3-yl}-1,3- thiazole-4-carboxamide |
| 151 | 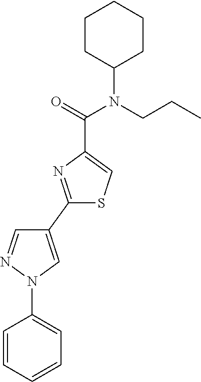 [SMILES: CCCN(C(=O)c1csc(-c2cnn(-c3ccccc3)c2)n1)C1CCCCC1] | N-cyclohexyl-2-(1-phenyl-1H-pyrazol-4- yl)-N-propyl-1,3-thiazole-4-carboxamide |
| 155 | 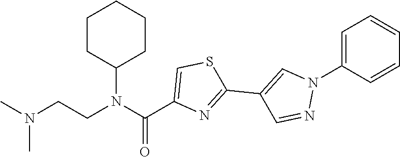 [SMILES: CN(C)CCN(C(=O)c1csc(-c2cnn(-c3ccccc3)c2)n1)C1CCCCC1] | N-cyclohexyl-N-[2- (dimethylamino)ethyl]-2-(1-phenyl-1H- pyrazol-4-yl)-1,3-thiazole-4-carboxamide |
| 157 | 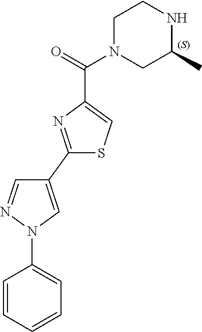 | (3S)-3-methyl-1-[2-(1- phenyl-1H-pyrazol-4-yl)-1,3-thiazole- 4-carbonyl]piperazine |
| 164 | 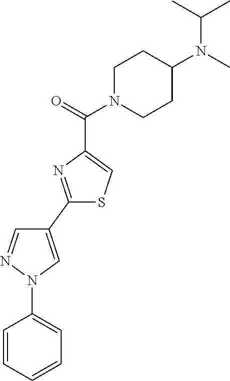 [SMILES: CC(C)N(C)C1CCN(C(=O)c2csc(-c3cnn(-c4ccccc4)c3)n2)CC1] | N-methyl-1-[2-(1-phenyl- 1H-pyrazol-4-yl)-1,3-thiazole-4- carbonyl]-N-(propan-2-yl)piperidin-4- amine |
| 166 | 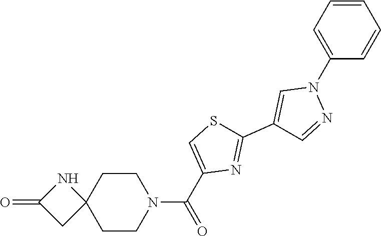 [SMILES: O=C1CC2(CCN(C(=O)c3csc(-c4cnn(-c5ccccc5)c4)n3)CC2)N1] | 7-[2-(1-phenyl-1H-pyrazol- 4-yl)-1,3-thiazole-4-carbonyl]-1,7- diazaspiro[3.5]nonan-2-one |
| 168 | 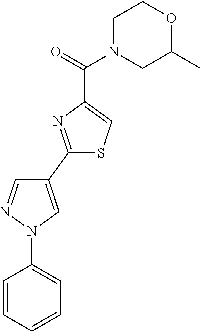 [SMILES: CC1CN(C(=O)c2csc(-c3cnn(-c4ccccc4)c3)n2)CCO1] | 2-methyl-4-[2-(1-phenyl- 1H-pyrazol-4-yl)-1,3-thiazole-4- carbonyl]morpholine |
| 169 | 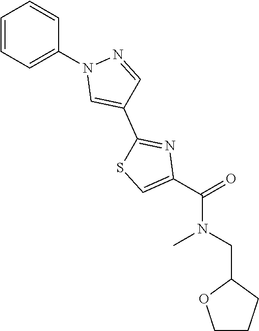 [SMILES: CN(CC1CCCO1)C(=O)c1csc(-c2cnn(-c3ccccc3)c2)n1] | N-methyl-N-[(oxolan-2- yl)methyl]-2-(1-phenyl-1H-pyrazol-4-yl)- 1,3-thiazole-4carboxamide |
| 172 | 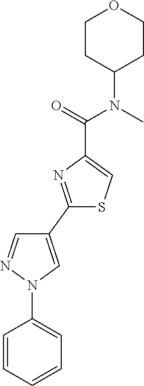 [SMILES: CN(C(=O)c1csc(-c2cnn(-c3ccccc3)c2)n1)C1CCOCC1] | N-methyl-N-(oxan-4-yl)-2-(1-phenyl-1H- pyrazol-4-yl)-1,3-thiazole-4-carboxamide |
| 173 | 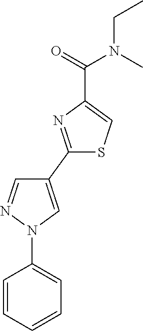 [SMILES: CCN(C)C(=O)c1csc(-c2cnn(-c3ccccc3)c2)n1] | N-ethyl-N-methyl-2-(1-phenyl-1H- pyrazol-4-yl)-1,3-thiazole-4-carboxamide |
| 174 | 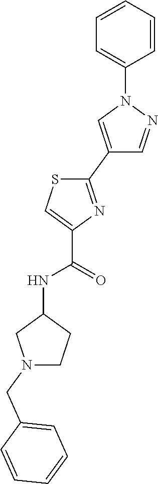 [SMILES: O=C(NC1CCN(Cc2ccccc2)C1)c1csc(-c2cnn(-c3ccccc3)c2)n1] | N-(1-benzylpyrrolidin-3-yl)-2-(1-phenyl- 1H-pyrazol-4-yl)-1,3-thiazole-4- carboxamide |
| 176 | 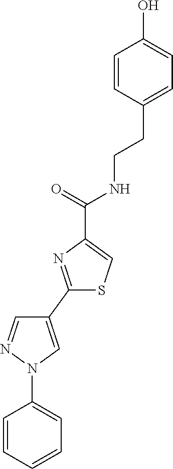 [SMILES: O=C(NCCc1ccc(O)cc1)c1csc(-c2cnn(-c3ccccc3)c2)n1] | N-[2-(4- hydroxyphenyl)ethyl]-2-(1-phenyl-1H- pyrazol-4-yl)-1,3-thiazole-4-carboxamide |
| 182 | 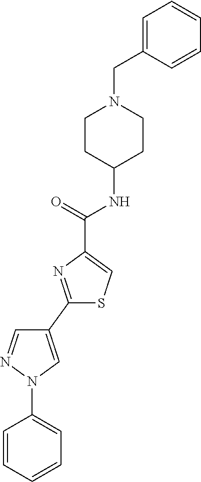 [SMILES: O=C(NC1CCN(Cc2ccccc2)CC1)c1csc(-c2cnn(-c3ccccc3)c2)n1] | N-(1-benzylpiperidin-4-yl)-2-(1-phenyl- 1H-pyrazol-4-yl)-1,3-thiazole-4- carboxamide |
| 185 | 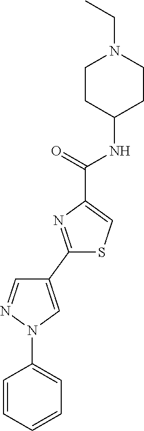 [SMILES: CCN1CCC(NC(=O)c2csc(-c3cnn(-c4ccccc4)c3)n2)CC1] | N-(1-ethylpiperidin-4-yl)-2-(1-phenyl-1H- pyrazol-4-yl)-1,3-thiazole-4-carboxamide |
| 186 | 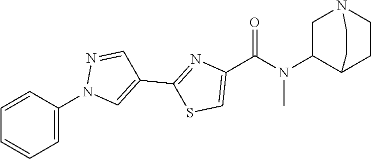 [SMILES: CN(C(=O)c1csc(-c2cnn(-c3ccccc3)c2)n1)C1CN2CCC1CC2] | N-{1- azabicyclo[2.2.2]octan-3-yl}-N-methyl- 2-(1-phenyl- 1H-pyrazol-4-yl)-1,3 thiazole-4- carboxamide |
| 187 | 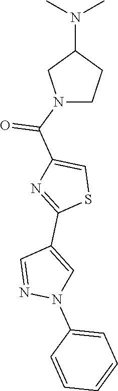 [SMILES: CN(C)C1CCN(C(=O)c2csc(-c3cnn(-c4ccccc4)c3)n2)C1] | N,N-dimethyl-1-[2-(1-phenyl-1H- pyrazol-4-yl)-1,3-thiazole-4- carbonyl]pyrrolidin-3-amine |
| 188 | 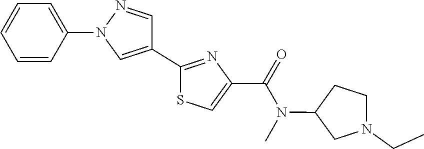 [SMILES: CCN1CCC(N(C)C(=O)c2csc(-c3cnn(-c4ccccc4)c3)n2)C1] | N-(1-ethylpyrrolidin-3-yl)-N-methyl-2- (1-phenyl-1H-pyrazol-4-yl)-1,3-thiazole- 4-carboxamide |
| 189 | 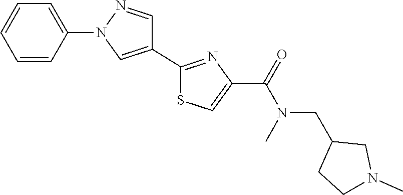 [SMILES: CN1CCC(CN(C)C(=O)c2csc(-c3cnn(-c4ccccc4)c3)n2)C1] | N-methyl-N-[(1-methylpyrrolidin-3- yl)methyl]-2-(1-phenyl-1H-pyrazol-4-yl)- 1,3-thiazole-4-carboxamide |
| 190 | 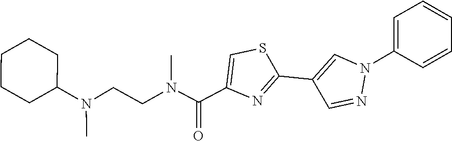 [SMILES: CN(CCN(C)C1CCCCC1)C(=O)c1csc(-c2cnn(-c3ccccc3)c2)n1] | N-{2- [cyclohexyl(methyl)amino]ethyl}-N- methyl-2-(1-phenyl-1H-pyrazol-4-yl)- 1,3-thiazole-4-carboxamide |
| 191 | 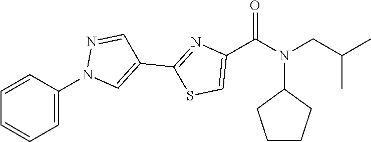 [SMILES: CC(C)CN(C(=O)c1csc(-c2cnn(-c3ccccc3)c2)n1)C1CCCC1] | N-cyclopentyl-N-(2-methylpropyl)-2-(1- phenyl-1H-pyrazol-4-yl)-1,3-thiazole-4- carboxamide |
| 192 | 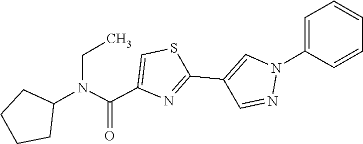 [SMILES: CCN(C(=O)c1csc(-c2cnn(-c3ccccc3)c2)n1)C1CCCC1] | N-cyclopentyl-N-ethyl-2-(1-phenyl-1H- pyrazol-4-yl)-1,3-thiazole-4-carboxamide |
| 193 | 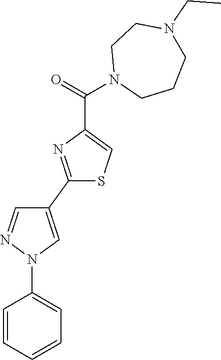 [SMILES: CCN1CCCN(C(=O)c2csc(-c3cnn(-c4ccccc4)c3)n2)CC1] | 1-ethyl-4-[2-(1-phenyl-1H-pyrazol-4-yl)- 1,3-thiazole-4 carbonyl]-1,4-diazepane |
| 194 | 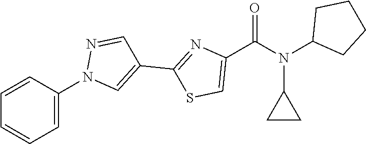 [SMILES: O=C(c1csc(-c2cnn(-c3ccccc3)c2)n1)N(C1CCCC1)C1CC1] | N-cyclopentyl-N-cyclopropyl-2-(1- phenyl-1H-pyrazol-4-yl)-1,3-thiazole- 4-carboxamide |
| 195 | 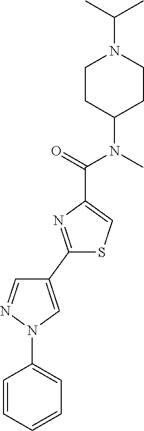 [SMILES: CC(C)N1CCC(N(C)C(=O)c2csc(-c3cnn(-c4ccccc4)c3)n2)CC1] | N-methyl-2-(1-phenyl-1H-pyrazol-4-yl)- N-[1-(propan- 2-yl)piperidin-4-yl]-1,3-thiazole-4- carboxamide |
| 196 | 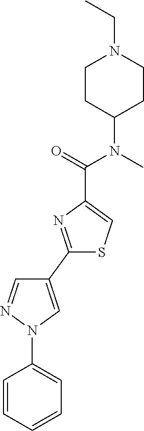 [SMILES: CCN1CCC(N(C)C(=O)c2csc(-c3cnn(-c4ccccc4)c3)n2)CC1] | N-(1-ethylpiperidin-4-yl)-N-methyl-2-(1- phenyl-1H-pyrazol-4-yl)-1,3-thiazole-4 carboxamide |
| 197 | 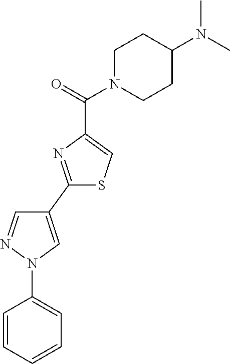 [SMILES: CN(C)C1CCN(C(=O)c2csc(-c3cnn(-c4ccccc4)c3)n2)CC1] | N,N-dimethyl-1-[2-(1-phenyl-1H- pyrazol-4-yl)-1,3-thiazole-4- carbonyl]piperidin-4-amine |
| 198 | 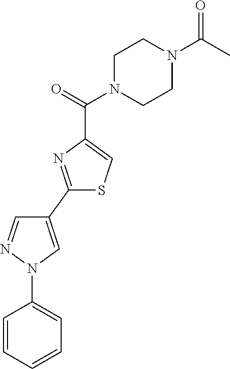 [SMILES: CC(=O)N1CCN(C(=O)c2csc(-c3cnn(-c4ccccc4)c3)n2)CC1] | 1-{4-[2-(1-phenyl-1H- pyrazol-4-yl)-1,3-thiazole-4 carbonyl]piperazin-1-yl}ethan-1-one |
| 199 | 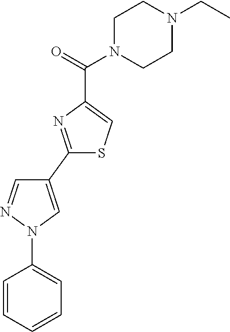 [SMILES: CCN1CCN(C(=O)c2csc(-c3cnn(-c4ccccc4)c3)n2)CC1] | 1-ethyl-4-[2-(1-phenyl-1H-pyrazol-4-yl)- 1,3-thiazole-4 carbonyl]piperazine |
| 200 | 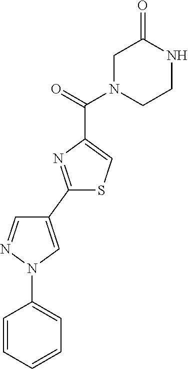 [SMILES: O=C1CN(C(=O)c2csc(-c3cnn(-c4ccccc4)c3)n2)CCN1] | 4-[2-(1-phenyl-1H-pyrazol- 4-yl)-1,3-thiazole-4- carbonyl]piperazin-2-one |
| 201 | 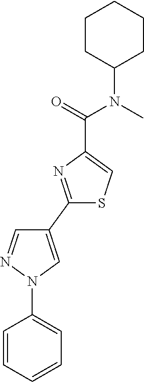 [SMILES: CN(C(=O)c1csc(-c2cnn(-c3ccccc3)c2)n1)C1CCCCC1] | N-cyclohexyl-N-methyl-2-(1-phenyl-1H- pyrazol-4-yl)-1,3-thiazole-4-carboxamide |
| 202 | 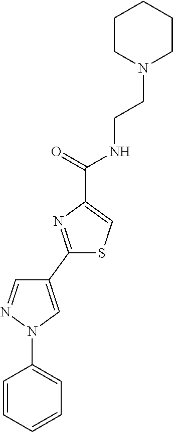 [SMILES: O=C(NCCN1CCCCC1)c1csc(-c2cnn(-c3ccccc3)c2)n1] | 2-(1-phenyl-1H-pyrazol-4-yl)-N-[2- (piperidin-1-yl)ethyl]-1,3-thiazole-4- carboxamide |
| 203 | 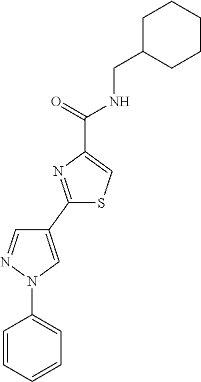 [SMILES: O=C(NCC1CCCCC1)c1csc(-c2cnn(-c3ccccc3)c2)n1] | N-(cyclohexylmethyl)-2-(1-phenyl-1H- pyrazol-4-yl)-1,3-thiazole-4-carboxamide |
| 204 | 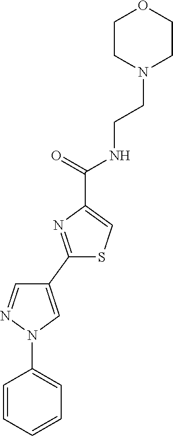 [SMILES: O=C(NCCN1CCOCC1)c1csc(-c2cnn(-c3ccccc3)c2)n1] | N-[2-(morpholin-4- yl)ethyl]-2-(1-phenyl-1H-pyrazol-4-yl)- 1,3-thiazole-4 carboxamide |
| 208 | 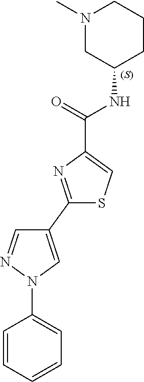 | N-[(3S)-1-methylpiperidin-3-yl]-2-(1- phenyl-1H-pyrazol-4-yl)-1,3-thiazole-4 carboxamide |
| 209 | 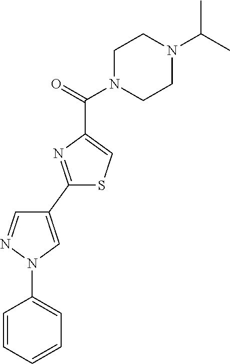 [SMILES: CC(C)N1CCN(C(=O)c2csc(-c3cnn(-c4ccccc4)c3)n2)CC1] | 1-[2-(1-phenyl-1H-pyrazol- 4-yl)-1,3-thiazole-4-carbonyl]-4- (propan-2-yl)piperazine |
| 210 | 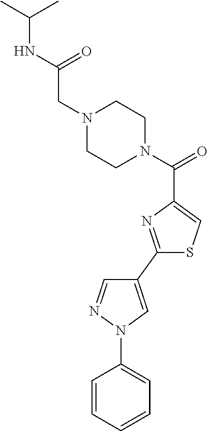 [SMILES: CC(C)NC(=O)CN1CCN(C(=O)c2csc(-c3cnn(-c4ccccc4)c3)n2)CC1] | 2-{4-[2-(1-phenyl-1H- pyrazol-4-yl)-1,3-thiazole-4 carbonyl]piperazin-1-yl}-N-(propan-2- yl)acetamide |
| 211 | 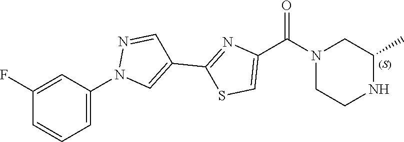 | (3S)-1-{2-[1-(3-fluorophenyl)-1H-pyrazol- 4-yl]-1,3-thiazole-4-carbonyl}-3- methylpiperazine |
| 212 | 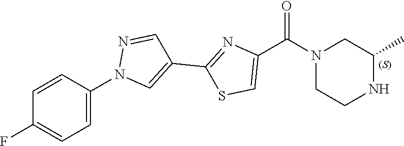 | (3S)-1-{2-[1-(4-fluorophenyl)-1H-pyrazol- 4-yl]-1,3-thiazole-4-carbonyl}-3- methylpiperazine |
| 213 | 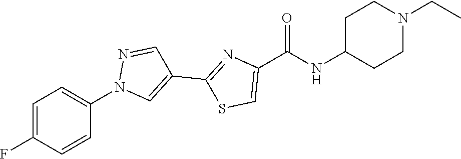 [SMILES: CCN1CCC(NC(=O)c2csc(-c3cnn(-c4ccc(F)cc4)c3)n2)CC1] | N-(1-ethylpiperidin-4-yl)-2-[1-(4- fluorophenyl)-1H-pyrazol-4-yl]-1,3- thiazole-4-carboxamide |
| 214 | 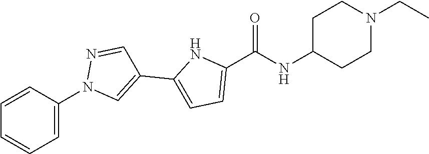 [SMILES: CCN1CCC(NC(=O)c2ccc(-c3cnn(-c4ccccc4)c3)[nH]2)CC1] | N-(1-ethylpiperidin-4-yl)-5-(1-phenyl-1H- pyrazol-4-yl)-1H-pyrrole-2-carboxamide |
| 215 | 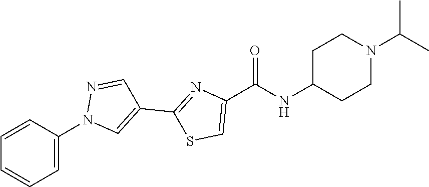 [SMILES: CC(C)N1CCC(NC(=O)c2csc(-c3cnn(-c4ccccc4)c3)n2)CC1] | 2-(1-phenyl-1H-pyrazol-4-yl)-N-[1- (propan-2-yl)piperidin-4-yl]-1,3-thiazole- 4-carboxamide |
| 216 | 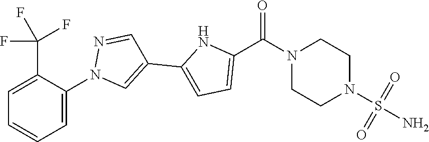 [SMILES: NS(=O)(=O)N1CCN(C(=O)c2ccc(-c3cnn(-c4ccccc4C(F)(F)F)c3)[nH]2)CC1] | 4-(5-{1-[2-(trifluoromethyl)phenyl]-1H- pyrazol-4-yl}-1H-pyrrole-2- carbonyl)piperazine-1-sulfonamide |
| 217 | 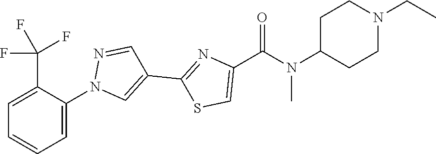 [SMILES: CCN1CCC(N(C)C(=O)c2csc(-c3cnn(-c4ccccc4C(F)(F)F)c3)n2)CC1] | N-(1-ethylpiperidin-4-yl)-N-methyl-2-{1- [2-(trifluoromethyl)phenyl]-1H-pyrazol-4- yl}-1,3-thiazole-4-carboxamide |
| 218 | 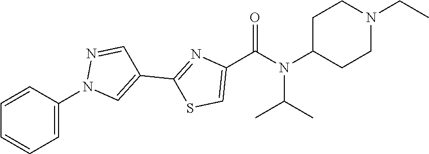 [SMILES: CCN1CCC(N(C(=O)c2csc(-c3cnn(-c4ccccc4)c3)n2)C(C)C)CC1] | N-(1-ethylpiperidin-4-yl)-2-(1-phenyl-1H- pyrazol-4-yl)-N-(propan-2-yl)-1,3- thiazole-4-carboxamide |
| 219 | 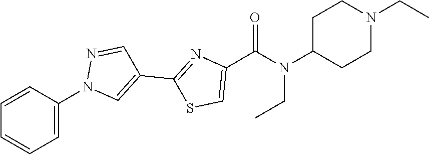 [SMILES: CCN1CCC(N(CC)C(=O)c2csc(-c3cnn(-c4ccccc4)c3)n2)CC1] | N-ethyl-N-(1-ethylpiperidin-4-yl)-2-(1- phenyl-1H-pyrazol-4-yl)-1,3-thiazole-4- carboxamide |
| 220 | 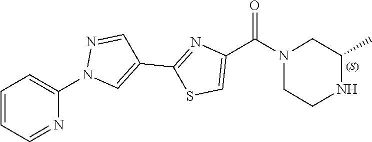 | (3S)-3-methyl-1-{2-[1-(pyridin-2-yl)-1H- pyrazol-4-yl]-1,3-thiazole-4- carbonyl}piperazine |
| 221 | 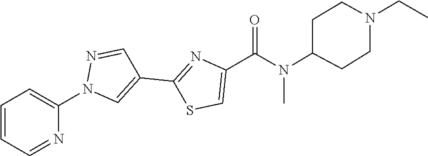 [SMILES: CCN1CCC(N(C)C(=O)c2csc(-c3cnn(-c4ccccn4)c3)n2)CC1] | N-(1-ethylpiperidin-4-yl)-N-methyl-2-[1- (pyridin-2-yl)-1H-pyrazol-4-yl]-1,3- thiazole-4-carboxamide |
| 222 | 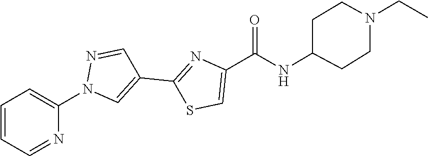 [SMILES: CCN1CCC(NC(=O)c2csc(-c3cnn(-c4ccccn4)c3)n2)CC1] | N-(1-ethylpiperidin-4-yl)-2-[1-(pyridin-2- yl)-1H-pyrazol-4-yl]-1,3-thiazole-4- carboxamide |
| 223 | 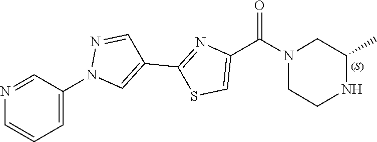 | (3S)-3-methyl-1-{2-[1-(pyridin-3-yl)-1H- pyrazol-4-yl]-1,3-thiazole-4- carbonyl}piperazine |
| 224 | 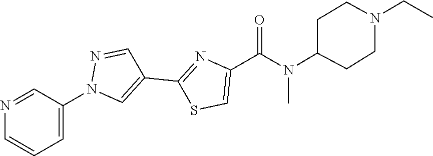 [SMILES: CCN1CCC(N(C)C(=O)c2csc(-c3cnn(-c4cccnc4)c3)n2)CC1] | N-(1-ethylpiperidin-4-yl)-N-methyl-2-[1- (pyridin-3-yl)-1H-pyrazol-4-yl]-1,3- thiazole-4-carboxamide |
| 225 | 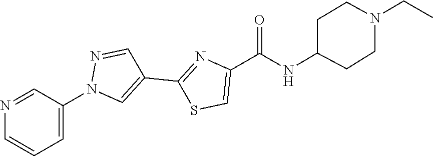 [SMILES: CCN1CCC(NC(=O)c2csc(-c3cnn(-c4cccnc4)c3)n2)CC1] | N-(1-ethylpiperidin-4-yl)-2-[1-(pyridin-3- yl)-1H-pyrazol-4-yl]-1,3-thiazole-4- carboxamide |
| 226 | 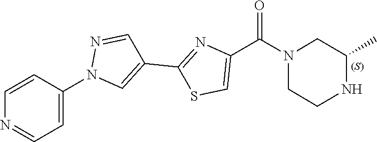 | (3S)-3-methyl-1-{2-[1-(pyridin-4-yl)-1H- pyrazol-4-yl]-1,3-thiazole-4- carbonyl}piperazine |
| 227 | 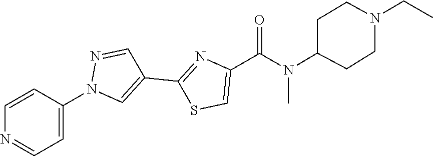 [SMILES: CCN1CCC(N(C)C(=O)c2csc(-c3cnn(-c4ccncc4)c3)n2)CC1] | N-(1-ethylpiperidin-4-yl)-N-methyl-2-[1- (pyridin-4-yl)-1H-pyrazol-4-yl]-1,3- thiazole-4-carboxamide |
| 228 | 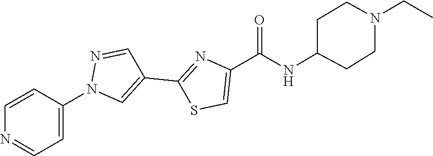 [SMILES: CCN1CCC(NC(=O)c2csc(-c3cnn(-c4ccncc4)c3)n2)CC1] | N-(1-ethylpiperidin-4-yl)-2-[1-(pyridin-4- yl)-1H-pyrazol-4-yl]-1,3-thiazole-4- carboxamide |
| 229 | 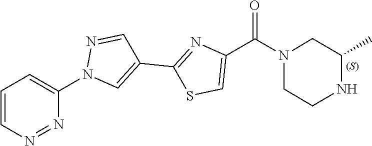 | 3-(4-{4-[(3S)-3-methylpiperazine-1- carbonyl]-1,3-thiazol-2-yl}-1H-pyrazol-1- yl)pyridazine |
| 230 | 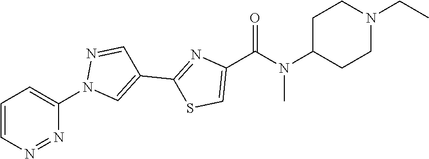 [SMILES: CCN1CCC(N(C)C(=O)c2csc(-c3cnn(-c4cccnn4)c3)n2)CC1] | N-(1-ethylpiperidin-4-yl)-N-methyl-2-[1- (pyridazin-3-yl)-1H-pyrazol-4-yl]-1,3- thiazole-4-carboxamide |
| 231 | 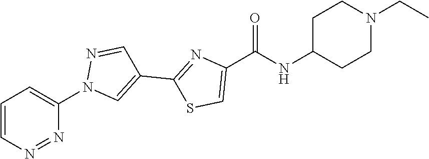 [SMILES: CCN1CCC(NC(=O)c2csc(-c3cnn(-c4cccnn4)c3)n2)CC1] | N-(1-ethylpiperidin-4-yl)-2-[1-(pyridazin- 3-yl)-1H-pyrazol-4-yl]-1,3-thiazole-4- carboxamide |
| 232 | 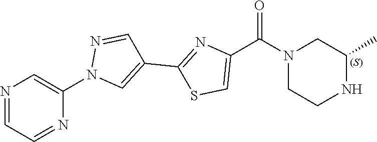 | 2-(4-{4-[(3S)-3-methylpiperazine-1- carbonyl]-1,3-thiazol-2-yl}-1H-pyrazol-1- yl)pyrazine |
| 233 | 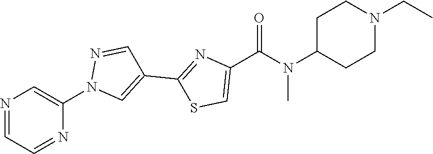 [SMILES: CCN1CCC(N(C)C(=O)c2csc(-c3cnn(-c4cnccn4)c3)n2)CC1] | N-(1-ethylpiperidin-4-yl)-N-methyl-2-[1- (pyrazin-2-yl)-1H-pyrazol-4-yl]-1,3- thiazole-4-carboxamide |
| 234 | 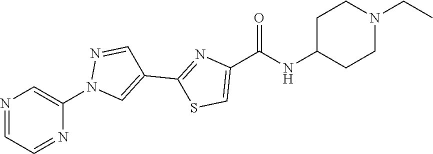 [SMILES: CCN1CCC(NC(=O)c2csc(-c3cnn(-c4cnccn4)c3)n2)CC1] | N-(1-ethylpiperidin-4-yl)-2-[1-(pyrazin-2- yl)-1H-pyrazol-4-yl]-1,3-thiazole-4- carboxamide |
| 235 | 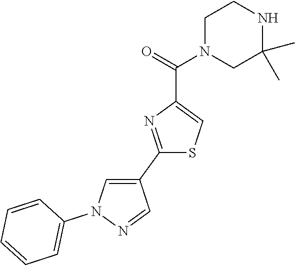 [SMILES: CC1(C)CN(C(=O)c2csc(-c3cnn(-c4ccccc4)c3)n2)CCN1] | 3,3-dimethyl-1-[2-(1-phenyl-1H-pyrazol- 4-yl)-1,3-thiazole-4-carbonyl]piperazine |
| 236 | 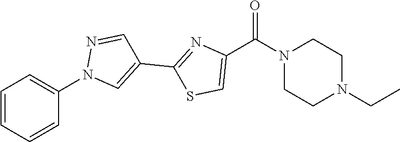 [SMILES: CCN1CCN(C(=O)c2csc(-c3cnn(-c4ccccc4)c3)n2)CC1] | 1-ethyl-4-[2-(1-phenyl-1H-pyrazol-4-yl)- 1,3-thiazole-4-carbonyl]piperazine |
| 237 | 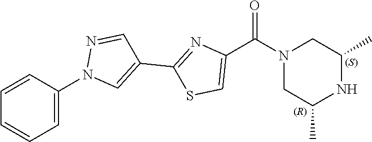 | (3R,5S)-3,5-dimethyl-1-[2-(1-phenyl-1H- pyrazol-4-yl)-1,3-thiazole-4- carbonyl]piperazine |
| 238 | 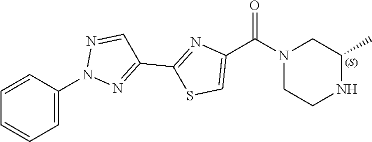 | (3S)-3-methyl-1-[2-(2-phenyl-2H-1,2,3- triazol-4-yl)-1,3-thiazole-4- carbonyl]piperazine |
| 239 | 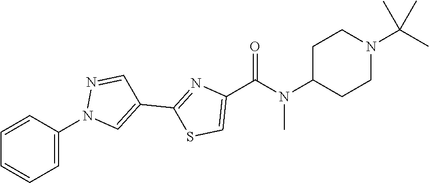 [SMILES: CN(C(=O)c1csc(-c2cnn(-c3ccccc3)c2)n1)C1CCN(C(C)(C)C)CC1] 148 | N-(1-tert-butylpiperidin-4-yl)-N-methyl-2- (1-phenyl-1H-pyrazol-4-yl)-1,3-thiazole- 4-carboxamide |
| 240 | 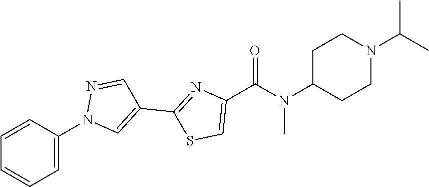 [SMILES: CC(C)N1CCC(N(C)C(=O)c2csc(-c3cnn(-c4ccccc4)c3)n2)CC1] | N-methyl-2-(1-phenyl-1H-pyrazol-4-yl)- N-[1-(propan-2-yl)piperidin-4-yl]-1,3- thiazole-4-carboxamide |
| 241 | 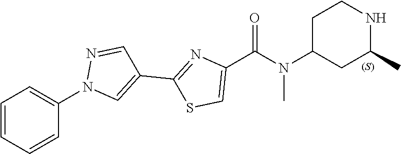 | N-methyl-N-[(2S)-2-methylpiperidin-4- yl]-2-(1-phenyl-1H-pyrazol-4-yl)-1,3- thiazole-4-carboxamide |
| 242 | 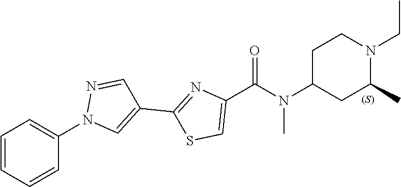 | N-[(2S)-1-ethyl-2-methylpiperidin-4-yl]- N-methyl-2-(1-phenyl-1H-pyrazol-4-yl)- 1,3-thiazole-4-carboxamide |
| 243 | 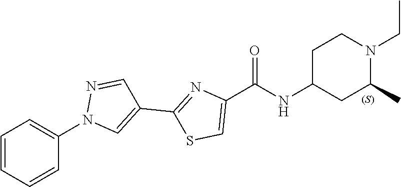 | N-[(2S)-1-ethyl-2-methylpiperidin-4-yl]-2- (1-phenyl-1H-pyrazol-4-yl)-1,3-thiazole- 4-carboxamide |
| 244 | 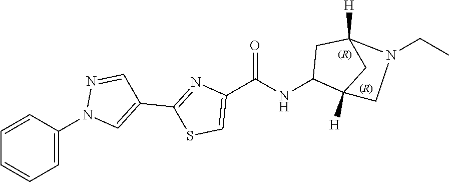 | N-[(1R,4R)-2-ethyl-2- azabicyclo[2.2.1]heptan-5-yl]-2-(1-phenyl- 1H-pyrazol-4-yl)-1,3-thiazole-4- carboxamide |
| 245 | 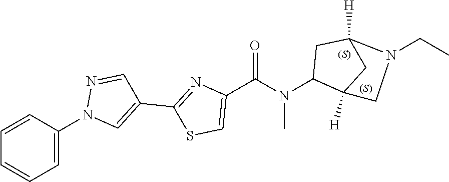 | N-[(1S,4S)-2-ethyl-2- azabicyclo[2.2.1]heptan-5-yl]-N-methyl-2- (1-phenyl-1H-pyrazol-4-yl)-1,3-thiazole- 4-carboxamide |
| 246 | 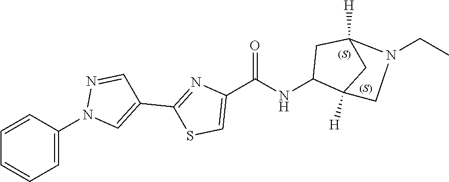 | N-[(1S,4S)-2-ethyl-2- azabicyclo[2.2.1]heptan-5-yl]-2-(1-phenyl- 1H-pyrazol-4-yl)-1,3-thiazole-4- carboxamide |
| 247 | 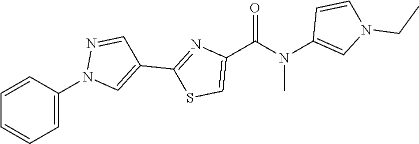 [SMILES: CCn1ccc(N(C)C(=O)c2csc(-c3cnn(-c4ccccc4)c3)n2)c1] | N-(1-ethyl-1H-pyrrol-3-yl)-N-methyl-2- (1-phenyl-1H-pyrazol-4-yl)-1,3-thiazole- 4-carboxamide |
| 248 | 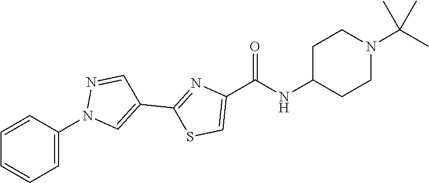 [SMILES: CC(C)(C)N1CCC(NC(=O)c2csc(-c3cnn(-c4ccccc4)c3)n2)CC1] | N-(1-tert-butylpiperidin-4-yl)-2-(1-phenyl- 1H-pyrazol-4-yl)-1,3-thiazole-4- carboxamide |
| 249 | 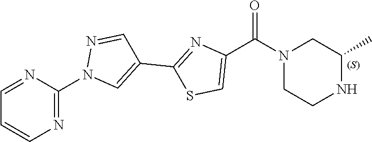 | 2-(4-{4-[(3S)-3-methylpiperazine-1- carbonyl]-1,3-thiazol-2-yl}-1H-pyrazol-1- yl)pyrimidine |
| 250 | 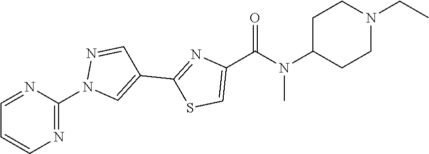 [SMILES: CCN1CCC(N(C)C(=O)c2csc(-c3cnn(-c4ncccn4)c3)n2)CC1] | N-(1-ethylpiperidin-4-yl)-N-methyl-2-[1- (pyrimidin-2-yl)-1H-pyrazol-4-yl]-1,3- thiazole-4-carboxamide |
| 251 | 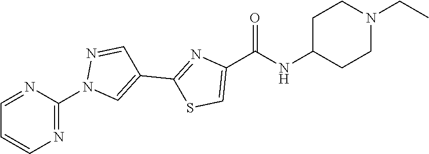 [SMILES: CCN1CCC(NC(=O)c2csc(-c3cnn(-c4ncccn4)c3)n2)CC1] | N-(1-ethylpiperidin-4-yl)-2-[1-(pyrimidin- 2-yl)-1H-pyrazol-4-yl]-1,3-thiazole-4- carboxamide |
| 252 | 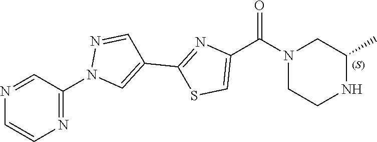 | 5-(4-{4-[(3S)-3-methylpiperazine-1- carbonyl]-1,3-thiazol-2-yl}-1H-pyrazol-1- yl)pyrimidine |
| 253 | 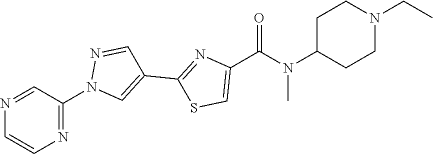 [SMILES: CCN1CCC(N(C)C(=O)c2csc(-c3cnn(-c4cnccn4)c3)n2)CC1] | N-(1-ethylpiperidin-4-yl)-N-methyl-2-[1- (pyrimidin-5-yl)-1H-pyrazol-4-yl]-1,3- thiazole-4-carboxamide |
| 254 | 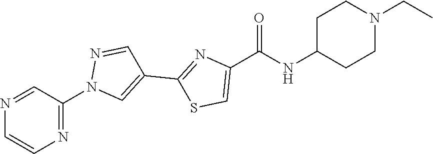 [SMILES: CCN1CCC(NC(=O)c2csc(-c3cnn(-c4cnccn4)c3)n2)CC1] | N-(1-ethylpiperidin-4-yl)-2-[1-(pyrimidin- 5-yl)-1H-pyrazol-4-yl]-1,3-thiazole-4- carboxamide |
| 255 | 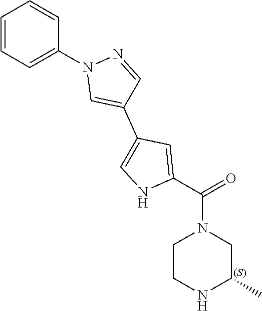 | (3S)-3-methyl-1-[4-(1-phenyl-1H-pyrazol- 4-yl)-1H-pyrrole-2-carbonyl]piperazine |
| 256 | 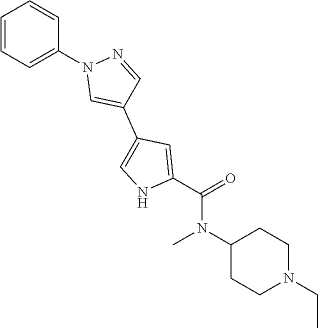 [SMILES: CCN1CCC(N(C)C(=O)c2cc(-c3cnn(-c4ccccc4)c3)c[nH]2)CC1] | N-(1-ethylpiperidin-4-yl)-N-methyl-4-(1- phenyl-1H-pyrazol-4-yl)-1H-pyrrole-2- carboxamide |
| 257 | 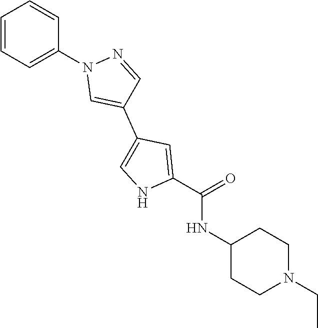 [SMILES: CCN1CCC(NC(=O)c2cc(-c3cnn(-c4ccccc4)c3)c[nH]2)CC1] | N-(1-ethylpiperidin-4-yl)-4-(1-phenyl-1H- pyrazol-4-yl)-1H-pyrrole-2-carboxamide |
| 258 | 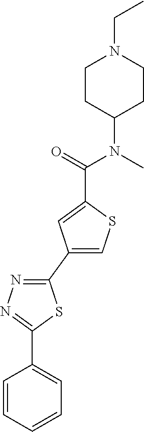 [SMILES: CCN1CCC(N(C)C(=O)c2cc(-c3nnc(-c4ccccc4)s3)cs2)CC1] | N-(1-ethylpiperidin-4-yl)-N-methyl-4-(5- phenyl-1,3,4-thiadiazol-2-yl)thiophene-2- carboxamide |
| 259 | 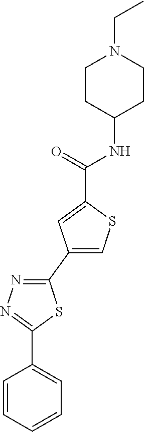 [SMILES: CCN1CCC(NC(=O)c2cc(-c3nnc(-c4ccccc4)s3)cs2)CC1] | N-(1-ethylpiperidin-4-yl)-4-(5-phenyl- 1,3,4-thiadiazol-2-yl)thiophene-2- carboxamide |
| 260 | 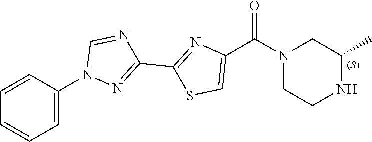 | (3S)-3-methyl-1-[2-(1-phenyl-1H-1,2,4- triazol-3-yl)-1,3-thiazole-4- carbonyl]piperazine |
| 261 | 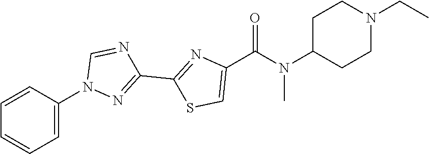 [SMILES: CCN1CCC(N(C)C(=O)c2csc(-c3ncn(-c4ccccc4)n3)n2)CC1] | N-(1-ethylpiperidin-4-yl)-N-methyl-2-(1- phenyl-1H-1,2,4-triazol-3-yl)-1,3-thiazole- 4-carboxamide |
| 262 | 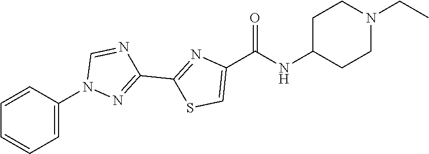 [SMILES: CCN1CCC(NC(=O)c2csc(-c3ncn(-c4ccccc4)n3)n2)CC1] | N-(1-ethylpiperidin-4-yl)-2-(1-phenyl-1H- 1,2,4-triazol-3-yl)-1,3-thiazole-4- carboxamide |
| 263 | 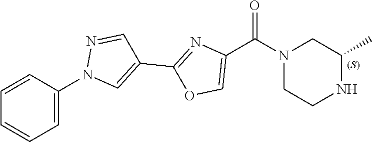 | (3S)-3-methyl-1-[2-(1-phenyl-1H-pyrazol- 4-yl)-1,3-oxazole-4-carbonyl]piperazine |
| 264 | 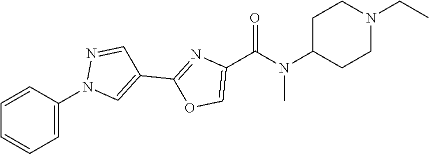 [SMILES: CCN1CCC(N(C)C(=O)c2coc(-c3cnn(-c4ccccc4)c3)n2)CC1] | N-(1-ethylpiperidin-4-yl)-N-methyl-2-(1- phenyl-1H-pyrazol-4-yl)-1,3-oxazole-4- carboxamide |
| 265 | 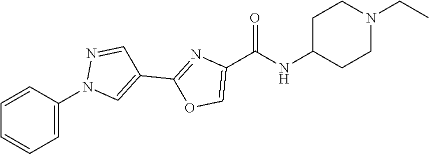 [SMILES: CCN1CCC(NC(=O)c2coc(-c3cnn(-c4ccccc4)c3)n2)CC1] | N-(1-ethylpiperidin-4-yl)-2-(1-phenyl-1H- pyrazol-4-yl)-1,3-oxazole-4-carboxamide |
| 266 | 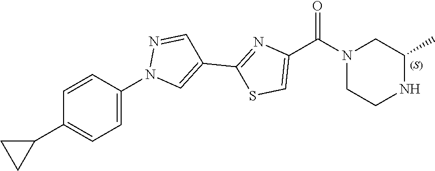 | (3S)-1-{2-[1-(4-cyclopropylphenyl)-1H- pyrazol-4-yl]-1,3-thiazole-4-carbonyl}-3- methylpiperazine |
| 267 | 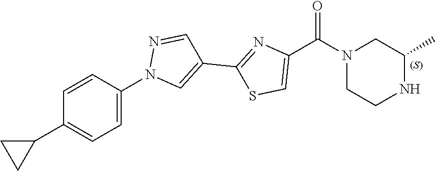 | (3S)-1-{2-[1-(4-cyclopropylphenyl)-1H- pyrazol-4-yl]-1,3-thiazole-4-carbonyl}-3- methylpiperazine |
| 268 | 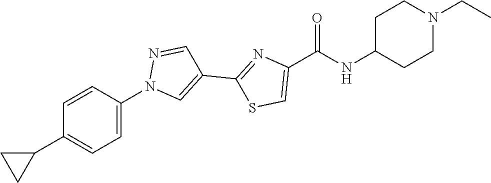 [SMILES: CCN1CCC(NC(=O)c2csc(-c3cnn(-c4ccc(C5CC5)cc4)c3)n2)CC1] | 2-[1-(4-cyclopropylphenyl)-1H-pyrazol-4- yl]-N-(1-ethylpiperidin-4-yl)-1,3-thiazole- 4-carboxamide |
| 269 | 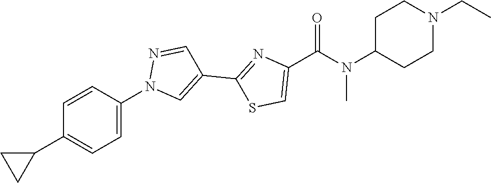 [SMILES: CCN1CCC(N(C)C(=O)c2csc(-c3cnn(-c4ccc(C5CC5)cc4)c3)n2)CC1] | 2-[1-(4-cyclopropylphenyl)-1H-pyrazol-4- yl]-N-(1-ethylpiperidin-4-yl)-N-methyl- 1,3-thiazole-4-carboxamide |
| 270 | 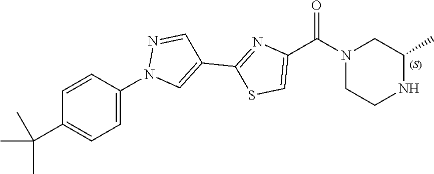 | (3S)-1-{2-[1-(4-tert-butylphenyl)-1H- pyrazol-4-yl]-1,3-thiazole-4-carbonyl}-3- methylpiperazine |
| 271 | 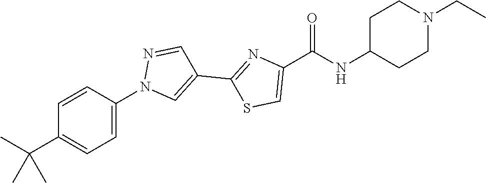 [SMILES: CCN1CCC(NC(=O)c2csc(-c3cnn(-c4ccc(C(C)(C)C)cc4)c3)n2)CC1] | 2-[1-(4-tert-butylphenyl)-1H-pyrazol-4- yl]-N-(1-ethylpiperidin-4-yl)-1,3-thiazole- 4-carboxamide |
| 272 | 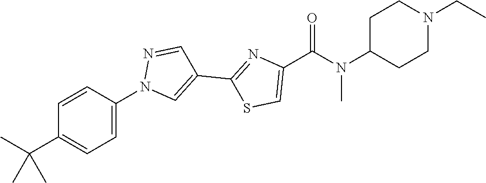 [SMILES: CCN1CCC(N(C)C(=O)c2csc(-c3cnn(-c4ccc(C(C)(C)C)cc4)c3)n2)CC1] | 2-[1-(4-tert-butylphenyl)-1H-pyrazol-4- yl]-N-(1-ethylpiperidin-4-yl)-N-methyl- 1,3-thiazole-4-carboxamide |
| 273 | 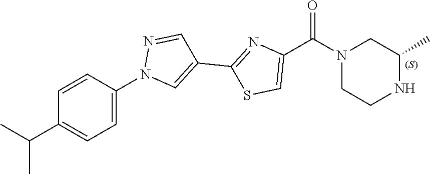 | (3S)-3-methyl-1-(2-{1-[4-(propan-2- yl)phenyl]-1H-pyrazol-4-yl}-1,3-thiazole- 4-carbonyl)piperazine |
| 274 | 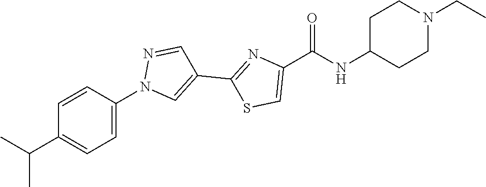 [SMILES: CCN1CCC(NC(=O)c2csc(-c3cnn(-c4ccc(C(C)C)cc4)c3)n2)CC1] | N-(1-ethylpiperidin-4-yl)-2-{1-[4-(propan- 2-yl)phenyl]-1H-pyrazol-4-yl}-1,3- thiazole-4-carboxamide |
| 275 | 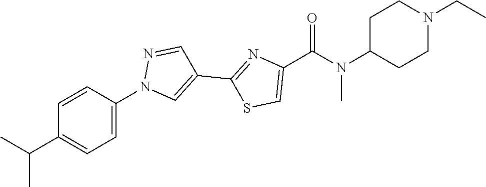 [SMILES: CCN1CCC(N(C)C(=O)c2csc(-c3cnn(-c4ccc(C(C)C)cc4)c3)n2)CC1] | N-(1-ethylpiperidin-4-yl)-N-methyl-2-{1- [4-(propan-2-yl)phenyl]-1H-pyrazol-4-yl}- 1,3-thiazole-4-carboxamide |
| 276 | 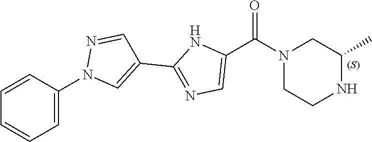 | (3S)-3-methyl-1-[2-(1-phenyl-1H-pyrazol- 4-yl)-1H-imidazole-4-carbonyl]piperazine |
| 277 | 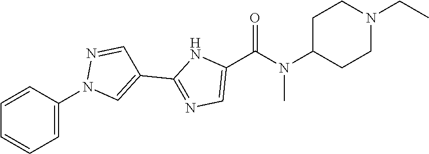 [SMILES: CCN1CCC(N(C)C(=O)c2cnc(-c3cnn(-c4ccccc4)c3)[nH]2)CC1] | N-(1-ethylpiperidin-4-yl)-N-methyl-2-(1- phenyl-1H-pyrazol-4-yl)-1H-imidazole-4- carboxamide |
| 278 | 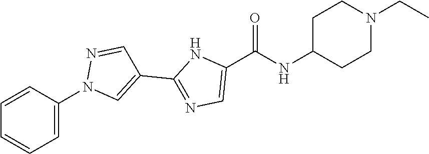 [SMILES: CCN1CCC(NC(=O)c2cnc(-c3cnn(-c4ccccc4)c3)[nH]2)CC1] | N-(1-ethylpiperidin-4-yl)-2-(1-phenyl-1H- pyrazol-4-yl)-1H-imidazole-4- carboxamide |
| 279 | 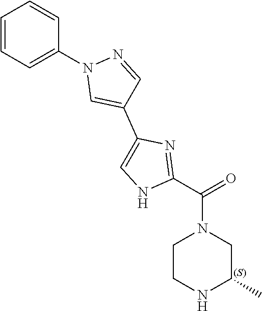 | (3S)-3-methyl-1-[5-(1-phenyl-1H-pyrazol- 4-yl)-1H-imidazole-2-carbonyl]piperazine |
| 280 | 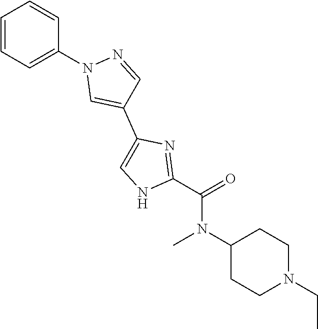 [SMILES: CCN1CCC(N(C)C(=O)c2nc(-c3cnn(-c4ccccc4)c3)c[nH]2)CC1] | N-(1-ethylpiperidin-4-yl)-N-methyl-5-(1- phenyl-1H-pyrazol-4-yl)-1H-imidazole-2- carboxamide |
| 281 | 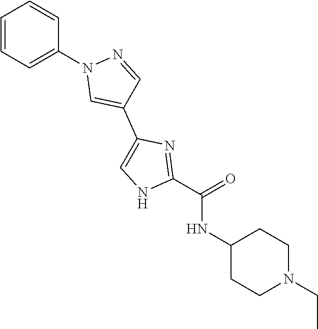 [SMILES: CCN1CCC(NC(=O)c2nc(-c3cnn(-c4ccccc4)c3)c[nH]2)CC1] | N-(1-ethylpiperidin-4-yl)-5-(1-phenyl-1H- pyrazol-4-yl)-1H-imidazole-2- carboxamide |
| 282 | 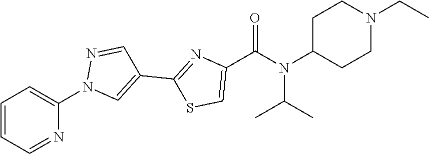 [SMILES: CCN1CCC(N(C(=O)c2csc(-c3cnn(-c4ccccn4)c3)n2)C(C)C)CC1] | N-(1-ethylpiperidin-4-yl)-N-(propan-2-yl)- 2-[1-(pyridin-2-yl)-1H-pyrazol-4-yl]-1,3- thiazole-4-carboxamide |
| 283 | 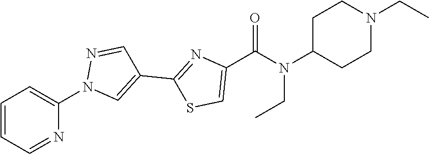 [SMILES: CCN1CCC(N(CC)C(=O)c2csc(-c3cnn(-c4ccccn4)c3)n2)CC1] | N-ethyl-N-(1-ethylpiperidin-4-yl)-2-[1- (pyridin-2-yl)-1H-pyrazol-4-yl]-1,3- thiazole-4-carboxamide |
| 284 | 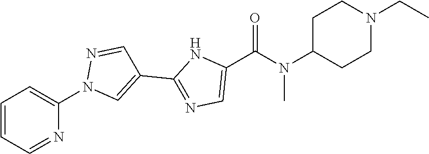 [SMILES: CCN1CCC(N(C)C(=O)c2cnc(-c3cnn(-c4ccccn4)c3)[nH]2)CC1] | N-(1-ethylpiperidin-4-yl)-N-methyl-2-[1- (pyridin-2-yl)-1H-pyrazol-4-yl]-1H- imidazole-4-carboxamide |
| 285 | 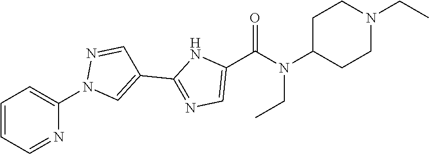 [SMILES: CCN1CCC(N(CC)C(=O)c2cnc(-c3cnn(-c4ccccn4)c3)[nH]2)CC1] | N-ethyl-N-(1-ethylpiperidin-4-yl)-2-[1- (pyridin-2-yl)-1H-pyrazol-4-yl]-1H- imidazole-4-carboxamide |
| 286 | 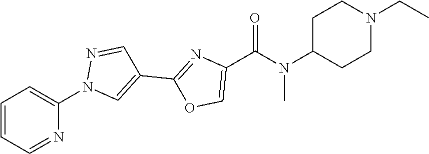 [SMILES: CCN1CCC(N(C)C(=O)c2coc(-c3cnn(-c4ccccn4)c3)n2)CC1] | N-(1-ethylpiperidin-4-yl)-N-methyl-2-[1- (pyridin-2-yl)-1H-pyrazol-4-yl]-1,3- oxazole-4-carboxamide |
| 287 | 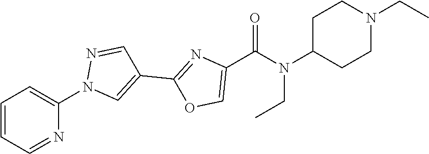 [SMILES: CCN1CCC(N(CC)C(=O)c2coc(-c3cnn(-c4ccccn4)c3)n2)CC1] | N-ethyl-N-(1-ethylpiperidin-4-yl)-2-[1- (pyridin-2-yl)-1H-pyrazol-4-yl]-1,3- oxazole-4-carboxamide |
| 288 | 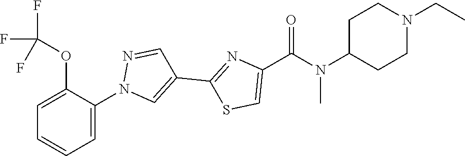 [SMILES: CCN1CCC(N(C)C(=O)c2csc(-c3cnn(-c4ccccc4OC(F)(F)F)c3)n2)CC1] | N-(1-ethylpiperidin-4-yl)-N-methyl-2-{1- [2-(trifluoromethoxy)phenyl]-1H-pyrazol- 4-yl}-1,3-thiazole-4-carboxamide |
| 289 | 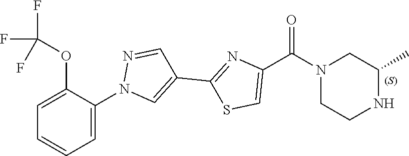 | (3S)-3-methyl-1-(2-{1-[2- (trifluoromethoxy)phenyl]-1H-pyrazol-4- yl}-1,3-thiazole-4-carbonyl)piperazine |
| 290 | 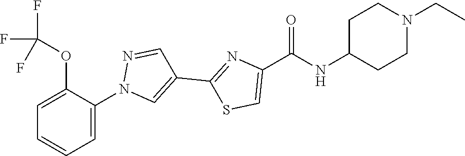 [SMILES: CCN1CCC(NC(=O)c2csc(-c3cnn(-c4ccccc4OC(F)(F)F)c3)n2)CC1] | N-(1-ethylpiperidin-4-yl)-2-{1-[2- (trifluoromethoxy)phenyl]-1H-pyrazol-4- yl}-1,3-thiazole-4-carboxamide |
| 291 | 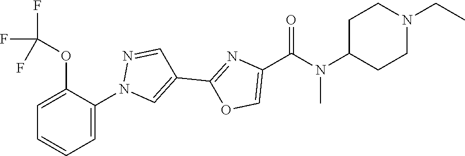 [SMILES: CCN1CCC(N(C)C(=O)c2coc(-c3cnn(-c4ccccc4OC(F)(F)F)c3)n2)CC1] | N-(1-ethylpiperidin-4-yl)-N-methyl-2-{1- [2-(trifluoromethoxy)phenyl]-1H-pyrazol- 4-yl}-1,3-oxazole-4-carboxamide |
| 292 | 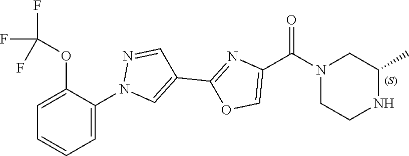 | (3S)-3-methyl-1-(2-{1-[2- (trifluoromethoxy)phenyl]-1H-pyrazol-4- yl}-1,3-oxazole-4-carbonyl)piperazine |
| 293 | 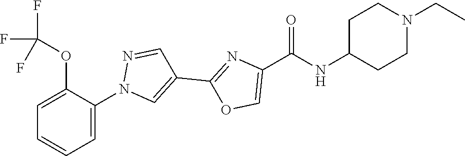 [SMILES: CCN1CCC(NC(=O)c2coc(-c3cnn(-c4ccccc4OC(F)(F)F)c3)n2)CC1] | N-(1-ethylpiperidin-4-yl)-2-{1-[2- (trifluoromethoxy)phenyl]-1H-pyrazol-4- yl}-1,3-oxazole-4-carboxamide |
| 294 | 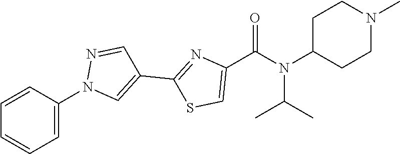 [SMILES: CC(C)N(C(=O)c1csc(-c2cnn(-c3ccccc3)c2)n1)C1CCN(C)CC1] | N-(1-methylpiperidin-4-yl)-2-(1-phenyl- 1H-pyrazol-4-yl)-N-(propan-2-yl)-1,3- thiazole-4-carboxamide |
| 295 | 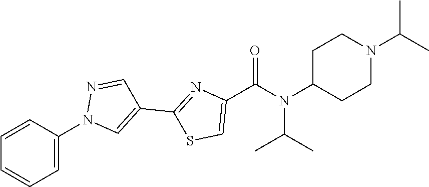 [SMILES: CC(C)N1CCC(N(C(=O)c2csc(-c3cnn(-c4ccccc4)c3)n2)C(C)C)CC1] | 2-(1-phenyl-1H-pyrazol-4-yl)-N-(propan- 2-yl)-N-[1-(propan-2-yl)piperidin-4-yl]- 1,3-thiazole-4-carboxamide |
| 296 | 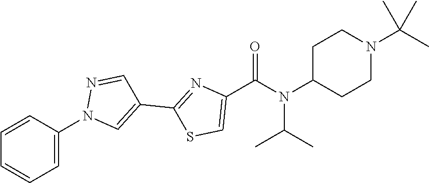 [SMILES: CC(C)N(C(=O)c1csc(-c2cnn(-c3ccccc3)c2)n1)C1CCN(C(C)(C)C)CC1] | N-(1-tert-butylpiperidin-4-yl)-2-(1-phenyl- 1H-pyrazol-4-yl)-N-(propan-2-yl)-1,3- thiazole-4-carboxamide |
| 297 | 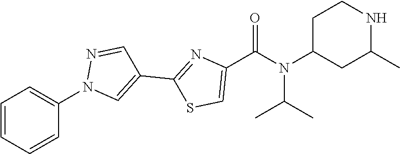 [SMILES: CC1CC(N(C(=O)c2csc(-c3cnn(-c4ccccc4)c3)n2)C(C)C)CCN1] | N-(2-methylpiperidin-4-yl)-2-(1-phenyl- 1H-pyrazol-4-yl)-N-(propan-2-yl)-1,3- thiazole-4-carboxamide |
| 298 | 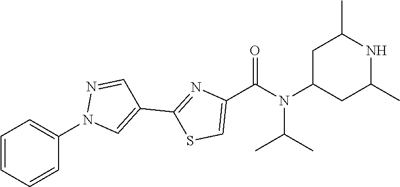 [SMILES: CC1CC(N(C(=O)c2csc(-c3cnn(-c4ccccc4)c3)n2)C(C)C)CC(C)N1] | N-(2,6-dimethylpiperidin-4-yl)-2-(1- phenyl-1H-pyrazol-4-yl)-N-(propan-2-yl)- 1,3-thiazole-4-carboxamide |
| 299 | 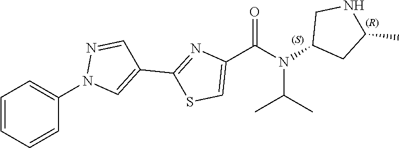 | N-[(3S,5R)-5-methylpyrrolidin-3-yl]-2-(1- phenyl-1H-pyrazol-4-yl)-N-(propan-2-yl)- 1,3-thiazole-4-carboxamide |
| 300 | 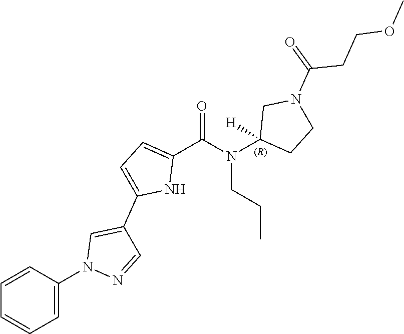 | N-[(3R)-1-(3- methoxypropanoyl)pyrrolidin-3-yl]-5-(1- phenyl-1H-pyrazol-4-yl)-N-propyl-1H- pyrrole-2-carboxamide |
| 301 | 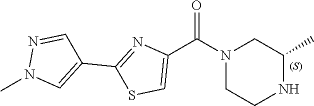 | (3S)-3-methyl-1-[2-(1-methyl-1H-pyrazol- 4-yl)-1,3-thiazole-4-carbonyl]piperazine |
| 302 | 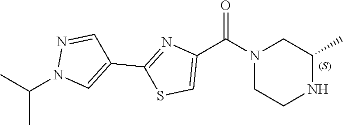 | (3S)-3-methyl-1-{2-[1-(propan-2-yl)-1H- pyrazol-4-yl]-13-thiazole-4- carbonyl}piperazine |
| 303 | 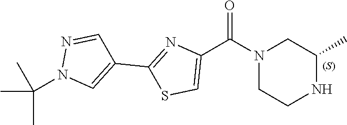 | (3S)-1-[2-(1-tert-butyl-1H-pyrazol-4-yl)- 1,3-thiazole-4-carbonyl]-3- methylpiperazine |
| 304 | 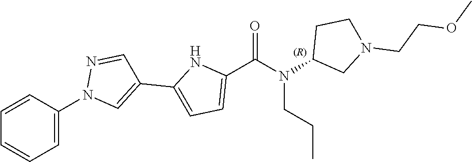 | N-[(3R)-1-(2-methoxyethyl)pyrrolidin-3- yl]-5-(1-phenyl-1H-pyrazol-4-yl)-N- propyl-1H-pyrrole-2-carboxamide |

### Biological Assays and Data

As stated above, the compounds of the present invention induce and/or stimulate autophagy and are useful in treating autophagy-related diseases. The biological activity of the compounds of the present invention can be determined by any appropriate test to determine the ability to induce and/or stimulate autophagy.

### Assessment of Stimulation of Autophagy

Compounds of this invention were assessed for their ability to stimulate autophagy using one, or more, of the assays described below

### Assay to Measure Lysosome and Autolysosome Formation

Lysosomes play a fundamental role in the autophagic pathway by fusing with autophagosomes and creating ‘autolysosomes’ in order to digest their contents. Stimulation of lysosome and autolysosome formation by a compound is indicative of a stimulation of autophagy. The ability of compounds to stimulate lysosome and autolysosome formation, and thus autophagy, in live cells was assessed via fluorescent microscopy using various fluorescent stains for labelling and tracking acidic organelles (including lysosomes and autolysosomes) such as: LysoView™ 650 (70059 and 70059-T, Biotium), Lyso View™633 (70058 and 70058-T, Biotium) and LysoTracker™ Deep Red (L12492, ThermoFisher Scientific). The cellular phenotype was quantitatively assessed for the induction of acidic vesicle formation and compared to a non-treated control, thus providing a measure of the ability of the compound under investigation to stimulate autophagy.

Representative procedure using LysoView™ 633 dye or LysoTracker™ Deep Red dye: Human osteosarcoma U2OS cells (40,000 cells/well) were seeded in a 24 well glass bottom plate (Sensoplate, Greiner Bio-One) and were incubated overnight in a humidified atmosphere at 37° C. and 5% CO2. Cells were grown in DMEM (Gibco) supplemented with 10% Fetal Bovine Serum (FBS) and 100 units/ml penicillin and 100 μg/ml streptomycin (Invitrogen). After the attachment period, cells were treated with different compounds of interest (at various concentrations in DMSO) or DMSO (non-treated control) in cell culture medium and were incubated for 24 hours. Compound-containing medium was removed and cells were incubated with pre-warmed cell culture medium containing 1× Lyso View™ 633 (70058 and 70058-T, Biotium) or 50 nM LysoTracker™ Deep Red (L12492, ThermoFisher Scientific) for 45 minutes at 37° C. Finally, cell nuclei were stained for 10 minutes using Hoechst 33342 (1 μg/mL) and then the medium was replaced with fresh medium. The 24 well plate was then fitted into a heated stage on the microscope and cells maintained at 37° C. Images were captured using appropriate filter set for Cy5 and DAPI detection with the EVOS M7000 Microscope (ThermoFisher Scientific). The cellular phenotype was visually assessed for the induction of acidic vesicle formation relative to the non-treated control; multiple images were acquired and analysed using ImageJ or Cell Profiler.

All of compounds 1-304 according to the invention showed clearly increased acidic vesicle formation in human osteosarcoma U2OS cells relative to a DMSO-treated control (non-treated control) at a concentration of 10 μM, except compounds 223, 226, 227, 232, 241, 258 and 259, which showed only a slight increase. In this assay, acidic vesicle formation was increased by at least 25% for compounds 1-304, except compounds 223, 226, 227, 232, 241, 258 and 259, which showed only a slight increase. This is evidence for a significant stimulation of autophagy by these compounds under these conditions.

Selected compounds of this invention were also tested and showed increased acidic vesicle formation in human osteosarcoma U2OS cells relative to a DMSO-treated control (non-treated control) at a concentration of 0.8 μM. This is evidence for significant stimulation of autophagy by the compounds of this invention, and particularly by these compounds under the conditions as tested. Table 2 shows this data for the selected compounds. Compounds having an activity designated as “+” provided a percentage increase in acidic vesicle formation of between 10% and 30% over the DMSO-treated control. Compounds having an activity designated as “++” provided a percentage increase in acidic vesicle formation of between 30% and 50% over the DMSO-treated control. Compounds having an activity designated as “+++” provided a percentage increase in acidic vesicle formation of between 50% and 100% over the DMSO-treated control. Compounds having an activity designated as “++++” provided a percentage increase in acidic vesicle of greater than 100% over the DMSO-treated control. Preferred compounds are shown in Table 3.

TABLE 3

| Compound | | Compound | | Compound | |
|---|---|---|---|---|---|
| No. | Activity | No. | Activity | No. | Activity |
| 190 | + | 216 | + | 239 | + |
| 196 | ++ | 217 | + | 244 | + |
| 198 | + | 221 | + | 245 | + |
| 202 | ++ | 223 | + | 246 | + |
| 204 | + | 225 | + | 249 | + |
| 208 | + | 228 | + | 250 | + |
| 213 | + | 231 | + | 258 | + |
| 214 | + | 236 | + | 259 | + |

| Compound | | Compound | |
|---|---|---|---|
| No. | Activity | No. | Activity |
| 218 | ++ | 255 | +++ |
| 229 | ++ | 257 | +++ |
| 234 | ++ | 219 | ++++ |
| 235 | ++ | | |
| 256 | ++ | | |
| 224 | +++ | | |
| 232 | +++ | | |
| 233 | +++ | | |

Certain compounds of this invention also showed increased acidic vesicle formation other cell types, relative to a DMSO-treated control (non-treated control). For example, certain compounds of this invention were found to show increased acidic vesicle formation relative to a DMSO-treated control (non-treated control) in SH-SY5Y cells (Table 4). This is evidence for significant stimulation of autophagy by this compound under these conditions. SH-SY5Y cells are a human-derived neuroblastoma cell line widely used in in vitro models of neurological disorders (Ross R A, Spengler B A, Biedler J L. Coordinate morphological and biochemical interconversion of human neuroblastoma cells. J Natl Cancer Inst. 1983 October; 71 (4): 741-7. PMID: 6137586).

A brief description of the procedure employed is outlined here:
Day 1: Cells were seeded at 15K cells/well in the middle 60 wells of 6×96 well Cellvis imaging plates
Day 2: Media was replaced with 90 μl of DMEM/F12 (+10% FBS/1% P/S) containing NucBlue Live and the cells were treated with 10 μl of compound (10× concentrated) in triplicate for 24 hours.
Day3: Treatment was removed and cells were incubated for 30 minutes with LysoTracker™ Deep Red dye. Cells were washed twice with media and immediately imaged on EVOS M7000 at ×20 (6 images per well, 30 minutes imaging time/plate). Images were analysed using Celleste Image analysis software.

TABLE 4
Fold-change in acidic vesicle formation relative to DMSO-treated
control (non-treated control) in SH-SY5Y cells

| | Fold change at compound | Fold change at compound |
|---|---|---|
| Compound | concentration of 0.8 μM | concentration of 10 μM |
| 3 | 1.6 | 3.5 |
| 218 | 1.6 | 4.8 |
| 238 | 2.1 | 2.9 |
| 248 | 2.2 | 1.9 |
| 256 | 1.8 | 2.7 |
| 257 | 1.9 | 2.6 |
| 259 | 2.3 | 3.6 |

Based on these results this is evidence that these compounds induce autophagy. Additional evidence that this activity will be therapeutic in various autophagy-related diseases are disclosed in the following examples.

### Tandem Reporter Assay (Assay to Assess Autophagic Flux)

Selected compounds of this invention were also assessed for their ability to stimulate autophagy using a U2OS cell line stabling expressing RFP-cGFP-hLC3b (a tandem reporter cell line).

During the process of autophagy, the cargo to be degraded is first enveloped by organelles called autophagosomes. These autophagosomes then fuse with lysosomes, causing them to become acidified and their digestive enzymes to become activated. The term “autophagic flux” is used to represent the dynamic process of autophagy: autophagic flux refers to the whole process of autophagy, including autophagosome formation, maturation, fusion with lysosomes, subsequent breakdown and the release of macromolecules back into the cytosol (Zhang X J, Chen S, Huang K X, Le W D. Why should autophagic flux be assessed? Acta Pharmacol Sin. 2013 May; 34 (5): 595-9. doi: 10.1038/aps.2012.184. Epub 2013 Mar. 11). Compounds that stimulate autophagic flux stimulate the dynamic process of autophagy (the whole process of autophagy).

LC3b is a protein found in the membrane of autophagosomes which has been used to generate genetic reporters of autophagy in cells. In these systems a tandem fusion of LC3b is engineered with two fluorescent proteins of different wavelengths: one which is acid sensitive (for example eGFP, which fluoresces green) and the other which is acid insensitive (for example RFP, which fluoresces red). Cells expressing RFP-cGFP-hLC3b can be examined using fluorescent microscopy: the autophagosomes present will fluoresce in both channels (either yellow in an overlay of both channels or puncta that are present in both the individual red and green channels) but the autolysosomes present will fluoresce in the RFP (red) channel only. Using a fluorescent microscope, the number of autophagosomes and autolysosomes can be counted, allowing for the monitoring of both the induction of autophagy (total puncta count) and the rate of autophagic flux (ratio of red-only to red-and-green puncta). The influence of small molecules on autophagy induction and flux can be assessed using this system by comparison to a non-treated control.

Representative Procedure: Human osteosarcoma U2OS cells stably expressing RFP-eGFP-hLC3b were seeded (10,000 cells/well) into a 96-well glass-bottom plate (Cell Carrier Ultra, Perkin Elmer) in DMEM Glutamax media (Gibco) supplemented with 10% Fetal Bovine Serum (FBS) and antibiotics (100 u/ml penicillin, 100 μg/ml streptomycin, Invitrogen), then incubated overnight in a humified atmosphere at 37° C. and 5% CO2. The cells were then treated with the compounds of interest, dissolved in DMSO, in a duplicated 5- or 10-point dose-response. 8 wells were treated with an equivalent volume of DMSO, 4 with 0.3 M torin-1 as a positive control (MCE) and 4 wells with 0.4 μM torin-1 plus 0.2 μM bafilomycin (MCE) as a control for flux inhibition. The cells were returned to the incubator and the treatment continued for 24 hours. After treatment, the media was aspirated off and the cells fixed with a solution of 4% formaldehyde+1% glutaraldehyde (Sigma) in dPBS (plus magnesium and calcium, Gibco). Fixation was allowed to proceed for 15 minutes at room temperature then the fixative solution discarded and replaced with PBS (Gibco) plus Hoescht 33342 (1 μg/ml, Sigma). After 30 minutes the plates were imaged on an Opera Phenix confocal microscope (Perkin-Elmer) using a 40× water objective, collecting using DAPI, mCherry and GFP channels. Cells were detected based upon the staining of their nuclei with DAPI using the Harmony image analysis software (Perkin-Elmer) and the number of autophagosomes (GFP and RFP puncta) and autolysosomes (RFP only puncta) per cell were counted using the spot picking function within the software.

Selected compounds of this invention showed an increase in the number of RFP-only puncta (autolysosomes) relative to a DMSO-treated control (non-treated control) at different concentrations (Table 5). This is evidence for significant stimulation of autophagic flux (and thus the whole process of autophagy) by the compounds of this invention, and particularly by these compounds under the conditions as tested.

TABLE 5
Change in the number of RFP-only puncta (autolysosomes) relative to
a DMSO-treated control (non-treated control) in a U2OS cell line
stabling expressing RFP-eGFP-hLC3b (a tandem reporter cell line)
“Autolysosome % DMSO activity” is the activity of the compound
(based on the number of RFP-only puncta ((autolysosomes)) as a
percentage of the activity of the DMSO-treated control (non-treated
control).

| | Autolysosome % DMSO | Autolysosome % DMSO |
|---|---|---|
| | activity (compound | activity (compound |
| Compound | concentration of 10 μM) | concentration of 1 μM)) |
| 299 | 152 | 162 |
| 298 | 151 | 187 |
| 297 | 119 | 109 |
| 273 | — | 224 |
| 270 | — | 251 |
| 279 | 158 | 123 |
| 266 | 273 | 176 |

### Assessment of Activity in Models of Various Diseases and Conditions

### Example 1

### Activity in a Cellular Model of Parkinson's Disease

Compounds 144, 157 (particularly preferred), 185 and 196 were selected as example compounds of this invention to be evaluated in this model.

This model (run by Neuro-Sys SAS, France) is based on a primary culture of dopaminergic Tyrosine hydroxylase (TH)-positive neurons injured with the dopaminergic neurons-specific toxin (DA-toxin) 1-methyl-4-phenyl-1,2,3,6 tetrahydropyridine (MPTP, a prodrug of MPP⁺) (Dauer and Przedborski, 2003). Any substances reducing DA-toxin neurotoxicity may be useful as a new therapeutic agent for the treatment or prevention of PD.

### Methods

Primary culture of mesencephalic neurons: Rat dopaminergic neurons were cultured as described by Visanji et al., 2008 and Callizot et al., 2019. Briefly, pregnant female rat (Wistar) of 15 days of gestation were killed using a deep anesthesia with CO2 chamber and a cervical dislocation. The midbrains obtained from 15-day-old rat embryos (Janvier, France) were dissected under a microscope. The embryonic midbrains will be removed and placed in ice-cold medium of Leibovitz (L15) containing 2% of Penicillin-Streptomycin (PS) and 1% of bovine serum albumin (BSA). The ventral portion of the mesencephalic flexure, a region of the developing brain rich in dopaminergic neurons, was used for the cell preparations.

The midbrains were dissociated by trypsinisation for 20 min at 37° C. (solution at a final concentration of 0.05% trypsin and 0.02% EDTA). The reaction was stopped by the addition of Dulbecco's modified Eagle's medium (DMEM) containing DNAase I grade II (0.5 mg/mL) and 10% of foetal calf serum (FCS). Cells were then mechanically dissociated by 3 passages through a 10 ml pipette. Cells were then centrifuged at 180×g for 10 min at +4° C. on a layer of BSA (3.5%) in L15 medium. The supernatant was discarded, and the cell pellets re-suspended in a defined culture medium consisting of Neurobasal supplemented with B27 (2%), L-glutamine (2 mM) and 2% of PS solution and 10 ng/mL of Brain-derived neurotrophic factor (BDNF) and 1 ng/ml of Glial-Derived Neurotrophic Factor (GDNF). Viable cells were counted in a Neubauer cytometer using the trypan blue exclusion test. The cells were seeded at a density of 40,000 cells/well in 96 well-plates (pre-coated with poly-L-lysine) and maintained in a humidified incubator at 37° C. in 5% CO2/95% air atmosphere. Half of the medium was changed every 2 days with fresh medium. The wells of first and last lines and columns were used (to avoid any edge effect) and were be filled with sterile water.

### Test Compounds and MPTP:

i) Pre-incubation. On day 6 of culture, the test compounds were dissolved in PBS or DMSO and incubated for 1 hour before the MPTP exposure.
ii) Injury. One hour after the application of test compound, MPTP was added to a final concentration of 4 μM, diluted in control medium still in presence of compounds/inhibitors for 48 h. Test compounds were tested on one culture in 96-well plate (n=6 culture wells per condition).

### Endpoint Evaluation:

Immunostaining: TH and α-Synuclein. 48 Hours after intoxication, the cell culture supernatant was removed, and the cells were fixed by a solution of 4% paraformaldehyde in PBS, pH=7.3 for 20 min at room temperature. The cells were washed twice in PBS, and then permeabilized. Non-specific sites were blocked with a solution of PBS containing 0.1% of saponin and 1% FCS for 15 min at room temperature. The cultures were incubated with: (a) monoclonal anti-Tyrosine Hydroxylase (TH) antibody produced in mouse at dilution of 1/10000 in PBS containing 1% FCS, 0.1% saponin, for 2 hours at room temperature, and (b) polyclonal anti-alpha synuclein (α-synuclein) antibody produced in rabbit at dilution of 1/200 in PBS containing 1% FCS, 0.1% saponin, for 2 h at room temperature. These antibodies were revealed with Alexa Fluor 488 goat anti-mouse IgG at the dilution 1/800 and with Alexa Fluor 568 goat anti-rabbit IgG at the dilution 1/400 in PBS containing 1% FCS, 0.1% saponin, for 1 h at room temperature.

Automatic computer analysis. For each condition, 20 pictures (representing the whole well area) were automatically taken using ImageXpress® (Molecular Devices) at 10× magnification (20 pictures, for TH and α-synuclein into TH neurons) using the same acquisition parameters. From images, analyses was directly and automatically performed by MetaXpress® (Molecular Devices).

The following read-outs were measured:
i) Analysis of total number of TH neurons (dopaminergic TH positive neurons)
ii) Total neurite network of dopaminergic TH positive neurons (in μm)
iii) α-Synuclein aggregation (overlapping between TH and α-syn staining)

Compounds 144, 156, 157, 171, 185 and 196 showed positive effects in this in vitro model of PD based on MPP+ injury.

Compound 144 significantly protected the dopaminergic neurons at a concentration of 100 nM (a 25% increase in the number of dopaminergic TH positive neurons compared to MPTP injury alone). This compound lowered α-Synuclein aggregation at concentrations of 1 μM, 500 nM and 100 nM (a >15% decrease in α-syn area per dopaminergic TH positive neuron compared to MPP+ injury alone).

Compound 157 showed significant positive effects at a concentration of 1 μM on all the three read-outs: (i) a 27% increase in the number of dopaminergic TH postive neurons compared to MPP+ injury alone, (ii) a 35% increase in the length of the neurite network of dopaminergic TH positive neurons compared to the MPP+ injury alone, and (iii) a 21% decrease in α-syn area per dopaminergic TH positive neuron compared to MPP+ injury alone.

Compound 185 displayed a large and significant neuroprotective effect at concentrations of 500 nM, 1 μM and 10 μM: (i) a >22% in increase in the number of dopaminergic TH positive neurons compared to MPP+ injury alone and (ii) a >27% increase in the length of the neurite network of dopaminergic TH positive neurons compared to the MPP+ injury alone. This effect was correlated with a decrease of α-Synuclein aggregation (a >14% decrease in α-syn area per dopaminergic TH positive neuron compared to MPP+ injury alone was observed).

For Compound 196, a significant neuroprotective effect was observed at a concentration of 500 nM (a 26% increase in the number of dopaminergic TH positive neurons compared to MPP+ injury alone). The compound lowered α-Synuclein aggregation at concentrations of 1 μM, 500 nM and 100 nM (decreases in α-syn area per dopaminergic TH positive neuron compared to MPP+ injury alone of 19%, 35% and 26% respectively).

This assay is an in vitro model of Parkinson's disease. Activity in this model (positive effects) strongly support that compounds of this invention are efficacious in the treatment of Parkinson's disease and other autophagy-related neurodegenerative diseases or conditions.

### REFERENCES

Callizot N, Combes M, Henriques A, Poindron P. Necrosis, apoptosis, necroptosis, three modes of action of dopaminergic neuron neurotoxins. PLOS ONE 14 (4): e0215277
Dauer W., Przedborski S. Parkinson's disease: mechanisms and models. Neuron 2003, 39 (6): 889-909
Visanji N P, Orsi A, Johnston T H, Howson P A, Dixon K, Callizot N, Brotchie J M and Rees D D. PYM50028, a novel, orally active, nonpeptide neurotrophic factor inducer, prevents and reverses neuronal damage induced by MPP+ in mesencephalic neurons and by MPTP in a mouse model of Parkinson's disease. FASEB J. 2008; 22 (7): 2488-97

### Example 2

### Demonstration of Activity in a Cellular Model of Amyotrophic Lateral Sclerosis (ALS)

Compounds 157 (particularly) preferred), 185 and 196 were selected example compounds if this invention to evaluate in this model. This model (run by Neuro-Sys SAS, France) evaluates the protective effects of test compounds on primary spinal cord lower motor neurons (MNs) from SOD1 G93A Tg rats, after a glutamate injury. The MNs are pre-treated with the test compounds for 1 hour prior to exposure to glutamate, and readouts are measured 24 hours later. This model functions as an in vitro model of Amyotrophic Lateral Sclerosis. In this model, the glutamate injury leads to: (i) a decrease in the number of MNs, (ii) a reduction in the length of the motor neuron network, and (iii) an increase in the cytoplasmic area of Transactivating response element DNA binding protein 43 kDa (TDP43)=relative to a non-injured control (TDP43 has been shown to accumulate in the cytpoplasm of motor neurons in most cases of ALS). Any substances which can attenuate one or more of these effects can be considered to have protective effects in the model and may be useful as a new therapeutic agent for the treatment or prevention of ALS.

### Methods

Genotyping of SOD1 Tg embryos: Pregnant female rat SODIG93A (Sprague Dawley, Taconic), of 14 days of gestation were obtained fromTaconic Bioscience. On the day of the dissection (from pregnant females at 14 days of gestation), a piece of each embryo brain (˜3 mm) was placed in a 2 mL tube free DNase with a new scalpel. The DNA was extracted with the SYBR Green Extract-N-Amp tissue PCR kit (Sigma Aldrich). Briefly, 120 μL of extraction solution was put on each piece of embryo heads. Then, they were incubated for 10 min at room temperature. At the end of this incubation period, the heads were incubated for 5 min at 95° C. Immediately after this last incubation, 100 μL of neutralizing solution was added; each DNA extract was diluted at 1/40 and stored at +4° C. until use. SOD1G93A gene was determined using genomic fragment with human SOD1 primers (5′-CATCAGCCCTAATCCATCTGA-3′ (SEQ ID NO: 1); 5′-CGCGACTAACAATCAAAGTGA-3′ (SEQ ID NO: 2)). The SOD1 primers were diluted at 3 μM in sterile ultrapure water. Briefly, a mix for PCR was prepared with ultrapure water (4 μL per sample), primer at 3 μM (2 μL per sample) and Master Mix (10 μL per sample). In a PCR 96 wells plate, 16 μL of PCR mix was added in each well. 4 μL of each diluted DNA was added according to a plan deposit. The RT-PCR was ran using the CFX96 Biorad RT-PCR system, using the following program: Initial denaturation (95° C., 20 sec)/45 cycles (95° C., 10 sec; 65° C., 10 sec; 72° C., 30 sec)/Melt curve (95° C., 15 sec; 64° C., 1 min; 90° C., 30 sec; 60° C. 15 sec). The amplification plots and melt curves were analyzed with the Biorad software. The results for each sample were compared to negative control (ultrapure water) and to the positive control (DNA from Tg embryos).

### Primary Culture of Spinal Cord Motor Neurons

Rat spinal cord motor neurons (MNs) were be cultured as described by Wang et al. 2013 and Boussicault et al., 2020. Briefly, pregnant female rats of 14 days gestation (Sprague Dawley; Taconic) were killed using a deep anesthesia with CO2 chamber and a cervical dislocation. Then, fetuses were removed from the uterus and immediately placed in ice-cold L15 Leibovitz medium with a 2% penicillin (10,000 U/ml) and streptomycin (10 mg/ml) solution (PS) and 1% bovine serum albumin (BSA).

Spinal cords were treated for 20 min at 37° C. with a trypsin-EDTA solution at a final concentration of 0.05% trypsin and 0.02% EDTA. The dissociation was stopped by addition of Dulbecco's modified Eagle's medium (DMEM) with 4.5 g/liter of glucose, containing DNAse I grade II (final concentration 0.5 mg/mL) and 10% fetal calf serum (FCS). Cells were mechanically dissociated by three forced passages through the tip of a 10-ml pipette. Cells were then centrifuged at 515×g for 10 min at 4° C. The supernatant was discarded, and the pellet was resuspended in a defined culture medium consisting of Neurobasal medium with a 2% solution of B27 supplement, 2 mmol/liter of L-glutamine, 2% of PS solution, and 10 ng/ml of brain-derived neurotrophic factor (BDNF). Viable cells were counted in a Neubauer cytometer, using the trypan blue exclusion test. The cells were seeded at a density of 20,000 per well in 96-well plates precoated with poly-L-lysine and will be cultured at 37° C. in an air (95%)—CO2 (5%) incubator. The medium was changed every 2 days. The wells of the first lines and columns were not used for culture (to avoid any edge effect) and were filled with sterile water. The spinal cord motor neurons were injured with glutamate after 13 days of culture.

### Test Compounds and Glutamate Exposure:

Vehicle: Culture medium (0.1% DMSO)
i) Pre-incubation. On day 13 of culture, primary motor neurons were pre-treated with the test compounds for 1 hour.
ii) Injury. On day 13 of culture, and after the 1 hour of pre-incubation, glutamate was added to a final concentration of 5 μM diluted in control medium still in presence of the compounds for 20 min. After 20 min, glutamate was washed out and fresh culture medium was added for an additional 24 hours.

Test compounds were tested on cultures in 96-well plates (6 wells per condition)

### Endpoint Evaluation:

Immunostaining: MAP-2 and TDP43. 24 hours after intoxication, the supernatants were discarded, and cells were fixed by a cold solution of ethanol (95%) and acetic acid (5%) for 5 min at −20° C. After permeabilization with 0.1% of saponin, cells were incubated for 2 hours with: (a) a mouse monoclonal antibody anti microtubule-associated-protein 2 (MAP-2) at dilution of 1/400 in PBS containing 1% fetal calf serum and 0.1% of saponin. This antibody was revealed with Alexa Fluor 488 goat anti-mouse IgG at the dilution 1/400 in PBS containing 1% FCS, 0.1% saponin, for 1 hour at room temperature, and (b) a rabbit polyclonal antibody anti-nuclear TAR DNA-binding protein 43 (TDP-43) at dilution of 1/100 in PBS containing 1% fetal calf serum and 0.1% of saponin. The antibody TDP-43 was revealed with Alexa Fluor 568 goat anti-rabbit at a dilution of 1/400 in PBS containing 1% FCS, 0.1% saponin, for 1 hour at room temperature.

### Automatic Computer Analysis

For each condition, 30 pictures (representative of the all well area) per well were automatically taken using ImageXpress® (Molecular Devices) with 20× magnification, using the same acquisition parameters. From images, analyses were directly and automatically performed by MetaXpress® (Molecular Devices).

The following endpoints were automatically assessed:
i) Analysis of motor neuron survival (number of motor neurons),
ii) Analysis of motor neuron neurite network (neurite length in μm)
iii) Cytoplasmic area of TDP-43 in motor neurons (overlapping between MAP-2 and cytoplasmic TDP-43 in μm²).

Compounds 157, 185 and 196 showed positive effects in this in vitro model of ALS.

Compound 157 had beneficial effects upon the neurite network (a >20% increase in neurite network length compared to the glutamate injury alone case, at compound concentrations of 0.1 μM, 0.5 μM and 1 μM) and fully abolished the abnormal TDP43 accumulation in the cytoplasm normally resulting from glutamate injury (at compound concentrations of 0.1 μM, 0.5 M, 1 μM and 10 μM).

Compound 185 fully abolished the abnormal cytoplasmic TDP-43 accumulation normally resulting from glutamate injury (at compound concentrations of 0.1 μM, 0.5 UM and 1 μM).

Compound 196 improved the neuronal survival (a >10% increase in number of motor neurons compared to the glutamate injury alone case, at compound concentrations of 1 and 10 μM), protected the neurite network (a 16% increase in neurite network length compared to the glutamate injury alone case at a compound concentration of 10 μM) and fully abolished the abnormal TDP43 accumulation in the cytoplasm normally resulting from glutamate injury (at compound concentrations of 0.5 μM, 1 μM and 10 μM).

This model functions as an in vitro model of Amyotrophic lateral sclerosis (ALS). Activity (positive effects) in this model strongly support that compounds of this invention are efficacious in the treatment of Amyotrophic lateral sclerosis and other autophagy-related neurodegenerative diseases or conditions.

### REFERENCES

Boussicault L, Laffaire J, Schmitt P, Rinaudo P, Callizot N, Nabirotchkin S, Hajj R, Cohen D. Combination of acamprosate and baclofen (PXT864) as a potential new therapy for amyotrophic lateral sclerosis. J Neurosci Res. 2020 December; 98 (12): 2435-2450.
Sakka L, Delétage N, Lalloué F, Duval A, Chazal J, Lemaire J J, Meiniel A, Monnerie H, Gobron S. SCO-spondin derived peptide NX210 induces neuroprotection in vitro and promotes fiber regrowth and functional recovery after spinal cord injury. PLOS One. 2014 Mar. 25; 9 (3): e93179.
Vera A, Stanic K, Montecinos H, Torrejón M, Marcellini S, Caprile T. SCO-spondin from embryonic cerebrospinal fluid is required for neurogenesis during early brain development. Front Cell Neurosci. 2013 Jun. 3; 7:80.
Wang W, Li L, Lin W L, Dickson D W, Petrucelli L, Zhang T, Wang X. The ALS disease-associated mutant TDP-43 impairs mitochondrial dynamics and function in motor neurons. Hum Mol Genet. 2013 Dec. 1; 22 (23): 4706-19

### Example 2

### Demonstration of Activity in a Mouse Model of Parkinson's Disease (PD)

Compound 196 (particularly preferred) was selected as example compound of this invention to be evaluated in this model.

This model (run by Neuro-Sys SAS, France) uses C57BL/6JRj mice injured by intra-nigral injections of a solution containing protofibrils of alpha-synuclein (α-syn, precisely quantified by automatic Western Blot) and combined with pharmacological inhibition of GBA by conduritol B epoxide, in aged mice. The model reproduces essential neuropathological features of PD (e.g. loss of dopaminergic neurons, α-syn aggregation, endoplasmic reticulum [ER] stress) and the neuroinflammatory response (microglia activation and release of proinflammatory cytokines) in the nigra striata area. Any substances with positive effects in this model may be useful as a new therapeutic agent for the treatment or prevention of PD.

### Overview of the Study

Animal characteristics:
Type of animal: C57BL/6JRj (Male)
Age: 18 months old
Supplier: Janvier Labs
Animal number: 7 mice per group
Acclimation and clinical signs: Animals arrived on site 7 days (one week) before the experiment to allow optimal acclimation.

### Dosing Outline:

Test compound dose and administration: 30 mg/kg, per os (gavage or drinking water), twice a day
Test compound treatment: The test compound was solubilized (vehicle=water), aliquoted and stored at −20° C. Aliquots were thawed overnight at 4° C. and given twice a day (morning and afternoon). Treatment started on the day of the stereotaxic injections of α-syn preparation (Day 0). Treatment was administrated until the last day of the experiment (Day 28; =day of dissection).
CBE dose and administration: 50 mg/kg, Intraperitoneal (i.p.), twice a day.
CBE treatment: Administration of CBE (vehicle=saline [NaCl 0.9%] started on the day of the stereotaxic injections of α-syn preparation (Day 0). Treatment was administrated until the last day of the experiment (Day 28; =day of dissection),
Untreated control: vehicle (saline; i.p.) administered twice a day,

### Readouts:

i) Body mass.
ii) Dopaminergic Tyrosine hydroxylase (TH)-positive neuron survival in the substantia nigra pars compacta (SNpc), i.e. a measure of the number of TH neurons in the SNpc.
iii) Aggregation of α-syn in TH neurons, in the SNpc.
iv) Activation of Ionized calcium-binding adaptor protein-1 (Iba-1)-positive microglial cells in the SNpc.

### Methods

### Stereotaxic Injections of α-Syn Oligomers

Human α-syn peptide (stock at 69 μM in water at −20° C.) was reconstituted at 50 μM in NaCl, 0.9% (final concentration). All mice in compound treated groups were subjected to surgery and received 2.5 μL of α-syn solution. Mice were anesthetized by isoflurane (4%, for induction), in an induction chamber coupled with a vaporizer and to an oxygen concentrator. Mice were placed on the stereotaxic frame. Anesthesia was maintained by isoflurane (2%) with a face mask coupled to the isoflurane vaporizer and oxygen concentrator machine. The skull was exposed and holes were drilled. The α-syn preparation was bilaterally injected into the SNpc, at the following coordinates: A-P, −0.3 mm; M-L, +0.12 mm; D-V, −0.45 mm. Depth of anesthesia and rectal temperature were verified every 5 minutes. After surgery, mice were allowed to recover before being placed back in the cage.

### Endpoint Evaluation

For overall evaluation and body mass, mice were observed daily, and body mass was monitored prior the morning drug administration.

### Plasma Sampling, Tissue Collection and Immunostaining.

At the end of the experiment (on day 28 post-surgery), mice (n=5/group) were deeply anesthetized, whole blood was collected on heparin tubes by heart puncture, centrifugated at 1500 g, 10 minutes, 4° C. Plasma was collected and snap frozen. Directly after heart puncture, mice were perfused with cold PBS (3 minutes), and cold paraformaldehyde (PFA) 4% in PBS (3 minutes). Immunostaining was performed with the n=5/group. Brains were dissected and further fixed in PFA 4%, overnight at 4° C. After, brains were placed in 30% sucrose in Tris-phosphate saline (TBS) solution at 4° C. Coronal sections, including the SNpc, of 40 μm-thickness were cut using a freezing microtome (4 sections per mouse, each 100 mm apart). For immunostaining, free-floating sections were incubated in TBS with 0.25% bovine serum albumin, 0.3% Triton X-100 and 1% goat serum, for 1 hour at room temperature. This incubation blocked unspecific binding sites and permeabilized the tissues. Four (n=4) brain sections per animal were processed and incubated for 24 hours at 4° C. or 2 hours at room temperature with selected antibodies:
TH: Chicken polyclonal antibody anti-tyrosine hydroxylase (1/1000).
α-syn: Rabbit polyclonal antibody anti-alpha synuclein (1/200).
IBA1: Goat polyclonal antibody anti-IBA1 (1/200)

These antibodies were revealed with Alexa Fluor 488 anti-rabbit IgG, Alexa Fluor 568 anti-chicken IgG, at the dilution 1/500, incubated in TBS with 0.25% donkey serum albumin, 0.3% Triton X-100 and 1% goat serum.

Images were acquired with a confocal laser-scanning microscopy. The following read-outs were investigated:
Number of TH positive cells in the SNpc.
Activation of Iba1-positive microglial cells in the SNpc.
Aggregation of α-syn in TH neurons, in the SNpc.

### Results

Compound 196 did not significantly modify body mass of the animals after 4 weeks of treatment, suggesting the absence of a systemic toxic effect of Compound 196 at the analyzed time points. A >60% increase in the number of dopaminergic TH positive neurons in the SNpc was observed in the compound treated group compared to the α-syn/CBE injured group. In addition, a >15% decrease in the number of Iba1-positive microglial cells in the SNpc was observed in the compound treated group compared to the α-syn/CBE injured group. Taken together, this data indicates that Compound 196 was able to significantly protect dopaminergic TH positive neurons from neurodegeneration in this study.

This model functions as an in vivo model of Parkinson's disease. Activity in this model (positive effects) strongly support that compounds of this invention are efficacious in the treatment of Parkinson's disease and other autophagy-related neurodegenerative diseases or conditions.

### Bioavailability

The compounds according to the invention have good properties for oral dosing and readily cross the blood brain barrier. For example, in CD1 mice, Compound 157 has a maximum concentration in brain plasma of 2153 ng/ml after 1.5 hours at an oral dose of 30 mg/kg with a half-life in the brain of 1.8 hours. It also had a half-life in plasma of 1.8 hours and 81% bioavailability. Compound 185 has a maximum concentration in brain plasma of 3457 ng/ml after 1 hour at an oral dose of 30 mg/kg with a half-life in the brain of 13 hours. It also had a half-life in plasma of 18 hours and 100% bioavailability.

Hence, the compounds are suitable for oral dosing making them advantageous for the treatment of various conditions and in particular for the treatment of neurodegenerative disorders.

1. A pharmaceutical composition comprising one or more of a compound according to Formula (I), 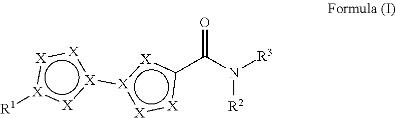
a physiologically acceptable salt of the compound according to Formula (I), a solvate of the compound according to Formula (I), a hydrate of the compound according to Formula (I), an enantiomer of the compound of Formula (I), and a polymorph of the compound according to Formula (I),
wherein
X is independently selected from chemically possible combinations of C, N,
O, and S, and is optionally-substituted with one of —CH₃, —CH₂—CH₃ or COOH;
R¹ is selected from cyclic C₅ or C₆ alkyl, and optionally comprising at least one of chemically possible N, O, and S, and optionally one of mono- or bi-substituted with one or more of —CH3, —NH₂, —COOH, C₁ to C₄ alkoxy, halo, trifluoromethyl, and trifluoromethoxy;
R² is selected from H, CH₃, straight or branched C₂ to C₆ alkyl, and optionally comprising at least one of chemically possible N, O, and S, and optionally substituted with one or —CH₃, —NH₂, —OH, —COOH, cyclopentyl, cyclohexyl, bicyclo[2.2.1]heptane, and bicyclo[3.1.1]heptane, bicyclo[2.2.2]octane, optionally comprising at least one of chemically possible N, O, and S, and optionally substituted with one of —CH₃, —CH₂—CH₃, —OH, -isopropyl, —COOH, —COOCH₃, —CH₂-cyclohexyl, and NH₂;
R³ is selected from H, straight or branched C₁ to C₆ alkyl, optionally substituted with —CH₃, —OH, —CH₂—CH₃, —NH₂, —CH₂—NH₂, —CH₂—NH—CH₃, —COOH, and optionally comprising chemically possible at least one of one or more N, O, and S;
the C₁ to C₄ alkoxy, optionally comprising at least one of chemically possible one or more N, O, and S, and optionally mono- or bi substituted with, one or more of —NH₂, —OH, and —COOH;
cyclopropyl, cyclobutyl, optionally comprising chemically possible at least one of one or more N, O, and S, and optionally mono- or bi 34 substituted with one or more of —CH₃, —CH₂—CH₃, —NH₂, —CH₂—NH₂, —CH₂—NH—CH₃, —OH, —COOH;
with the provisio that R² and R³ are not both H or CH₃,
R² and R³ may be connected to form a 4-membered, 5-membered, 6-membered, 7-membered ring, a bicyclic ring structure of at least one of fused cyclopentyl and cyclohexyl, bicyclo[3.1.1]heptane, optionally comprising chemically possible one or more N, O, and S, and optionally being mono- or bi-substituted with at least one of straight and branched C₁ to C₆ alkyl, optionally substituted with one or more of —CH₃, —OH, —CH₂—CH₃, —NH₂, —NH—CH₃, —CH₂—NH₂, —CH₂—NH—CH₃, —OH, —COOH, —COOCH₃, ═O, isopropyl, sulfonamide, and optionally including chemically possible one or more of N, O, and S;
with the provisio that either R² or R³ or the combination thereof include at least one saturated/fully substituted nitrogen.

2. A pharmaceutical composition according to claim 1, wherein the compound according to Formula (I) is also according to Formula (II), 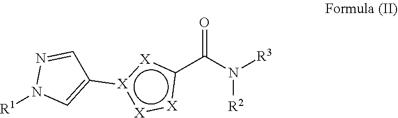
wherein
X, R¹, R² and R³ are as above.

3. A pharmaceutical composition according to claim 1, wherein the compound according to Formula (I) is also according to the Formula (III), 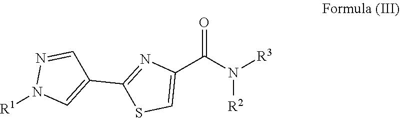
wherein
R¹, R² and R³ are as above.

4. A pharmaceutical composition according to claim 1, wherein
R¹ is selected from functional groups 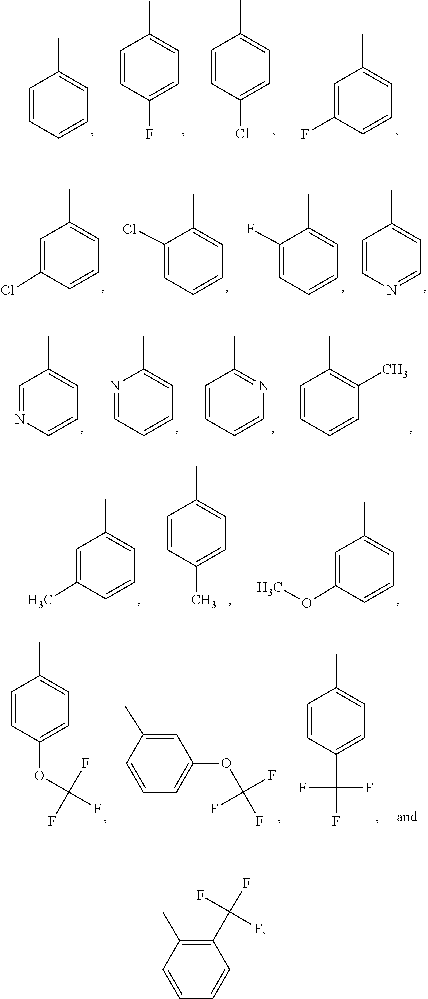
physiologically acceptable salts of the groups, solvates of the groups, hydrates of the groups, enantiomers of the groups, and polymorphs of the groups.

5. A pharmaceutical composition according to claim 1, wherein R² is selected from functional groups H, —CH₃, cyclopentyl, cyclohexyl, 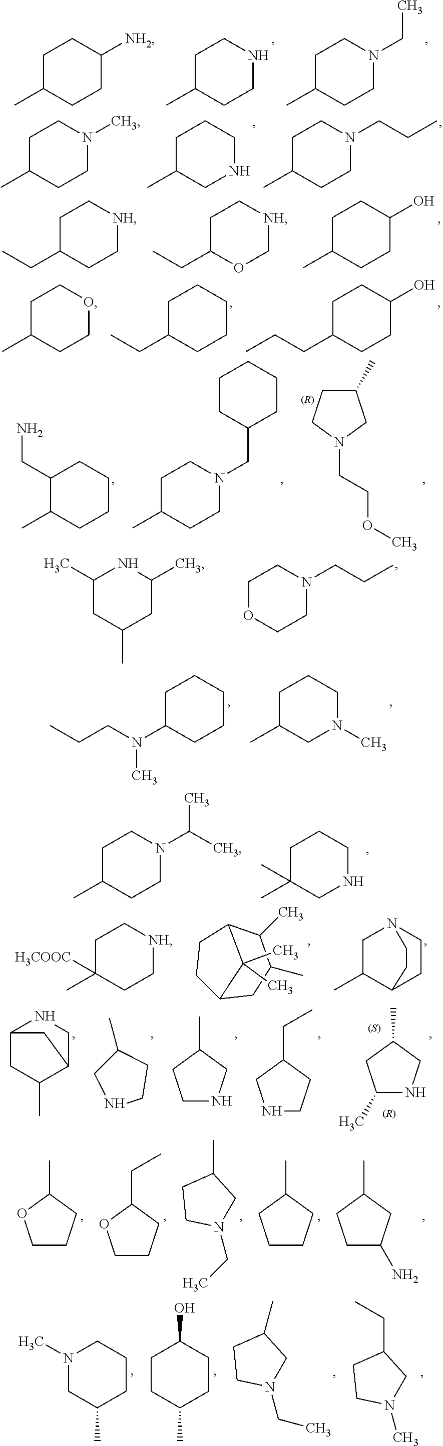 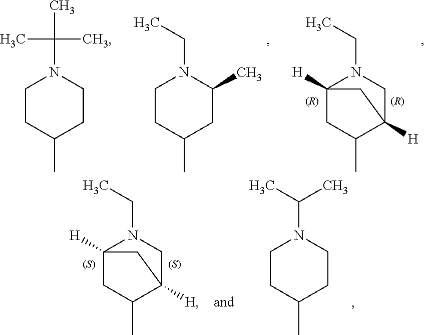
R³ is selected from further functional groups H, —CH₃, —CH₂—CH₃, —CH₂—CH₂—CH₃; propyl, isopropyl, —CH₂—CH₂—OH, —CH₂—CH₂—NH₂, —CH₂—CH₂—CH₂—NH₂, —CH₂—CH₂—CH₂—CH₂—CH₂—NH₂, —CH₂—CH₂—O—CH₂—CH₂—NH₂, —CH₂—CH(CH₃)₂, —CH₂—CH(NH₂)—CH₃, —CH(—CH₃)—CH₂—NH₂, —CH₂—CH₂—N(CH₃)₂, 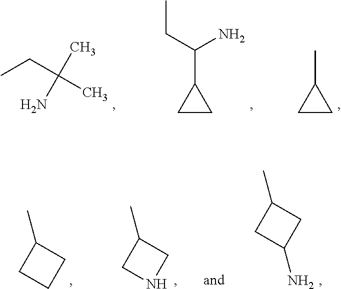
and R² and R³ may be connected to form a ring selected from still further functional groups 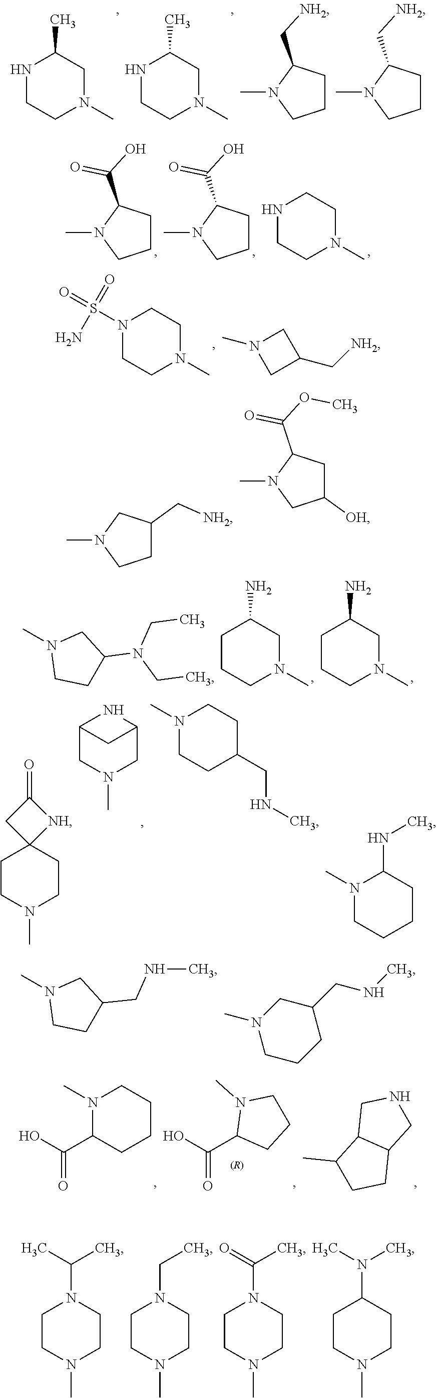 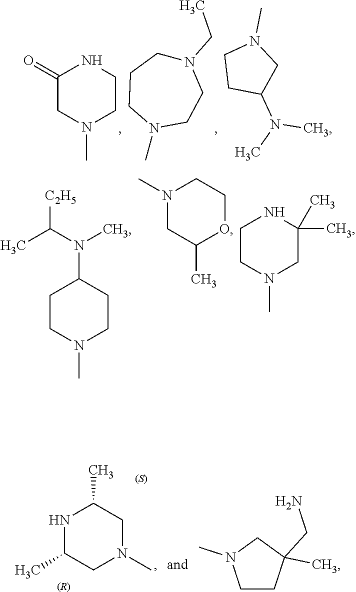
physiologically acceptable salts of the still further functional groups, solvates of the still further functional groups, hydrates of the still further functional groups, enantiomers of the still further functional groups, and polymorphs of the still further functional groups.

6. A pharmaceutical composition according to claim 1, wherein the compound according to Formula (I) is also according to the Formula (IV), 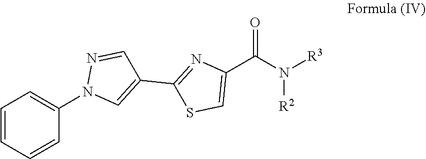
wherein R² is selected from functional groups 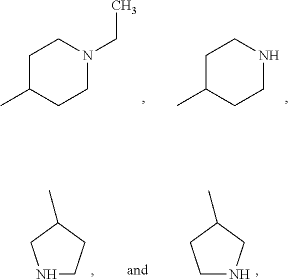
one of R³ is selected from further functional groups H, CH₃, —CH₂—CH₂—CH₃, and isopropyl, and
R² and R³ form a ring selected from still further functional groups 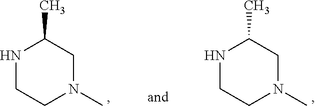
physiologically acceptable salts of the still further functional groups, solvates of the still further functional groups, hydrates of the still further functional groups, enantiomers of the still further functional groups, and polymorphs of the still further functional groups.

7. A pharmaceutical composition according to claim 1, wherein at least one of R² and R³ a combination thereof together comprise at least two nitrogens selected from the group of saturated nitrogens and at least partially unsaturated nitrogens that are protected by at least one chemical group in an ortho-position thereof.

8. A pharmaceutical composition comprising one or more of a physiologically acceptable salt of a compound, a solvate of the compound, a hydrate of the compound, an enantiomer of the compound, and a polymorph of the compound, wherein the compound is selected from the following:
N-(1-ethylpiperidin-4-yl)-N-methyl-5-(1-phenyl-1H-pyrazol-4-yl)-1H-pyrrole-2-carboxamide (Formula 1);
(3S)-3-methyl-1-[4-(5-phenyl-1,3,4-thiadiazol-2-yl)thiophene-2-carbonyl]piperazine (Formula 2);
(3S)-3-methyl-1-[4-(1-phenyl-1H-pyrazol-4-yl)-1H-pyrrole-2-carbonyl]piperazine (Formula 3);
(3S)-3-methyl-1-[5-(1-phenyl-1H-pyrazol-4-yl)thiophene-3-carbonyl]piperazine (Formula 4);
(3S)-3-methyl-1-[2-(5-phenylthiophen-2-yl)-1,3-thiazole-4-carbonyl]piperazine (Formula 7);
N-methyl-N-(oxan-4-yl)-5-(1-phenyl-1H-pyrazol-4-yl) furan-2-carboxamide (Formula 10);
(3S)-3-methyl-1-[2-(2-phenyl-1,3-oxazol-5-yl)-1,3-thiazole-4-carbonyl]piperazine (Formula 13);
N-methyl-N-(oxan-4-yl)-4-(1-phenyl-1H-pyrazol-4-yl)thiophene-2-carboxamide (Formula 15);
N-methyl-N-(oxan-4-yl)-5-(1-phenyl-1H-pyrazol-4-yl)-1H-pyrrole-2-carboxamide (Formula 19);
(3S)-3-methyl-1-(2-{1-[3-(trifluoromethoxy)phenyl]-1H-pyrazol-4-yl}-1,3-thiazole-4-carbonyl) piperazine (Formula 24);
(3S)-3-methyl-1-{2-[1-(3-methylphenyl)-1H-pyrazol-4-yl]-1,3-thiazole-4-carbonyl}piperazine (Formula 27);
(3S)-1-{2-[1-(3-chlorophenyl)-1H-pyrazol-4-yl]-1,3-thiazole-4-carbonyl}-3-methylpiperazine (Formula 30);
(3S)-3-methyl-1-{2-[1-(pyridin-2-yl)-1H-pyrazol-4-yl]-1,3-thiazole-4-carbonyl}piperazine (Formula 33);
(3S)-1-{2-[1-(4-fluorophenyl)-1H-pyrazol-4 yl]-1,3-thiazole-4-carbonyl}-3-methylpiperazine (Formula 34);
(3S)-3-methyl-1-(2-{1-[4-(trifluoromethoxy)phenyl]-1H-pyrazol-4-yl}-1,3-thiazole-4-carbonyl) piperazine (Formula 37);
(3S)-3-methyl-1-(2-{1-[3-(trifluoromethyl)phenyl]-1H-pyrazol-4-yl}-1,3-thiazole-4-carbonyl) piperazine (Formula 38);
(3S)-1-{2-[1-(4-chlorophenyl)-1H-pyrazol-4-yl]-1,3-thiazole-4-carbonyl}-3-methylpiperazine (Formula 39);
(2R)-1-[2-(1-phenyl-1H-pyrazol-4-yl)-1,3-thiazole-4 carbonyl]pyrrolidine-2-carboxylic acid (Formula 55);
(2S)-1-[2-(1-phenyl-1H-pyrazol-4-yl)-1,3-thiazole-4 carbonyl]pyrrolidine-2-carboxylic acid (Formula 60);
(3S)-1-{2-[1-(3-methoxyphenyl)-1H-pyrazol-4-yl]-1,3-thiazole-4 carbonyl}-3-methylpiperazine (Formula 62);
(3S)-3-methyl-1-{2-[1-(4-methylphenyl)-1H-pyrazol-4-yl]-1,3-thiazole-4-carbonyl}piperazine (Formula 63);
(3S)-3-methyl-1-[5-(1-phenyl-1H-pyrazol-4-yl)-1H-pyrrole-2-carbonyl]piperazine (Formula 67);
(3S)-3-methyl-1-(2-{1-[2-(trifluoromethyl)phenyl]-1H-pyrazol-4-yl}-1,3-thiazole-4-carbonyl) piperazine (Formula 72);
(3S)-3-methyl-1-(2-{1-[4-(trifluoromethyl)phenyl]-1H-pyrazol-4-yl}-1,3-thiazole-4-carbonyl) piperazine (Formula 75);
(3S)-1-{2-[1-(2-chlorophenyl)-1H-pyrazol-4-yl]-1,3-thiazole-4-carbonyl}-3-methylpiperazine (Formula 79);
(3S)-3-methyl-1-{2-[1-(2-methylphenyl)-1H-pyrazol-4-yl]-1,3-thiazole-4-carbonyl}piperazine (Formula 85);
(3S)-1-{2-[1-(2-fluorophenyl)-1H-pyrazol-4 yl]-1,3-thiazole-4-carbonyl}-3-methylpiperazine (Formula 96);
(3S)-3-methyl-1-[5-(1-phenyl-1H-pyrazol-4-yl) furan-2-carbonyl]piperazine (Formula 102);
N-(1-ethylpiperidin-4-yl)-4-(1-phenyl-1H-pyrazol-4-yl)thiophene-2-carboxamide (Formula 103);
4-[2-(1-phenyl-1H-pyrazol-4-yl)-1,3-thiazole-4-carbonyl]piperazine-1-sulfonamide (Formula 105);
N-(1-ethylpiperidin-4-yl)-5-(1-phenyl-1H-pyrazol-4-yl) furan-2-carboxamide (Formula 109);
(3S)-3-methyl-1-[4-(1-phenyl-1H-pyrazol-4-yl)thiophene-2-carbonyl]piperazine (Formula 113);
(3R)-3-methyl-1-[2-(1-phenyl-1H-pyrazol-4-yl)-1,3-thiazole-4-carbonyl]piperazine (Formula 118);
Methyl 4-hydroxy-1-[2-(1-phenyl-1H-pyrazol-4-yl)-1,3-thiazole-4-carbonyl]pyrrolidine-2-carboxylate (Formula 125);
N,N-diethyl-1-[2-(1-phenyl-1H-pyrazol-4-yl)-1,3-thiazole-4-carbonyl]pyrrolidin-3-amine (Formula 134);
N-(1-methylpiperidin-4-yl)-2-(1-phenyl-1H-pyrazol-4-yl)-1,3-thiazole-4-carboxamide (Formula 138);
1-[2-(1-phenyl-1H-pyrazol-4-yl)-1,3-thiazole-4-carbonyl]pyrrolidine-2-carboxylic acid (Formula 144);
2-(1-phenyl-1H-pyrazol-4-yl)-N-[(1r,4r)-4-hydroxycyclohexyl]-1,3-thiazole-4-carboxamide (Formula 147);
2-(1-phenyl-1H-pyrazol-4-yl)-N-{2,6,6-trimethylbicyclo[3.1.1]hept an-3-yl}-1,3-thiazole-4-carboxamide (Formula 148);
N-cyclohexyl-2-(1-phenyl-1H-pyrazol-4-yl)-N-propyl-1,3-thiazole-4-carboxamide (Formula 151);
N-cyclohexyl-N-[2-(dimethylamino)ethyl]-2-(1-phenyl-1H-pyrazol-4-yl)-1,3-thiazole-4-carboxamide (Formula 155);
(3S)-3-methyl-1-[2-(1-phenyl-1H-pyrazol-4-yl)-1,3-thiazole-4-carbonyl]piperazine (Formula 157);
N-methyl-1-[2-(1-phenyl-1H-pyrazol-4-yl)-1,3-thiazole-4-carbonyl]-N-(propan-2-yl) piperidin-4-amine (Formula 164);
7-[2-(1-phenyl-1H-pyrazol-4-yl)-1,3-thiazole-4-carbonyl]-1,7-diazaspiro[3.5]nonan-2-one (Formula 166);
2-methyl-4-[2-(1-phenyl-1H-pyrazol-4-yl)-1,3-thiazole-4-carbonyl]morpholine (Formula 168);
N-methyl-N-[(oxolan-2-yl)methyl]-2-(1-phenyl-1H-pyrazol-4-yl)-1,3-thiazole-4 carboxamide (Formula 169);
N-methyl-N-(oxan-4-yl)-2-(1-phenyl-1H-pyrazol-4-yl)-1,3-thiazole-4-carboxamide (Formula 172);
N-ethyl-N-methyl-2-(1-phenyl-1H-pyrazol-4-yl)-1,3-thiazole-4-carboxamide (Formula 173);
N-(1-benzylpyrrolidin-3-yl)-2-(1-phenyl-1H-pyrazol-4-yl)-1,3-thiazole-4-carboxamide (Formula 174);
N-[2-(4-hydroxyphenyl)ethyl]-2-(1-phenyl-1H-pyrazol-4-yl)-1,3-thiazole-4-carboxamide (Formula 176);
N-(1-benzylpiperidin-4-yl)-2-(1-phenyl-1H-pyrazol-4-yl)-1,3-thiazole-4-carboxamide (Formula 182);
N-(1-ethylpiperidin-4-yl)-2-(1-phenyl-1H-pyrazol-4-yl)-1,3-thiazole-4-carboxamide (Formula 185);
N-{1-azabicyclo[2.2.2]octan-3-yl}-N-methyl-2-(1-phenyl-1H-pyrazol-4-yl)-1,3-thiazole-4-carboxamide (Formula 186);
N,N-dimethyl-1-[2-(1-phenyl-1H-pyrazol-4-yl)-1,3-thiazole-4-carbonyl]pyrrolidin-3-amine (Formula 187);
N-(1-ethylpyrrolidin-3-yl)-N-methyl-2-(1-phenyl-1H-pyrazol-4-yl)-1,3-thiazole-4carboxamide (Formula 188);
N-methyl-N-[(1-methylpyrrolidin-3-yl)methyl]-2-(1-phenyl-1H-pyrazol-4-yl)-1,3-thiazole-4carboxamide (Formula 189);
N-{2-[cyclohexyl(methyl)amino]ethyl}-N-methyl-2-(1-phenyl-1H-pyrazol-4-yl)-1,3-thiazole-4-carboxamide (Formula 190);
N-cyclopentyl-N-(2-methylpropyl)-2-(1-phenyl-1H-pyrazol-4-yl)-1,3-thiazole-4-carboxamide (Formula 191);
N-cyclopentyl-N-ethyl-2-(1-phenyl-1H-pyrazol-4-yl)-1,3-thiazole-4-carboxamide (Formula 192);
1-ethyl-4-[2-(1-phenyl-1H-pyrazol-4-yl)-1,3-thiazole-4 carbonyl]-1,4-diazepane (Formula 193);
N-cyclopentyl-N-cyclopropyl-2-(1-phenyl-1H-pyrazol-4-yl)-1,3-thiazole-4-carboxamide (Formula 194);
N-methyl-2-(1-phenyl-1H-pyrazol-4-yl)-N-[1-(propan-2-yl) piperidin-4-yl]-1,3-thiazole-4-carboxamide (Formula 195);
N-(1-ethylpiperidin-4-yl)-N-methyl-2-(1-phenyl-1H-pyrazol-4-yl)-1,3-thiazole-4 carboxamide (Formula 196);
N,N-dimethyl-1-[2-(1-phenyl-1H-pyrazol-4-yl)-1,3-thiazole-4-carbonyl]piperidin-4-amine (Formula 197);
1-{4-[2-(1-phenyl-1H-pyrazol-4-yl)-1,3-thiazole-4carbonyl]piperazin-1-yl}ethan-1-one (Formula 198);
1-ethyl-4-[2-(1-phenyl-1H-pyrazol-4-yl)-1,3-thiazole-4 carbonyl]piperazine (Formula 199);
4-[2-(1-phenyl-1H-pyrazol-4-yl)-1,3-thiazole-4-carbonyl]piperazin-2-one (Formula 200);
N-cyclohexyl-N-methyl-2-(1-phenyl-1H-pyrazol-4-yl)-1,3-thiazole-4-carboxamide (Formula 201);
2-(1-phenyl-1H-pyrazol-4-yl)-N-[2-(piperidin-1-yl)ethyl]-1,3-thiazole-4-carboxamide (Formula 202);
N-(cyclohexylmethyl)-2-(1-phenyl-1H-pyrazol-4-yl)-1,3-thiazole-4-carboxamide (Formula 203);
N-[2-(morpholin-4-yl)ethyl]-2-(1-phenyl-1H-pyrazol-4-yl)-1,3-thiazole-4 carboxamide (Formula 204);
N-[(3S)-1-methylpiperidin-3-yl]-2-(1-phenyl-1H-pyrazol-4-yl)-1,3-thiazole-4 carboxamide (Formula 208);
1-[2-(1-phenyl-1H-pyrazol-4-yl)-1,3-thiazole-4-carbonyl]-4-(propan-2-yl) piperazine (Formula 209);
2-{4-[2-(1-phenyl-1H-pyrazol-4-yl)-1,3-thiazole-4 carbonyl]piperazin-1-yl}-N-(propan-2-yl) acetamide (Formula 210);
(3S)-1-{2-[1-(3-fluorophenyl)-1H-pyrazol-4-yl]-1,3-thiazole-4-carbonyl}-3-methylpiperazine (Formula 211);
(3S)-1-{2-[1-(4-fluorophenyl)-1H-pyrazol-4-yl]-1,3-thiazole-4-carbonyl}-3-methylpiperazine (Formula 212);
N-(1-ethylpiperidin-4-yl)-2-[1-(4-fluorophenyl)-1H-pyrazol-4-yl]-1,3-thiazole-4-carboxamide (Formula 213);
N-(1-ethylpiperidin-4-yl)-5-(1-phenyl-1H-pyrazol-4-yl)-1H-pyrrole-2-carboxamide (Formula 214);
2-(1-phenyl-1H-pyrazol-4-yl)-N-[1-(propan-2-yl) piperidin-4-yl]-1,3-thiazole-4-carboxamide (Formula 215);
4-(5-{1-[2-(trifluoromethyl)phenyl]-1H-pyrazol-4-yl}-1H-pyrrole-2-carbonyl) piperazine-1-sulfonamide (Formula 216);
N-(1-ethylpiperidin-4-yl)-N-methyl-2-{1-[2-(trifluoromethyl)phenyl]-1H-pyrazol-4-yl}-1,3-thiazole-4-carboxamide (Formula 217);
N-(1-ethylpiperidin-4-yl)-2-(1-phenyl-1H-pyrazol-4-yl)-N-(propan-2-yl)-1,3-thiazole-4-carboxamide (Formula 218); N-ethyl-N-(1-ethylpiperidin-4-yl)-2-(1-phenyl-1H-pyrazol-4-yl)-1,3-thiazole-4-carboxamide (Formula 219);
(3S)-3-methyl-1-{2-[1-(pyridin-2-yl)-1H-pyrazol-4-yl]-1,3-thiazole-4-carbonyl}piperazine (Formula 220);
N-(1-ethylpiperidin-4-yl)-N-methyl-2-[1-(pyridin-2-yl)-1H-pyrazol-4-yl]-1,3-thiazole-4-carboxamide (Formula 221);
N-(1-ethylpiperidin-4-yl)-2-[1-(pyridin-2-yl)-1H-pyrazol-4-yl]-1,3-thiazole-4-carboxamide (Formula 222);
(3S)-3-methyl-1-{2-[1-(pyridin-3-yl)-1H-pyrazol-4-yl]-1,3-thiazole-4-carbonyl}piperazine (Formula 223);
N-(1-ethylpiperidin-4-yl)-N-methyl-2-[1-(pyridin-3-yl)-1H-pyrazol-4-yl]-1,3-thiazole-4-carboxamide (Formula 224);
N-(1-ethylpiperidin-4-yl)-2-[1-(pyridin-3-yl)-1H-pyrazol-4-yl]-1,3-thiazole-4-carboxamide (Formula 225);
(3S)-3-methyl-1-{2-[1-(pyridin-4-yl)-1H-pyrazol-4-yl]-1,3-thiazole-4-carbonyl}piperazine (Formula 226);
N-(1-ethylpiperidin-4-yl)-N-methyl-2-[1-(pyridin-4-yl)-1H-pyrazol-4-yl]-1,3-thiazole-4-carboxamide (Formula 227);
N-(1-ethylpiperidin-4-yl)-2-[1-(pyridin-4-yl)-1H-pyrazol-4-yl]-1,3-thiazole-4-carboxamide (Formula 228);
3-(4-{4-[(3S)-3-methylpiperazine-1-carbonyl]-1,3-thiazol-2-yl}-1H-pyrazol-1-yl)pyridazine (Formula 229);
N-(1-ethylpiperidin-4-yl)-N-methyl-2-[1-(pyridazin-3-yl)-1H-pyrazol-4-yl]-1,3-thiazole-4-carboxamide (Formula 230);
N-(1-ethylpiperidin-4-yl)-2-[1-(pyridazin-3-yl)-1H-pyrazol-4-yl]-1,3-thiazole-4-carboxamide (Formula 231);
2-(4-{4-[(3S)-3-methylpiperazine-1-carbonyl]-1,3-thiazol-2-yl}-1H-pyrazol-1-yl) pyrazine (Formula 232);
N-(1-ethylpiperidin-4-yl)-N-methyl-2-[1-(pyrazin-2-yl)-1H-pyrazol-4-yl]-1,3-thiazole-4-carboxamide (Formula 233);
N-(1-ethylpiperidin-4-yl)-2-[1-(pyrazin-2-yl)-1H-pyrazol-4-yl]-1,3-thiazole-4-carboxamide (Formula 234);
3,3-dimethyl-1-[2-(1-phenyl-1H-pyrazol-4-yl)-1,3-thiazole-4-carbonyl]piperazine (Formula 235);
1-ethyl-4-[2-(1-phenyl-1H-pyrazol-4-yl)-1,3-thiazole-4-carbonyl]piperazine (Formula 236);
(3R,5S)-3,5-dimethyl-1-[2-(1-phenyl-1H-pyrazol-4-yl)-1,3-thiazole-4-carbonyl]piperazine (Formula 237);
(3S)-3-methyl-1-[2-(2-phenyl-2H-1,2,3-triazol-4-yl)-1,3-thiazole-4-carbonyl]piperazine (Formula 238);
N-(1-tert-butylpiperidin-4-yl)-N-methyl-2-(1-phenyl-1H-pyrazol-4-yl)-1,3-thiazole-4-carboxamide (Formula 239);
N-methyl-2-(1-phenyl-1H-pyrazol-4-yl)-N-[1-(propan-2-yl) piperidin-4-yl]-1,3-thiazole-4-carboxamide (Formula 240);
N-methyl-N-[(2S)-2-methylpiperidin-4-yl]-2-(1-phenyl-1H-pyrazol-4-yl)-1,3-thiazole-4-carboxamide (Formula 241);
N-[(2S)-1-ethyl-2-methylpiperidin-4-yl]-N-methyl-2-(1-phenyl-1H-pyrazol-4-yl)-1,3-thiazole-4-carboxamide (Formula 242);
N-[(2S)-1-ethyl-2-methylpiperidin-4-yl]-2-(1-phenyl-1H-pyrazol-4-yl)-1,3-thiazole-4-carboxamide (Formula 243);
N-[(1R,4R)-2-ethyl-2-azabicyclo[2.2.1]heptan-5-yl]-2-(1-phenyl-1H-pyrazol-4-yl)-1,3-thiazole-4-carboxamide (Formula 244);
N-[(1S,4S)-2-ethyl-2-azabicyclo[2.2.1]heptan-5-yl]-N-methyl-2-(1-phenyl-1H-pyrazol-4-yl)-1,3-thiazole-4-carboxamide (Formula 245);
N-[(1S,4S)-2-ethyl-2-azabicyclo[2.2.1]heptan-5-yl]-2-(1-phenyl-1H-pyrazol-4-yl)-1,3-thiazole-4-carboxamide (Formula 246);
N-(1-ethyl-1H-pyrrol-3-yl)-N-methyl-2-(1-phenyl-1H-pyrazol-4-yl)-1,3-thiazole-4-carboxamide (Formula 247);
N-(1-tert-butylpiperidin-4-yl)-2-(1-phenyl-1H-pyrazol-4-yl)-1,3-thiazole-4-carboxamide (Formula 248);
2-(4-{4-[(3S)-3-methylpiperazine-1-carbonyl]-1,3-thiazol-2-yl}-1H-pyrazol-1-yl)pyrimidine (Formula 249);
N-(1-ethylpiperidin-4-yl)-N-methyl-2-[1-(pyrimidin-2-yl)-1H-pyrazol-4-yl]-1,3-thiazole-4-carboxamide (Formula 250);
N-(1-ethylpiperidin-4-yl)-2-[1-(pyrimidin-2-yl)-1H-pyrazol-4-yl]-1,3-thiazole-4-carboxamide (Formula 251);
5-(4-{4-[(3S)-3-methylpiperazine-1-carbonyl]-1,3-thiazol-2-yl}-1H-pyrazol-1-yl)pyrimidine (Formula 252);
N-(1-ethylpiperidin-4-yl)-N-methyl-2-[1-(pyrimidin-5-yl)-1H-pyrazol-4-yl]-1,3-thiazole-4-carboxamide (Formula 253);
N-(1-ethylpiperidin-4-yl)-2-[1-(pyrimidin-5-yl)-1H-pyrazol-4-yl]-1,3-thiazole-4-carboxamide (Formula 254);
(3S)-3-methyl-1-[4-(1-phenyl-1H-pyrazol-4-yl)-1H-pyrrole-2-carbonyl]piperazine (Formula 255);
N-(1-ethylpiperidin-4-yl)-N-methyl-4-(1-phenyl-1H-pyrazol-4-yl)-1H-pyrrole-2-carboxamide (Formula 256);
N-(1-ethylpiperidin-4-yl)-4-(1-phenyl-1H-pyrazol-4-yl)-1H-pyrrole-2-carboxamide (Formula 257);
N-(1-ethylpiperidin-4-yl)-N-methyl-4-(5-phenyl-1,3,4-thiadiazol-2-yl)thiophene-2-carboxamide (Formula 258);
N-(1-ethylpiperidin-4-yl)-4-(5-phenyl-1,3,4-thiadiazol-2-yl)thiophene-2-carboxamide (Formula 259);
(3S)-3-methyl-1-[2-(1-phenyl-1H-1,2,4-triazol-3-yl)-1,3-thiazole-4-carbonyl]piperazine (Formula 260);
N-(1-ethylpiperidin-4-yl)-N-methyl-2-(1-phenyl-1H-1,2,4-triazol-3-yl)-1,3-thiazole-4-carboxamide (Formula 261);
N-(1-ethylpiperidin-4-yl)-2-(1-phenyl-1H-1,2,4-triazol-3-yl)-1,3-thiazole-4-carboxamide (Formula 262);
(3S)-3-methyl-1-[2-(1-phenyl-1H-pyrazol-4-yl)-1,3-oxazole-4-carbonyl]piperazine (Formula 263);
N-(1-ethylpiperidin-4-yl)-N-methyl-2-(1-phenyl-1H-pyrazol-4-yl)-1,3-oxazole-4-carboxamide (Formula 264);
N-(1-ethylpiperidin-4-yl)-2-(1-phenyl-1H-pyrazol-4-yl)-1,3-oxazole-4-carboxamide (Formula 265);
(3S)-1-{2-[1-(4-cyclopropylphenyl)-1H-pyrazol-4-yl]-1,3-thiazole-4-carbonyl}-3-methylpiperazine (Formula 266);
(3S)-1-{2-[1-(4-cyclopropylphenyl)-1H-pyrazol-4-yl]-1,3-thiazole-4-carbonyl}-3-methylpiperazine (Formula 267);
2-[1-(4-cyclopropylphenyl)-1H-pyrazol-4-yl]-N-(1-ethylpiperidin-4-yl)-1,3-thiazole-4-carboxamide (Formula 268);
2-[1-(4-cyclopropylphenyl)-1H-pyrazol-4-yl]-N-(1-ethylpiperidin-4-yl)-N-methyl-1,3-thiazole-4-carboxamide (Formula 269);
(3S)-1-{2-[1-(4-tert-butylphenyl)-1H-pyrazol-4-yl]-1,3-thiazole-4-carbonyl}-3-methylpiperazine (Formula 270);
2-[1-(4-tert-butylphenyl)-1H-pyrazol-4-yl]-N-(1-ethylpiperidin-4-yl)-1,3-thiazole-4-carboxamide (Formula 271);
2-[1-(4-tert-butylphenyl)-1H-pyrazol-4-yl]-N-(1-ethylpiperidin-4-yl)-N-methyl-1,3-thiazole-4-carboxamide (Formula 272);
(3S)-3-methyl-1-(2-{1-[4-(propan-2-yl)phenyl]-1H-pyrazol-4-yl}-1,3-thiazole-4-carbonyl) piperazine (Formula 273);
N-(1-ethylpiperidin-4-yl)-2-{1-[4-(propan-2-yl)phenyl]-1H-pyrazol-4-yl}-1,3-thiazole-4-carboxamide (Formula 274);
N-(1-ethylpiperidin-4-yl)-N-methyl-2-{1-[4-(propan-2-yl)phenyl]-1H-pyrazol-4-yl}-1,3-thiazole-4-carboxamide (Formula 275);
(3S)-3-methyl-1-[2-(1-phenyl-1H-pyrazol-4-yl)-1H-imidazole-4-carbonyl]piperazine (Formula 276);
N-(1-ethylpiperidin-4-yl)-N-methyl-2-(1-phenyl-1H-pyrazol-4-yl)-1H-imidazole-4-carboxamide (Formula 277);
N-(1-ethylpiperidin-4-yl)-2-(1-phenyl-1H-pyrazol-4-yl)-1H-imidazole-4-carboxamide (Formula 278);
(3S)-3-methyl-1-[5-(1-phenyl-1H-pyrazol-4-yl)-1H-imidazole-2-carbonyl]piperazine (Formula 279);
N-(1-ethylpiperidin-4-yl)-N-methyl-5-(1-phenyl-1H-pyrazol-4-yl)-1H-imidazole-2-carboxamide (Formula 280);
N-(1-ethylpiperidin-4-yl)-5-(1-phenyl-1H-pyrazol-4-yl)-1H-imidazole-2-carboxamide (Formula 281);
N-(1-ethylpiperidin-4-yl)-N-(propan-2-yl)-2-[1-(pyridin-2-yl)-1H-pyrazol-4-yl]-1,3-thiazole-4-carboxamide (Formula 282);
N-ethyl-N-(1-ethylpiperidin-4-yl)-2-[1-(pyridin-2-yl)-1H-pyrazol-4-yl]-1,3-thiazole-4-carboxamide (Formula 283);
N-(1-ethylpiperidin-4-yl)-N-methyl-2-[1-(pyridin-2-yl)-1H-pyrazol-4-yl]-1H-imidazole-4-carboxamide (Formula 284);
N-ethyl-N-(1-ethylpiperidin-4-yl)-2-[1-(pyridin-2-yl)-1H-pyrazol-4-yl]-1H-imidazole-4-carboxamide (Formula 285);
N-(1-ethylpiperidin-4-yl)-N-methyl-2-[1-(pyridin-2-yl)-1H-pyrazol-4-yl]-1,3-oxazole-4-carboxamide (Formula 286);
N-ethyl-N-(1-ethylpiperidin-4-yl)-2-[1-(pyridin-2-yl)-1H-pyrazol-4-yl]-1,3-oxazole-4-carboxamide (Formula 287);
N-(1-ethylpiperidin-4-yl)-N-methyl-2-{1-[2-(trifluoromethoxy)phenyl]-1H-pyrazol-4-yl}-1,3-thiazole-4-carboxamide (Formula 288);
(3S)-3-methyl-1-(2-{1-[2-(trifluoromethoxy)phenyl]-1H-pyrazol-4-yl}-1,3-thiazole-4-carbonyl) piperazine (Formula 289);
N-(1-ethylpiperidin-4-yl)-2-{1-[2-(trifluoromethoxy)phenyl]-1H-pyrazol-4-yl}-1,3-thiazole-4-carboxamide (Formula 290);
N-(1-ethylpiperidin-4-yl)-N-methyl-2-{1-[2-(trifluoromethoxy)phenyl]-1H-pyrazol-4-yl}-1,3-oxazole-4-carboxamide (Formula 291);
(3S)-3-methyl-1-(2-{1-[2-(trifluoromethoxy)phenyl]-1H-pyrazol-4-yl}-1,3-oxazole-4-carbonyl) piperazine (Formula 292); 316 N-(1-ethylpiperidin-4-yl)-2-{1-[2-(trifluoromethoxy)phenyl]-1H-pyrazol-4-yl}-1,3-oxazole-4-carboxamide (Formula 293);
N-(1-methylpiperidin-4-yl)-2-(1-phenyl-1H-pyrazol-4-yl)-N-(propan-2-yl)-1,3-thiazole-4-carboxamide (Formula 294);
2-(1-phenyl-1H-pyrazol-4-yl)-N-(propan-2-yl)-N-[1-(propan-2-yl) piperidin-4-yl]-1,3-thiazole-4-carboxamide (Formula 295);
N-(1-tert-butylpiperidin-4-yl)-2-(1-phenyl-1H-pyrazol-4-yl)-N-(propan-2-yl)-1,3-thiazole-4-carboxamide (Formula 296);
N-(2-methylpiperidin-4-yl)-2-(1-phenyl-1H-pyrazol-4-yl)-N-(propan-2-yl)-1,3-thiazole-4-carboxamide (Formula 297);
N-(2,6-dimethylpiperidin-4-yl)-2-(1-phenyl-1H-pyrazol-4-yl)-N-(propan-2-yl)-1,3-thiazole-4-carboxamide (Formula 298);
N-[(3S,5R)-5-methylpyrrolidin-3-yl]-2-(1-phenyl-1H-pyrazol-4-yl)-N-(propan-2-yl)-1,3-thiazole-4-carboxamide (Formula 299);
N-[(3R)-1-(3-methoxypropanoyl) pyrrolidin-3-yl]-5-(1-phenyl-1H-pyrazol-4-yl)-332 N-propyl-1H-pyrrole-2-carboxamide (Formula 300);
(3S)-3-methyl-1-[2-(1-methyl-1H-pyrazol-4-yl)-1,3-thiazole-4-carbonyl]piperazine (Formula 301);
(3S)-3-methyl-1-{2-[1-(propan-2-yl)-1H-pyrazol-4-yl]-1,3-thiazole-4-carbonyl}piperazine (Formula 302);
(3S)-1-[2-(1-tert-butyl-1H-pyrazol-4-yl)-1,3-thiazole-4-carbonyl]-3-methylpiperazine (Formula 303);
N-[(3R)-1-(2-methoxyethyl) pyrrolidin-3-yl]-5-(1-phenyl-1H-pyrazol-4-yl)-N-propyl-1H-pyrrole-2-carboxamide (Formula 304); and
a physiologically acceptable salt, a solvate, a hydrate, an enantiomer or a polymorph thereof.

9. A compound pharmaceutical composition comprising one or more of a physiologically acceptable salt of a compound, a solvate of the compound, a hydrate of the compound, an enantiomer of the compound, and a polymorph of the compound, wherein the compound is selected from the following:
6 N-(1-ethylpiperidin-4-yl)-N-methyl-5-(1-phenyl-1H-pyrazol-4-yl)-1H-pyrrole-2-carboxamide (Formula 1);
(3S)-3-methyl-1-[4-(5-phenyl-1,3,4-thiadiazol-2-yl)thiophene-2-carbonyl]piperazine (Formula 2);
(3S)-3-methyl-1-[4-(1-phenyl-1H-pyrazol-4-yl)-1H-pyrrole-2-carbonyl]piperazine (Formula 3);
(3S)-3-methyl-1-[5-(1-phenyl-1H-pyrazol-4-yl)thiophene-3-carbonyl]piperazine (Formula 4);
(3S)-3-methyl-1-[2-(5-phenylthiophen-2-yl)-1,3-thiazole-4-carbonyl]piperazine (Formula 7);
(3S)-3-methyl-1-[2-(2-phenyl-1,3-oxazol-5-yl)-1,3-thiazole-4-carbonyl]piperazine (Formula 13);
(3S)-3-methyl-1-(2-{1-[3-(trifluoromethoxy)phenyl]-1H-pyrazol-4-yl}-1,3-thiazole-4-carbonyl) piperazine (Formula 24);
(3S)-3-methyl-1-{2-[1-(3-methylphenyl)-1H-pyrazol-4-yl]-1,3-thiazole-4-carbonyl}piperazine (Formula 27);
(3S)-1-{2-[1-(3-chlorophenyl)-1H-pyrazol-4-yl]-1,3-thiazole-4-carbonyl}-3-methylpiperazine (Formula 30);
(3S)-3-methyl-1-{2-[1-(pyridin-2-yl)-1H-pyrazol-4-yl]-1,3-thiazole-4-carbonyl}piperazine (Formula 33);
(3S)-1-{2-[1-(4-fluorophenyl)-1H-pyrazol-4 yl]-1,3-thiazole-4-carbonyl}-3-methylpiperazine (Formula 34);
(3S)-3-methyl-1-(2-{1-[4-(trifluoromethoxy)phenyl]-1H-pyrazol-4-yl}-1,3-thiazole-4-carbonyl) piperazine (Formula 37);
(3S)-3-methyl-1-(2-{1-[3-(trifluoromethyl)phenyl]-1H-pyrazol-4-yl}-1,3-thiazole-4-carbonyl) piperazine (Formula 38);
(3S)-1-{2-[1-(4-chlorophenyl)-1H-pyrazol-4-yl]-1,3-thiazole-4-carbonyl}-3-methylpiperazine (Formula 39);
(3S)-1-{2-[1-(3-methoxyphenyl)-1H-pyrazol-4-yl]-1,3-thiazole-4 carbonyl}-3-methylpiperazine (Formula 62);
(3S)-3-methyl-1-{2-[1-(4-methylphenyl)-1H-pyrazol-4-yl]-1,3-thiazole-4-carbonyl}piperazine (Formula 63);
(3S)-3-methyl-1-[5-(1-phenyl-1H-pyrazol-4-yl)-1H-pyrrole-2-carbonyl]piperazine (Formula 67);
(3S)-3-methyl-1-(2-{1-[2-(trifluoromethyl)phenyl]-1H-pyrazol-4-yl}-1,3-thiazole-4-carbonyl) piperazine (Formula 72);
(3S)-3-methyl-1-(2-{1-[4-(trifluoromethyl)phenyl]-1H-pyrazol-4-yl}-1,3-thiazole-4-carbonyl) piperazine (Formula 75);
(3S)-1-{2-[1-(2-chlorophenyl)-1H-pyrazol-4-yl]-1,3-thiazole-4-carbonyl}-3-methylpiperazine (Formula 79);
(3S)-3-methyl-1-{2-[1-(2-methylphenyl)-1H-pyrazol-4-yl]-1,3-thiazole-4-carbonyl}piperazine (Formula 85);
(3S)-1-{2-[1-(2-fluorophenyl)-1H-pyrazol-4 yl]-1,3-thiazole-4-carbonyl}-3-methylpiperazine (Formula 96);
(3S)-3-methyl-1-[5-(1-phenyl-1H-pyrazol-4-yl) furan-2-carbonyl]piperazine (Formula 102);
N-(1-ethylpiperidin-4-yl)-4-(1-phenyl-1H-pyrazol-4-yl)thiophene-2-carboxamide (Formula 103);
N-(1-ethylpiperidin-4-yl)-5-(1-phenyl-1H-pyrazol-4-yl) furan-2-carboxamide (Formula 109);
(3S)-3-methyl-1-[4-(1-phenyl-1H-pyrazol-4-yl)thiophene-2-carbonyl]piperazine (Formula 113);
(3R)-3-methyl-1-[2-(1-phenyl-1H-pyrazol-4-yl)-1,3-thiazole-4-carbonyl]piperazine (Formula 118);
N,N-diethyl-1-[2-(1-phenyl-1H-pyrazol-4-yl)-1,3-thiazole-4-carbonyl]pyrrolidin-3-amine (Formula 134);
N-(1-methylpiperidin-4-yl)-2-(1-phenyl-1H-pyrazol-4-yl)-1,3-thiazole-4-carboxamide (Formula 138);
(3S)-3-methyl-1-[2-(1-phenyl-1H-pyrazol-4-yl)-1,3-thiazole-4-carbonyl]piperazine (Formula 157);
N-(1-ethylpiperidin-4-yl)-2-(1-phenyl-1H-pyrazol-4-yl)-1,3-thiazole-4-carboxamide (Formula 185);
1-ethyl-4-[2-(1-phenyl-1H-pyrazol-4-yl)-1,3-thiazole-4 carbonyl]-1,4-diazepane (Formula 193);
N-methyl-2-(1-phenyl-1H-pyrazol-4-yl)-N-[1-(propan-2-yl) piperidin-4-yl]-1,3-thiazole-4-carboxamide (Formula 195);
N-(1-ethylpiperidin-4-yl)-N-methyl-2-(1-phenyl-1H-pyrazol-4-yl)-1,3-thiazole-4 carboxamide (Formula 196);
N,N-dimethyl-1-[2-(1-phenyl-1H-pyrazol-4-yl)-1,3-thiazole-4-carbonyl]piperidin-4-amine (Formula 197);
N-[(3S)-1-methylpiperidin-3-yl]-2-(1-phenyl-1H-pyrazol-4-yl)-1,3-thiazole-4 carboxamide (Formula 208);
1-[2-(1-phenyl-1H-pyrazol-4-yl)-1,3-thiazole-4-carbonyl]-4-(propan-2-yl) piperazine (Formula 209).

10-11. (canceled)

12. A pharmaceutical composition according to claim 1, further comprising a pharmaceutically acceptable carrier.

13. A pharmaceutical composition according to claim 1, wherein the compound is for use in at least one prevention and treatment of diseases in a mammalian subject.

14. A pharmaceutical composition according to claim 1, wherein the compound is for use as an autophagy inducer, wherein the use is at least one of cosmetic and in vitro.

15. A pharmaceutical composition according to claim 1, wherein the compound is for use in at least one of prevention and treatment of an autophagy-related disease or condition in a mammalian subject.

16. The pharmaceutical composition according to claim 15, wherein the autophagy-related disease or condition is selected from the group consisting of neurodegenerative diseases, Huntington's disease, Alzheimer's disease, Parkinson's disease, epilepsy, brain cancer, neuronal cancer, Wilson Disease, viral infection, viral-infection-related neuronal diseases, Niemann-Pick type C (NPC) disease, autoimmune diseases, multiple sclerosis, stroke, Wegener's granulomatosis, neuropathic pain, post-operative phantom limb pain, postherpetic neuralgia, ALS, spinal cord injury, and diseases and conditions of at least one of the central nervous system and neurons involving at least one of misfolded proteins and nonfolded proteins.

17. A pharmaceutical composition for use according to claim 1, further comprising at least one additional pharmaceutically active substance for the autophagy-related disease or condition.

18. A pharmaceutical composition for use according to claim 15, wherein the at least one of prevention and treatment further comprise at least one detecting monitoring in the subject a response of at least one autophage-biomarker selected from the group of BECN1 and ATG8/LC3 family comprising LC3A, LC3B, LC3C), LC3-II, ULK1, p62, NBR1, ATG5 and ATG7.

19. A pharmaceutical composition according to claim 1, wherein the compound exhibits high concentration levels in the central nervous system and brain after administration thereof.

20-22. (canceled)
